(19) United States
(12) Patent Application Publication
(10) Pub. No.: US 2020/0146937 A1
(43) Pub. Date: May 14, 2020

(54) MEDICAL VESSELS WITH PERMANENT ENGAGEMENT MECHANISMS

(71) Applicant: Mikael NAYGAUZ, Forrest Hills, NY (US)

(72) Inventor: Mikael NAYGAUZ, Forrest Hills, NY (US)

(21) Appl. No.: 16/233,923

(22) Filed: Dec. 27, 2018

Related U.S. Application Data
(60) Provisional application No. 62/758,658, filed on Nov. 11, 2018.

(51) Int. Cl. A61J 1/20; A61J 1/14; A61L 2/26

(52) U.S. Cl. CPC A61J 1/2089; A61J 1/145; A61L 2/26; A61J 1/1481

(57) ABSTRACT

The present invention relates, in some embodiments thereof, to devices, systems and methods with a locking mechanism that allows a secure connection between vessels. In some embodiments, the locking mechanism allows a secure engagement between a vessel and a cartridge. In some embodiments, the locking mechanism allows a secure engagement between a vessel and a decontamination device. In some embodiments, the locking mechanism allows a secure engagement between two vessels.

### CROSS REFERENCE TO RELATED APPLICATIONS

This application claims the benefit of U.S. Provisional Patent Application No. 62/758,658 filed on Nov. 11, 2018 U.S. application Ser. No. 16/100,840 filed on Aug. 10, 2018; and U.S. application Ser. No. 16/100,964 filed on Aug. 10, 2018.

The contents of the above applications are all incorporated by reference as if fully set forth herein in their entirety.

### FIELD OF THE INVENTION

The present invention relates, in some embodiments thereof, to devices, systems and methods that allow a secure connection between vessels. In some embodiments, the devices, systems and methods allow a secure engagement between a vessel and a cartridge. In some embodiments, the devices, systems and methods allow a secure engagement between a vessel and a decontamination device.

### BACKGROUND OF THE INVENTION

The medicinal practice routinely involves administration of medical substances, such as, medicaments, fluids, nutrition substances and the alike, to patients. The preparation and/or administration of such medicinal substances typically involves one or more transfers of those substances between vessels (such as, vial, syringes, infusion lines, etc.). Each such act of transferring exposes connection interfaces and/or surfaces of the vessels and accordingly the medical substances themselves to contaminants present in ambient air particles (e.g., bacteria, viruses, funguses, spores, pyrogens). In addition, connection interfaces are further prone to contaminations due to physical contact of the interfaces, for example, with nonsterile gloves, or devices.

Such contaminations are a major problem in the healthcare setting since contaminants, once invading within medicinal substances, may pose substantial danger if administered intracorporeally to patients.

Typical connection interfaces of medicaments include rubber bungs/stoppers covered by a cap/seal that can be flicked off/removed prior to usage thereof. Those rubbers are used to allow penetration by a needle attached to a syringe or by other medical connectors. When the cap is flicked off/removed, the rubber bung/stopper is exposed to ambient air and to contaminants presents therein. Accordingly, exposure of connection interfaces to ambient air may involve contamination of the interfaces and consequently contamination of a beneficial substance to be provided to a patient.

Existing systems include USD720067; USD717947; USD703812; USD690418; USD639939; USD637713; U.S. Pat. Nos. 9,790,011; 9,775,777; 9,561,326; 9,493,281; 9,492,353; 9,309,020; 9,173,816; 9,168,203; 9,162,803; 9,039,672; 8,926,583; 8,827,978; 8,790,330; 8,662,985; 8,657,803; 8,622,985; 8,562,583; 8,545,475; 8,523,838; 8,491,563; 8,480,646; 8,449,521; 8,381,776; 8,336,587; 8,328,772; 8,287,513; 8,225,826; 8,075,550; 8,029,747; 7,998,134; 7,975,733; 7,942,860; 7,867,215; 7,744,581; 7,731,678; 7,387,216; 7,306,584; 6,875,203; 6,729,370; 6,715,520; 6,602,239; 6,409,708; 6,343,629; 6,162,199; 6,113,583; 6,063,068; 5,893,397; 5,876,380; 5,832,971; 5,807,374; 5,746,733; 5,569,235; 5,462,535; 5,405,326; 5,292,318; 5,279,582; 4,944,723; 4,932,947; 4,932,937; 4,919,657; 4,915,701; 4,826,489; 4,673,404; 4,564,054; 3,610,241; 3,605,743; 3,587,575; 3,583,399; 3,578,037; 3,556,099; 3,552,387; 3,406,686; 3,380,450; 3,375,825; 3,342,180; 3,330,282; 3,330,281; 3,306,290; 3,255,752; 3,253,592; 3,076,456; 2,972,991; 2,922,419; US20160262982; US20160038373; US20150209568; US20140183196; US20140016570; US20140007973; US20140000754; US20130184672; US20130006200; US20120209238; US20120209218; US20120203194; US20110284561; US20110186177; US20110125128; US20110108158; US20110098647; US20100249745; US20100198182; US20100152669; US20100147402; US20100036319; US20100004602; US20090057258; US20080312634; US20080223484; US20080171981; US20060276759; US20050215976; US20030199847; US20030187420; US20020130100; US20020115981; US20020099354; ES2577377T3; EP2852367B1; EP2666513; EP2155141B1.

In order to overcome this obstacle, the current medical practice involves swabbing the surface of a connection interface with a disinfecting agent, such as 70% isopropyl alcohol, prior to accessing the connection interface. Other methods include i.v. (intravenous) rooms which are used for the sterile preparation of i.v. medications. Such rooms, to keep medicinal preparations as sterile as possible, should be equipped with special instruments including, hoods with air filtrations (e.g., HEPA filters), ventilation systems and air pressure systems. Additionally, those rooms necessitate the medical staff to be properly garmented, use aseptic techniques, and employ quality control and validation processes. These systems are therefore expensive, labor intensive, and require regular maintenance and testing to assure that they are operating effectively. The above described systems and methods are either cumbersome and expensive or inefficient in addressing the problem of reducing/eliminating contaminants on connection interfaces.

Additionally, engagement between medicinal containers (such as intravenous bags and/or vials), engagement of connectors to medicinal containers, and/or engagement of medical devices (such as but not limited to syringes, buretrols, etc.) to medicinal containers and/or connectors may not be permanent thus allowing the detachment of the mentioned containers, connectors, and/or devices and spillage of a medicinal substance from within the containers, connectors, and/or devices into the environment and exposure of a user to the medicinal substance. The spillage of a medicinal substance is particularly dangerous if the medicinal substance is a toxic substance, a teratogenic substance, and the alike. In one example, the toxic substance may be a chemotherapeutic substance.

There is a need for reliable, user friendly and cost-effective solutions allowing a secure fluid transfer between vessels. There is further a need for such improved devices which afford fluid transfer in a contaminant free and secure manner. The herein described invention provides a solution to such long felt needs.

### SUMMARY OF THE INVENTION

It is an object of the invention, in one or more embodiments, to provide containers, devices, systems and methods which allow to transfer medical substances between vessels in a secure manner.

It is an object of the invention, in one or more embodiments, to provide, systems and methods which allow to securely transfer medical substances between vessels in a decontaminated manner.

It is an object of the invention, in one or more embodiments, to provide, devices, systems and methods which allow to securely load/reload a vessel with a medical substance.

Objects of one or more embodiments of the invention are achieved by providing containers, devices, systems and methods which include a locking mechanism and/or a securing mechanism allowing to connect vessels, in a simple, reliable and secure manner. The herein disclosed locking mechanism includes a locking member which can connect a securing member and/or alternately a securing member which can connect a locking member.

In one or more embodiments, the locking mechanism provides a secure connection between vessels. In one or more embodiments, the locking mechanism provides a permanent connection between vessels. In one or more embodiments, the locking mechanism provides an irreversible connection between vessels.

In a first aspect the present invention provides a system for the engagement of vessels, the system comprising:
a first vessel;
a second vessel configured to be coupled to the first vessel; and
a locking mechanism configured to permanently connect the vessels to each other, the locking mechanism comprises a locking member and a securing member, each disposed on a separate vessel, wherein the locking member configured to be coupled to the securing member and the securing member configured to permanently hold the locking member.

In one or more embodiments, the first vessel is a container and the second vessel is a container.

In one or more embodiments, the first vessel is a bag and the second vessel is a bottle.

In one or more embodiments, the first vessel is a container and the second vessel is a container having a plunger.

In one or more embodiments, the locking member is selected from a hook, a tooth, a pin, a knob, a ratchet tooth, a thread, a bangle, a locking key, and a zip-tie. In one or more embodiments, the locking member is disposed on a container. In one or more embodiments, the locking member is disposed within a cavity of the container. In one or more embodiments, the container is a bag or a bottle. In one or more embodiments, the container is a cartridge with a moveable wall/plunger.

In one or more embodiments, the securing member is selected from a dent, a hole, a channel, and a slot. In one or more embodiments, the securing member is disposed on a container. In one or more embodiments, the securing member is disposed within a cavity of the container. In one or more embodiments, the container is a bag or a bottle. In one or more embodiments, the container is a cartridge with a moveable wall/plunger.

In one or more embodiments, the locking member is moveable between a retracted position wherein the locking member abuts a wall of the vessel and an extended position, wherein the locking member protrudes from a wall of the vessel.

In one or more embodiments, at least one of the locking member and securing member disposed in an external position of the vessel.

In one or more embodiments, at least one of the locking member and securing member disposed in an internal position of the vessel.

In one or more embodiments, the locking member is pivotally connected to the vessel and moveable about the pivot.

In one or more embodiments, the locking member is spring loaded or attached to a spring, allowing planar movement of the locking member.

In one or more embodiments, the system further comprising an engagement mechanism to connect the first vessel to the second vessel.

In one or more embodiments, the engagement mechanism is selected from a thread, a luer, a ratchet teeth mechanism, a retention member, an adhesive mechanism, a clamping member and combinations thereof.

In one or more embodiments, the engagement mechanism disposed in an external position of the vessel.

In one or more embodiments, the engagement mechanism disposed in an internal position of the vessel.

In one or more embodiments, the vessel is selected from the group consisting of a bottle, a syringe, an infusion line, a connector, a filter, a manifold, a bag port, a bottle port, a vial port, a cartridge and a combination thereof.

In one or more embodiments, the first and/or second vessel includes a port having peripheral walls enclosing a cavity, the cavity configured to receive or engage with a vessel, or a portion thereof.

In one or more embodiments, the locking member and/or the securing member disposed on an internal and/or external position of the port.

In one or more embodiments, the first and/or second vessel includes a port having peripheral walls enclosing a cavity, the cavity configured to receive or engage with a vessel, or a portion thereof, wherein the engagement mechanism disposed on an internal and/or external position of the port.

In one or more embodiments, the first or second vessels configured to be coupled to a plurality of vessels.

In one or more embodiments, the first vessel is configured to accommodate a fluid substance and the second vessel configured to receive a fluid substance from the first vessel.

In one or more embodiments, the first vessel containing a beneficial substance and the second vessel containing a beneficial substance.

In one or more embodiments, the beneficial substance is a fluid.

In one or more embodiments, the fluid is a sterile fluid.

In one or more embodiments, the beneficial substance is a diluent.

In one or more embodiments, the beneficial substance is a medicament.

In one or more embodiments, the first vessel is a cartridge.

In one or more embodiments, the first and/or second vessel includes a port having peripheral walls enclosing a cavity, the cavity is sized and shaped to accommodate the cartridge. In one or more embodiments, when engaged, an external surface of a wall of the cartridge protrudes from an external surface of a wall of the second vessel.

In one or more embodiments, when engaged, an external surface of a wall of the cartridge is aligned with an external surface of a wall of the second vessel.

In one or more embodiments, when engaged, an external surface of a wall of the cartridge is entirely concealed within the walls of the second vessel. In one or more embodiments, a plurality of cartridges are configured to be coupled to the second vessel.

In one or more embodiments, the first vessel includes a fluid transfer mechanism for transferring a fluid substance from the first vessel to the second vessel.

In one or more embodiments, the fluid transfer mechanism includes a moveable plunger which when moved towards the first vessel's opening, allows the fluid transfer.

In one or more embodiments, the fluid transfer mechanism is a moveable wall.

In one or more embodiments, the system further comprising a safety mechanism for preventing retraction of the fluid transfer mechanism.

In one or more embodiments, a hermetically sealed connection is formed between the first vessel and the second vessel following the vessels' engagement.

In another aspect the present invention provides a system for the engagement of vessels, the system comprising:
a first vessel;
a second vessel;
a decontamination device comprising a housing configured to be coupled to the first vessel and second vessel, the decontamination device further configured to decontaminate a surface of at least one of the first and second vessels; and
a locking mechanism configured to permanently lock the decontamination device to at least one of the first vessel and second vessel, the locking mechanism comprises a locking member and a securing member, wherein one of the locking member and securing member disposed on a vessel, and the complementary locking/securing member disposed on the housing of the decontamination device, wherein the locking member configured to be coupled to the securing member and the securing member configured to permanently hold the locking member.

In one or more embodiments, the decontamination device further comprising a wiping member disposed within the housing, the wiping member configured to move within the housing.

In one or more embodiments, the decontamination device further comprising at least two compartments.

In one or more embodiments, the decontamination device further comprising a sliding mechanism configured to allow the sliding of at least one of the first vessel and the second vessel between compartments of the decontamination device.

In one or more embodiments, the sliding mechanism is a rail mechanism.

In one or more embodiments, the locking member is moveable between a retracted position wherein the locking member abuts a wall of the vessel or decontamination device and an extended position, wherein the locking member protrudes from a wall of the vessel or decontamination device.

In one or more embodiments, at least one of the locking member and securing member disposed in an external position of the vessel or decontamination device.

In one or more embodiments, at least one of the locking member and securing member disposed in an internal position of the vessel or decontamination device.

In one or more embodiments, the locking member is pivotally connected to the vessel or decontamination device and moveable about the pivot.

In one or more embodiments, the system further comprising an engagement mechanism to connect the decontamination device to the first and/or second vessel.

In one or more embodiments, the vessel and/or decontamination device includes a port having peripheral walls enclosing a cavity, the cavity configured to receive or engage with a vessel and/or decontamination device, or a portion thereof.

In one or more embodiments, the decontamination device is pre-attached to the first or second vessels.

In one or more embodiments, the decontamination device is integrally attached to the first or second vessel.

In one or more embodiments, the decontamination device includes a wiping member configured to wipe a surface of a vessel.

In one or more embodiments, the first and/or second vessel is configured to move within the housing and wherein the wiping member configured to remove contaminants from a surface of the first and/or second vessels at about the time of the vessel's movement.

In one or more embodiments, the wiping member is configured to move within the housing such to allow decontamination a surface of at least one of the first and second vessels at about the time of the wiping member's movement.

In one or more embodiments, the decontamination device includes a sliding mechanism located on and/or attached to the wiping member and/or the vessel and configured to allow for the movement of the wiping member and/or the vessel within the housing.

In one or more embodiments, when the locking member disposed on the housing it is moveable between a retracted position wherein the locking member abuts a wall of the housing, and an extended position wherein the locking member protrudes from a wall of the housing.

In yet another aspect the invention provides a method of establishing a fluid passageway between vessels, the method comprising the steps of:
providing a first vessel having a locking member;
providing a second vessel having a securing member;
engaging the first vessel to the second vessel via an engagement mechanism; and
locking the locking member of the first vessel to the securing member of the second vessel.

In one or more embodiments, an airtight seal is formed between the vessels following the step of locking and/or engaging the vessels, allowing a hermetically sealed connection between the first vessel and the second vessel.

In one or more embodiments, the first vessel is a container and the second vessel is a container.

In one or more embodiments, the first vessel is a container and the second vessel is a cartridge having a plunger.

In one or more embodiments, the first container is a bag and the second container is a bottle.

In one or more embodiments, the bottle is a vial.

In one or more embodiments the first vessel is a container and the second vessel is a syringe.

In one or more embodiments, the plunger does not have a handle.

In one or more embodiments, the step of locking the vessels includes coupling a locking member to a securing member, each disposed on a separate vessel.

In one or more embodiments, the step of engaging the first vessel to the second vessel occurs prior to locking the vessels.

In one or more embodiments, the step of engaging the first vessel to the second vessel occurs after locking the vessels.

In one or more embodiments, the step of engaging the first vessel to the second vessel occurs at about the same time as locking the locking member of the first vessel to the securing member of the second vessel.

In one or more embodiments, the step of engagement of the vessels is selected from a twisting of a thread onto a complementary thread, attaching ratchet teeth to a complementary retention member, and adhering a first connection interface to the second connection interface via an adhesive, and combinations thereof.

In one or more embodiments, the locking of the first vessel to the second vessel is via a push, a twist, a slide activation, or combinations thereof

In one or more embodiments, the method further comprising actuating a piercing member/needle to pierce the vessel surfaces.

In one or more embodiments, the method further comprising providing a decontamination device having a housing and a wiping member, the wiping member disposed within the housing and configured to decontaminate a surface of the first and/or second vessel.

In one or more embodiments, the locking and engaging of the first and second vessels is via the decontamination device, such that the decontamination device is engaged with and/or locked to the first and second vessels.

In one or more embodiments, the method further comprising actuating the wiping member to move within the housing, thereby decontaminating the surface of the first and/or second vessel.

In one or more embodiments, the method further comprising moving a surface of the first vessel and/or a surface of the second vessel within the housing and via the wiping member, thereby decontaminating the surface of the first and/or second vessel.

In one or more embodiments, the vessel is selected from the group consisting of a bottle, a syringe, an infusion line, a connector, a filter, a manifold, a bag port, a bottle port, a vial port, a cartridge and a combination thereof.

In one or more embodiments, the vessel is a container.

In one or more embodiments, at least one of the first and second vessels is a cartridge.

In one or more embodiments, the method further comprising transferring fluid between the vessels after engaging and locking the vessels.

In one or more embodiments, the method further comprising transferring fluid within the at least one vessel through the device after the surface of the at least one vessel has been decontaminated.

In yet another aspect, the invention provides a cartridge having a moveable wall without a handle.

In yet another aspect, the invention provides a cartridge having an interior made of a glass material and an exterior made of a plastic material.

In one or more embodiments, the plastic material is an elastomeric material.

In one or more embodiments, the plastic material is rigid.

In one or more embodiments, the plastic material is not rigid.

In yet another aspect, the invention provides a cartridge having an interior made of a glass material and an exterior made of a rubber material.

In yet another aspect, the invention provides a container having a thread engagement mechanism disposed along an exterior length of the container.

In one or more embodiments, the thread engagement mechanism is disposed along at least 25% of an exterior length of the container.

In one or more embodiments, the thread engagement mechanism is disposed along at least 50% of an exterior length of the container.

In one or more embodiments, the thread engagement mechanism is disposed along at least 75% of an exterior length of the container.

In one or more embodiments, the thread engagement mechanism is disposed along an entire exterior length of the container.

In one or more embodiments, the container is a vial.

In one or more embodiments, the container is a bottle.

In one or more embodiments, the container is a cartridge having a plunger.

In yet a further aspect, the invention provides a cartridge having at least one stopper disposed on an interior side wall of the cartridge, the stopper configured to ensure one-directional movement of a plunger within the cartridge.

In yet a further aspect, the invention provides a cartridge having a plunger and at least one blocker, the blocker configured to prevent detachment of the plunger from the cartridge.

In one or more embodiments, the cartridge further comprising at least one blocker, the blocker configured to prevent detachment of the plunger from the cartridge.

In yet a further aspect, the invention provides a cartridge having a plunger and at least one protective side wall.

In one or more embodiments, the protective side wall is configured to prevent a user from being exposed to a beneficial substance disposed within the cartridge.

In one or more embodiments, the cartridge has at least one plunger side wall.

In one or more embodiments, the cartridge has a circumferential plunger side wall.

In yet another aspect, the invention provides a cartridge having a plunger with a circumferential plunger side wall configured to protect a user's finger from being exposed to a substance disposed within the cartridge.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1B are front view illustrations demonstrating a system with a first vessel and a second vessel, the vessels having a locking mechanism in the form of a retractable knob and a hole, according to some embodiments of the invention;

FIGS. 2A-2B are front view illustrations demonstrating the system of FIGS. 1A-1B with an alternative locking mechanism having a hook and a dent, according to some embodiments of the invention;

FIGS. 3A-3B are front view illustrations demonstrating the system of FIGS. 2A-2B with two hooks which can be coupled into dents, according to some embodiments of the invention;

FIGS. 4A-4B are front view illustrations demonstrating the system of FIGS. 1A-1B with a locking mechanism in the form of a retractable knob and a hole, and a further locking mechanism having a retractable ball and a hole, according to some embodiments of the invention;

FIGS. 5A-5B are front view illustrations demonstrating yet another system with a locking mechanism in the form of a tooth and a dent, according to some embodiments of the invention;

FIGS. 6A-6B are front view illustrations demonstrating yet another system with an engagement mechanism with clamping rails and a locking mechanism in the form of a tooth and a dent, according to some embodiments of the invention;

FIGS. 7A-7B are front view illustrations demonstrating yet another system with an engagement mechanism in the form of a thread and a luer and a locking mechanism in the form of a tooth and a dent, according to some embodiments of the invention;

FIGS. 8A-8B are front view illustrations demonstrating yet another system with an engagement mechanism in the form of a clamping rail and a locking mechanism in the form of a knob and a dent, according to some embodiments of the invention;

FIGS. 9A-9C are front cut view illustrations demonstrating a decontamination device attached to a first vessel and configured to be coupled to a second vessel, the decontamination device and second vessel include a locking mechanism in the form of tooth-dent, according to some embodiments of the invention;

FIGS. 10A-10D are front cut view illustrations demonstrating the system of FIGS. 9A-9C with a locking mechanism in the form of tooth-dent and an engagement mechanism the form of clamping members, according to some embodiments of the invention;

FIGS. 11A-11C are front cut view illustrations demonstrating the system of FIGS. 9-10 with a locking mechanism in the form of tooth-dent and an engagement mechanism in the form of a thread and a luer, according to some embodiments of the invention;

FIGS. 12A-12C are front cut view illustrations demonstrating the system of FIGS. 9-11 with a locking mechanism in the form of tooth attached to a built-in dent structure, according to some embodiments of the invention;

FIGS. 13A-13B are front cut view illustrations demonstrating the system of FIGS. 9-12 with a locking mechanism in the form of tooth-dent and an engagement mechanism in the form of circumferential wipers, according to some embodiments of the invention;

FIG. 14 is a front view illustration demonstrating a vessel which is connected to a plurality of housings, each of which configured to connect a medical vessel, according to some embodiments of the invention;

FIG. 15 is a front view illustration demonstrating a connector which includes a vessel's locking mechanism in the form of teeth and a dent, the connector is configured to connect a first and a second vessel, according to some embodiments of the invention;

FIGS. 16A-16B are perspective bottom view illustrations demonstrating a decontamination device with three compartments, the device includes a locking mechanism in the form of bangle-channel, according to some embodiments of the invention;

FIG. 17 is a front cut view illustration demonstrating a three compartment-connector which includes a vessel's locking mechanism in the form of a teeth and a dent, the connector is configured to connect a first and a second vessel, according to some embodiments of the invention;

FIGS. 18A-18B are front cut view illustrations demonstrating a three compartment-device which includes a locking mechanism in the form of a teeth and a dent, according to some embodiments of the invention;

FIG. 19 is a side cut view illustration demonstrating a syringe's port that includes outwardly protruding teeth allowing locking with a housing of the system, according to some embodiments of the invention;

FIG. 20 is a side cut view illustration demonstrating a vial's port that includes outwardly protruding teeth allowing locking with a housing of the system, according to some embodiments of the invention;

FIGS. 21A-21B are front cut view illustrations demonstrating a decontamination device with a moveable wiping member, the device includes a locking mechanism in the form of a teeth and a dent, according to some embodiments of the invention;

FIGS. 22A-22B are side cut view illustrations of the device of FIGS. 21A-21B, according to some embodiments of the invention;

FIG. 23 is a side cut view illustration of the device of FIGS. 21A-22B which includes two teeth and two dents, according to some embodiments of the invention;

FIG. 24 is a front cut view illustration of the device of FIGS. 21A-23 which includes a tooth-dent locking mechanism, the device may attach various vessel types, according to some embodiments of the invention;

FIGS. 25A-25B is a front cut view illustration of the device of FIGS. 21A-24 which further includes a filter, according to some embodiments of the invention;

FIGS. 26A-26B is a front cut view illustration of the device of FIGS. 21A-25B which includes an alternative actuator, according to some embodiments of the invention;

FIG. 27 is a front cut view illustration of yet a further exemplary device having a wide housing with numerous ports for connecting numerous vessels, according to some embodiments of the invention;

FIGS. 28A-28D are front cut view illustrations of yet a further exemplary decontamination device having ports which are shifted from each other and lie in parallel longitudinal axes, the device includes a locking mechanism in the form of teeth-dents, according to some embodiments of the invention;

FIGS. 29A-29B are front view illustrations of yet a further exemplary decontamination device having a port with a locking mechanism in the form of teeth-dents, according to some embodiments of the invention;

FIGS. 30A-30B are front view illustrations of the device of FIGS. 29A-29B which further include a threaded engagement mechanism and a locking mechanism in the form of teeth-dents/holes, according to some embodiments of the invention;

FIGS. 31A-31D are front view illustrations of the device of FIGS. 30A-30B which further include a clamping rail mechanism, according to some embodiments of the invention;

FIGS. 32A-32B are front view illustrations of the device of FIGS. 30A-30B which further include ratchet teeth, according to some embodiments of the invention;

FIGS. 33A-33B are front view illustrations of yet a further exemplary decontamination device having a port with a locking mechanism in the form of hooks-dents, according to some embodiments of the invention;

FIGS. 34A-34B are front view illustrations of the device of FIGS. 33A-33B which has an alternative locking mechanism in the form of tooth-dent, the device further includes an engagement mechanism in the form of a thread, according to some embodiments of the invention;

FIG. 35 is a perspective view illustration of a device with a moveable wiper and a locking mechanism in the form of tooth-dent, according to some embodiments of the invention;

FIGS. 36A-36C are front cut view illustrations of a device with a locking mechanism in the form of retractable pin-dent, according to some embodiments of the invention;

FIGS. 37A-37C are front cut view illustrations of the system of FIGS. 36A-36C with a locking mechanism in the form of hook-dent, according to some embodiments of the invention;

FIGS. 38A-38B are front cut view illustrations demonstrating the system of FIGS. 36A-36C with an alternative locking mechanism having a hook and a ratchet tooth, according to some embodiments of the invention;

FIGS. 39A-39C are front cut view illustrations demonstrating yet a further exemplary system with a locking mechanism having a hook and a dent, according to some embodiments of the invention;

FIGS. 40A-40B are front view illustrations demonstrating yet another system with a locking mechanism in the form of a hook and ratchet teeth, according to some embodiments of the invention;

FIGS. 41A-41C are front view illustrations demonstrating yet another system with a locking mechanism in the form of a securing key, according to some embodiments of the invention;

FIGS. 42A-42B are front cut view illustrations demonstrating a cartridge and a vessel, the cartridge and vessel include a hooking mechanism with teeth disposed on the cartridge and dents on the vessel, when loaded into the vessel the bottom wall of the cartridge align a bottom wall of the vessel, according to some embodiments of the invention;

FIGS. 43A-43B are front cut view illustrations demonstrating the system of FIGS. 42A-42B, wherein when loaded into the vessel, a bottom wall of the cartridge is located entirely inside the vessel's cavity, according to some embodiments of the invention;

FIGS. 44A-44B are front cut view illustrations demonstrating the system of FIGS. 43A-43B, wherein when loaded into the vessel, a bottom wall of the cartridge protrudes from a bottom wall of vessel, according to some embodiments of the invention;

FIGS. 45A-45B demonstrating a cartridge and a vessel, the cartridge and vessel include a hooking mechanism with teeth disposed on the vessel and dents on the cartridge, when loaded into the vessel the bottom wall of the cartridge align a bottom wall of the vessel, according to some embodiments of the invention;

FIGS. 46A-46B are front cut view illustrations demonstrating the system of FIGS. 45A-45B, wherein when loaded into the vessel, a bottom wall of the cartridge is located entirely inside the vessel's cavity, according to some embodiments of the invention;

FIGS. 47A-47B are front cut view illustrations demonstrating the system of FIGS. 45A-45B, wherein when loaded into the vessel, a bottom wall of the cartridge protrudes from a bottom wall of vessel, according to some embodiments of the invention;

FIGS. 48A-48B are front cut view illustrations demonstrating a cartridge and a vessel, the cartridge and vessel include a hooking mechanism with teeth disposed on the cartridge and dents on the vessel, the system further includes an engagement mechanism in the form of a thread, when loaded into the vessel the bottom wall of the cartridge align a bottom wall of the vessel, according to some embodiments of the invention;

FIGS. 49A-49B are front cut view illustrations demonstrating the system of FIGS. 48A-48B, wherein when loaded into the vessel, a bottom wall of the cartridge is located entirely inside the vessel's cavity, according to some embodiments of the invention;

FIGS. 50A-50B are front cut view illustrations demonstrating the system of FIGS. 48A-48B, wherein when loaded into the vessel, a bottom wall of the cartridge protrudes from a bottom wall of vessel, according to some embodiments of the invention;

FIGS. 51A-51B are front cut views demonstrating a vessel which can be loaded with multiple cartridges, when loaded into the vessel the bottom walls of the cartridges align a side wall of the vessel, according to some embodiments of the invention;

FIGS. 52A-52B are front cut view illustrations demonstrating the system of FIGS. 51A-51B, wherein when loaded into the vessel, the bottom walls of the cartridges are located entirely inside the vessel's cavity, according to some embodiments of the invention;

FIGS. 53A-53B are front cut views demonstrating a vessel which can be loaded with multiple cartridges, the vessel and cartridge include an engagement mechanism in the form of threads, when loaded into the vessel the bottom walls of the cartridges align a side wall of the vessel, according to some embodiments of the invention;

FIGS. 54A-54B are front cut view illustrations demonstrating the system of FIGS. 53A-53B, wherein when loaded into the vessel, the bottom walls of the cartridges are located entirely inside the vessel's cavity, according to some embodiments of the invention;

FIGS. 55A-55B are front cut views demonstrating a vessel which can be loaded with multiple syringe type cartridges, when loaded into the vessel the bottom walls of the cartridges align a side wall of the vessel, according to some embodiments of the invention;

FIG. 56 is front cut view illustration demonstrating the system of FIGS. 55A-55B, wherein when loaded into the vessel, the bottom walls of the cartridges located outside the vessel's cavity, according to some embodiments of the invention;

FIGS. 57A-57C are front cut views demonstrating a vessel which can be loaded with a cartridge, the vessel-cartridge include a locking mechanism in the form of a retractable pin, according to some embodiments of the invention;

FIGS. 58A-58B are front cut views demonstrating a vessel which can be loaded with a cartridge, the vessel-cartridge include a cavity having a vial-like form for accommodating therein the vial, according to some embodiments of the invention;

FIGS. 59A-59B are front cut view illustrations demonstrating the system of FIGS. 58A-58B, which include an engagement mechanism in the form of threads disposed within the cavity and onto the vial, according to some embodiments of the invention;

FIGS. 60A-60B are front cut views demonstrating a vessel which can be loaded with a syringe type cartridge, the vessel-cartridge include a cavity having a syringe-like form for accommodating therein the syringe type cartridge, according to some embodiments of the invention;

FIGS. 61A-61B are front cut view illustrations demonstrating a vessel which can be loaded with a vial type cartridge, the cavity is made an integral part of the vessel, according to some embodiments of the invention;

FIGS. 62A-62D are front cut view illustrations demonstrating a cartridge having a fluid transfer mechanism in the form of a plunger (moveable wall), when loaded into the vessel the bottom wall of the cartridge align a bottom wall of the vessel, according to some embodiments of the invention;

FIGS. 63A-63D are front cut view illustrations of the cartridge of FIGS. 62A-62D, demonstrating, wherein when loaded into the vessel, the bottom wall of the cartridges is located entirely inside the vessel's cavity, according to some embodiments of the invention;

FIG. 64 is a front cut view illustration of a system having a vessel with cartridges loaded thereto, the cartridges have a fluid transfer mechanism in the form of a plunger and/or moveable wall, when loaded into the vessel the bottom walls of the cartridges align a side wall of the vessel, according to some embodiments of the invention;

FIG. 65 is a front cut view illustration of a system having a vessel with cartridges loaded thereto, the cartridges have a fluid transfer mechanism in the form of a plunger and/or moveable wall, when loaded into the vessel the bottom walls of the cartridges concealed within the side wall of the vessel, according to some embodiments of the invention;

FIG. 66 is a front cut view illustration of a system having a vessel with cartridges loaded thereto from an upper wall thereof, the cartridges have a fluid transfer mechanism in the form of a plunger and/or moveable wall, when loaded into the vessel the walls of the cartridges align a top wall of the vessel, according to some embodiments of the invention;

FIG. 67 is a front cut view illustration of a system having a vessel with cartridges loaded thereto from an upper wall thereof, the cartridges have a fluid transfer mechanism in the form of a plunger and/or moveable wall, when loaded into the vessel the walls of the cartridges concealed within the upper wall of the vessel, according to some embodiments of the invention;

FIGS. 68A-68C are front cut view illustrations demonstrating a cartridge having a fluid transfer mechanism in the form of a plunger supported by inner side walls, the inner side walls of the plunger are shorter in length than the outer side walls of the cartridge, according to some embodiments of the invention;

FIGS. 69A-69C are front cut view illustrations demonstrating the cartridge of FIGS. 68A-68C, the inner side walls of the plunger align the outer side walls of the cartridge when the plunger entirely pushed into the opening of the cartridge, according to some embodiments of the invention;

FIG. 70 is a front cut view illustration of a system comprising a vessel having a plurality of cavities configured to accommodate a plurality of cartridges having inner side walls, the cartridges loaded thereto from a side wall thereof, according to some embodiments of the invention;

FIG. 71 is a front cut view illustration of a cartridge with a knob and a dent, according to some embodiments of the invention;

FIG. 72 is a front cut view illustration of a cartridge with knobs and threads, according to some embodiments of the invention;

FIGS. 73A-73E are front cut view illustrations of a cartridge with stoppers, according to some embodiments of the invention;

FIGS. 74A-74C are front cut view illustrations of a cartridge with stoppers and a supported cartridge, the stoppers disposed in an inner wall of the cartridge, according to some embodiments of the invention;

FIGS. 75A-75C are front cut view illustrations of a cartridge of FIGS. 74A-74C, with stoppers and a supported cartridge having walls that align the outer walls of the cartridge following the fluid transfer, according to some embodiments of the invention;

FIGS. 76A-76C are front cut view illustrations of a cartridge with stoppers and a supported cartridge having walls that are concealed in the outer walls of the cartridge following the fluid transfer, the stoppers extend from an outer surface of the inner wall of the plunger, according to some embodiments of the invention;

FIGS. 77A-77C are front cut view illustrations of a cartridge of FIGS. 76A-76B, with stoppers and a supported cartridge having walls that align the outer walls of the cartridge following the fluid transfer, according to some embodiments of the invention.

It should be appreciated that for simplicity and clarity of illustration, elements shown in the figures have not necessarily been drawn to scale. For example, the dimensions of some of the elements are exaggerated relative to each other for clarity. Further, where considered appropriate, reference numerals have been repeated among the figures to indicate corresponding elements.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary locking mechanisms for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The present invention pertains to devices, methods and systems for connecting one or more vessels in a secure manner. Optionally, the herein disclosed devices and systems pertain to a locking and/or connection of two vessels. Optionally, the herein disclosed devices and systems pertain to a locking of and/or connection between a vessel and a decontamination device. Optionally, the herein disclosed devices and systems pertain to a locking of and/or connection between a vessel and a cartridge. In one or more embodiments, the locking and/or connection allows a fluid passageway between the vessels. In one or more embodiments, the herein disclosed device and systems include a locking mechanism that can lock together two vessels. In one or more embodiments, the locking mechanism includes a locking member and a securing member.

In one or more embodiments, the locking member is configured to attach a securing member and the securing member configured to hold the locking member. Various forms of the locking member are herein contemplated. Non-limiting examples of the locking member include a hook, a tooth, a pin, a ratchet tooth, a knob, a ball, a thread, a bangle, a locking key, a latch, and a zip-tie. The locking member is optionally selected from a structure that protrudes from a surface and grips a matching dent, hole, and/or aperture.

The locking member may be optionally moveable between a retracted position and an extended position. Such design allows to conveniently engage the vessels to each other and while doing so, retracting the locking mechanism until positioning thereof to lock into/with the securing member. Optionally, the locking member extends from the securing member. For example, in cases where the securing member is a hole or the alike, the locking member may be positioned to extend from and/or through the hole. Various forms of the securing member are herein contemplated. Non-limiting examples of the securing member include a dent, a hole, an aperture, a channel and a slot.

In one or more embodiments, the locking mechanism allows a secure (i.e., the connection is stable and does not spontaneously detach) and reversible locking (i.e., by a purposely initiated action of user). In accordance with this embodiment, the herein disclosed devices and systems may be reused, and the connection may be unlocked. In one or more embodiment, the locking mechanism may allow a permanent connection (i.e., the connection cannot be detached by a simple action and requires special instrumentation to detach). In one or more embodiments, the locking mechanism may optionally allow an irreversible connection (i.e., the resulting connection cannot be detached, unless the locking mechanism is destructed). In accordance with those embodiments, the herein disclosed devices and systems are intended for a single use.

Various types, combinations and numbers of locking members and securing members may be provided in each of the herein disclosed systems, to ensure the locking action. For example, a combination of two locking mechanisms or more may be provided in each device/system/vessel.

Various types, combinations and numbers of locking members and securing members may be provided in each of the herein disclosed systems, to ensure the locking action. For example, a combination of two locking mechanisms or more may be provided in each device/system/vessel. Additionally, any of the locking members and/or securing members herein disclosed for any of the systems, containers, and/or devices may be disposed on and/or used with any of the other herein disclosed systems, containers, and/or devices.

The locking mechanism may be provided to lock or connect between a first vessel to a second vessel. In accordance with this embodiment, the locking member may be disposed on the first vessel and the securing member on the second vessel, or vice versa. Various positions of the securing/locking member on the vessels are herein contemplated. The locking/securing member may be disposed on a vessel's body, port, and/or aperture. The locking/securing mechanism may be disposed in an external or an internal position of the vessel. Optionally, any of the locking and/or securing members/mechanisms herein disclosed may be positioned within a cavity of a vessel/container.

Optionally, the locking mechanism may be provided to lock or connect between a vessel to a vessel's cartridge. In accordance with this embodiment, the locking member may be disposed on the vessel and the securing member on the cartridge, or vice versa. Various positions of the securing/locking member on the vessel and cartridge are herein contemplated. The locking/securing member may be disposed on a vessel's/cartridge's body, port, and/or aperture. The locking mechanism may be disposed in an external or in an internal position of the vessel/cartridge (such as within a cavity of the vessel).

Optionally, the vessel-vessel or vessel-cartridge connection may include in addition to the locking mechanism an engagement mechanism configured or adapted to allow an engagement between the vessels, or vessel and a cartridge. The engagement mechanism may be reversible, allowing disconnection of the vessel-vessel or cartridge-vessel. Various types of engagement mechanisms may be applicable and are contemplated. For example, the engagement mechanism is selected from, without limitation, a thread, a luer, a luer-lock, a luer-slip, snap-on, twist-on, a ratchet teeth mechanism, a retention member, a clamping rail, and an adhesive mechanism. The thread and the complementary thread may be selected from, without limitation, a luer, a smart-site mechanism and combinations thereof. In an embodiment, the engagement mechanism may be selected from a ratchet teeth mechanism, a clamping rail mechanism and/or an adhesive.

In one or more embodiments, the vessel-vessel connection may include a thread engagement mechanism having a locking mechanism. The locking mechanism may include a hook, a tooth, a pin, a ratchet tooth, a knob, a ball, a thread, a bangle, a locking key, a latch, and a zip-tie. In one or more embodiments, the vessel-vessel connection may include a thread engagement mechanism having a locking mechanism, the locking mechanism may comprise a dent, a hole, and/or an aperture. In one or more embodiments, the vessel-cartridge connection may include a thread engagement mechanism having a locking mechanism. The locking mechanism may include a hook, a tooth, a pin, a ratchet tooth, a knob, a ball, a thread, a bangle, a locking key, a latch, and a zip-tie. In one or more embodiments, the vessel-cartridge connection may include a thread engagement mechanism having a locking mechanism, the locking mechanism may comprise a dent, a hole, and/or an aperture. In one or more embodiments, the vessel-vessel connection may include a ratchet teeth and/or clamping rail engagement mechanism having a locking mechanism. The locking mechanism may include a hook, a tooth, a pin, a ratchet tooth, a knob, a ball, a thread, a bangle, a locking key, a latch, and a zip-tie. In one or more embodiments, the vessel-vessel connection may include a ratchet teeth and/or clamping rail engagement mechanism having a locking mechanism, the locking mechanism may comprise a dent, a hole, and/or an aperture. In one or more embodiments, the vessel-cartridge connection may include a ratchet teeth and/or clamping rail engagement mechanism having a locking mechanism. The locking mechanism may include a hook, a tooth, a pin, a ratchet tooth, a knob, a ball, a thread, a bangle, a locking key, a latch, and a zip-tie. In one or more embodiments, the vessel-cartridge connection may include a ratchet teeth and/or clamping rail engagement mechanism having a locking mechanism, the locking mechanism may comprise a dent, a hole, and/or an aperture.

In one embodiment, the present invention further provides devices, systems and methods configured to decontaminate one or more surfaces of a vessel(s). In one embodiment, the invention provides devices and systems configured to allow a substantially decontaminated locking of/engagement between vessels. In one or more embodiments, the devices and systems of the invention allow a substantially decontaminated fluid transfer between the one or more vessel(s). The engagement and/or locking and fluid transfer between the one or more vessels may accordingly be utilized via the decontamination devices which decontaminate the vessel(s) and also allow the fluid transfer. A locking mechanism may be disposed on the decontamination device, and one or both engaging vessels, thereby locking at least one vessel to the decontamination device. In one or more embodiments, any of the herein disclosed locking members may be disposed on and/or within a decontamination device. In one or more embodiments, any of the herein disclosed securing members may be disposed on and/or within a decontamination device.

Optionally, the locking mechanism may be provided to connect/lock between a vessel to a decontamination device. In accordance with this embodiment, the locking member may be disposed on the vessel and the securing member on the decontamination device, or vice versa. Various positions of the securing/locking member on the vessel and decontamination device are herein contemplated. The locking/securing member may be disposed, without limitation, on a vessel's/device's body, port, aperture, and/or compartment. The locking mechanism may be disposed in an external or internal position of the vessel/device. Optionally, the locking member/mechanism is disposed adjacent and/or onto a position of the engagement mechanism (such as but not limited to a thread, luer, smartsite, ratchet teeth, and/or clamping rail). Optionally, the securing member/mechanism is disposed adjacent and/or onto a position of the engagement mechanism (such as but not limited to a thread, luer, smartsite, ratchet teeth, and/or clamping rail). Optionally, the decontamination device includes in first compartment thereof a connecting structure. The connecting structure mediates a connection with a vessel, via a locking mechanism and/or via an engagement mechanism. Various forms of connecting structures are herein contemplated. In one or more embodiments, the connecting structure extends downwardly from a top inner wall of a first compartment. In one or more embodiments, the connecting structure is selected from extension bodies, clamping members, a threaded structure, minimized built-in dent structures, elongated extensions, and a circular body.

The decontamination device of the invention includes a housing having a side wall, a top wall and a bottom wall, and a wiping member disposed within the housing. The wiping member may be moveable within the housing. The decontamination device may further comprise a rail mechanism allowing movement of a vessel between compartments of the decontamination device. The device configured to be coupled to two vessels and includes applicable ports or openings for receiving the vessels. The ports or openings may optionally be formed by vertical walls extending from the top and/or bottom walls, forming an inner lumen via which medical substances may be transferred following engagement between, and decontamination of the vessel surfaces. Other port configurations include ports/openings without any non-vertical walls. The ports may be flush, or surface mounted to the housing. Optionally, a locking and/or securing member may be disposed in a port of the device, for example, in an internal position within vertical walls of the port. Optionally, a locking member and/or securing member may be disposed on an exterior surface of a port of the decontamination device. In one or more embodiments, a locking member and/or securing member and/or securing member may be disposed on any portion of the housing of the decontamination device.

The wiping member is configured to decontaminate a vessel's surface, optionally by a movement of the wiping member across the housing and surface of the vessel, or by movement of the surface of the vessel within the housing. The wiping member may be moveable or may be static.

In an embodiment of the invention, the wiping member is operable to move across and wipe off any contaminates present on the one or more surfaces of the vessels via an actuator extending through an opening of the housing. The actuator is configured to manipulate movement of the wiping member across the housing. In one or more embodiments, the wiping member is operable to move across and wipe off any contaminants present on the one or more surfaces of the vessels via an actuator extending through an opening in the side wall of the housing. Exemplary actuators include, without limitation, a handle, a tab, a button, a touch button, a lever, a gear, a spring, a mechanical actuator, an electric actuator, an air pressure actuator, and combinations thereof

In one or more embodiments, the wiping member is operable to move across and wipe off any contaminates present on the one or more surfaces of the vessels without any actuator for mediating the wiping action. In accordance with this embodiment, a user may, for example, apply force onto the housing to thereby forcibly manipulate the wiping member to slide across the housing. In accordance with this embodiment, the housing may be flexible, so the user can manipulate the wiping member from the outside and by applying for example press forces onto the housing. In one or more embodiments, the wiping member is moveable from an initial position which precedes the wiping action to an end position which follows the wiping action of the wiping member.

Optionally, the wiping member is static within the housing and the decontamination of a vessel's surface is effective via movement of the vessel within the housing and via the wiping member. In accordance with this embodiment, the system comprises a sliding mechanism positioned on at least one of the first vessel and the second vessel, and/or on and/or within the housing, the sliding mechanism configured to allow traveling there along of at least one of the first vessel and the second vessel, such that at least one of the first vessel and the second vessel is configured to move from a first position to at least a second position. The wiping member is configured to remove contaminants from a surface of at least one of the first vessel and the second vessel at about the time of movement of the medical vessels from the first position to the second position. In one or more embodiments, the medical vessel or medical vessel surface slides along the housing and passes through the wiping member under great friction, or under a friction sufficient to wipe off any contaminants from the surface of the vessel, such as dirt, bacteria or ambient air particles.

Various types of sliding mechanisms are contemplated as long as they can be connected or engage a vessel and afford traveling there along and allow decontamination of a surface of the vessel. The sliding mechanism may include a rail. The rail may be optionally made from a plastic or polymetric material, but a metallic material or glass material is also applicable. In one embodiment, the rail is manufactured from a plastic material. In one or more embodiments, the plastic material is rigid. In one or more embodiments the vessel surface is positioned on a surface of the rail. In one or more embodiments, the vessel surface is positioned between the rails of the rail. In one or more embodiments, the vessel surface is positioned between a portion of the rails. In one or more embodiments, the rail may be a circumferential rail. In one or more embodiments, the sliding mechanism may include a hinge mechanism. In one or more embodiments, the sliding mechanism provides for the travelling a vessel between a plurality of positions, such as from a second position to a third position, from a third position to a fourth position, and from a fourth position to a fifth position. In one or more embodiments, the sliding mechanism allows only unidirectional sliding. Alternatively, the sliding mechanism allows for the vessel to retract/return to a previous position.

In one or more embodiments, the wiping member is manufactured from or comprises a material including, but not limited to an elastic material, a rigid material, a flexible material, an expandable material, a polymer, and combinations thereof. In one or more embodiments, the wiping member is manufactured from or comprises an elastomeric material, such as, but not limited to a rubber material. In one or more embodiments, the wiping member is manufactured from or comprises a sponge or a sponge-like material. In one or more embodiments, the wiping member slides across the surface of the one or more vessel(s) under great friction, or under a friction sufficient to wipe off any contaminants from the surface, such as dirt or bacteria. In one or more embodiments, the vessel's surface slides underneath the wiping member under great friction, or under a friction sufficient to wipe off any contaminants from the surface, such as dirt or bacteria.

In one or more embodiments, the wiping member is covered, at least partially, by a sterilizing substance. As used herein the term “sterilizing substance” encompasses any substance that can eliminate or reduce the presence of microorganisms, air particles, or any of the alike. In one embodiment, the term “sterilizing substance” is interchangeable with the term “disinfecting substance”. In one or more embodiments, the sterilizing substance may be, but is not limited to, an antibacterial substance, an antiviral substance (e.g., an anti-HIV agent), an anti-tuberculin substance, an anti-fungal, and combinations thereof.

In one or more embodiments, the sterilizing substance may be a bactericidal substance, a virucidal substance, and/or a fungicidal substance. Exemplary sterilizing substances include, without limitation, alcohol, isopropyl alcohol, and hydrogen peroxide. In one or more embodiments, the sterilizing or disinfecting substance may be a fluid or a liquid. In one or more embodiments, the sterilizing or disinfecting substance may be a gas. In one or more embodiments, the gas may be a pressurized gas. In one or more embodiments, the sterilizing substance may be housed within the housing of the decontamination device. In one or more embodiments, at least one of the openings and/or ports of the decontamination device are sealed and/or covered thus preventing the sterilizing substance from escaping from inside the housing of the decontamination device. The seals and/or covers maintain the sterility of the decontamination device until about the time of use of the decontamination device. In one or more embodiments, the sterilizing substance is sodium hypochlorite. In one or more embodiments, the concentration of sodium hypochlorite is 0.55% or higher. In one or more embodiments, the concentration of sodium hypochlorite is below 0.55% concentration.

In one or more embodiments, the housing may be manufactured from various materials. Optionally, the housing is manufactured from an inert material, thus suitable for various types of pharmaceutical substances. The housing may be made from flexible or from rigid materials. Suitable materials include, without limitation a plastic, a glass, a rigid plastic, a flexible plastic, and combinations thereof. In one or more embodiments, the housing may be transparent. In one or more embodiments, the housing may be opaque.

Various sizes and shapes of the housing are applicable. For example, the housing may have an external most length, width, and/or height of less than about 3 inches. In one or more embodiments, the housing may have an external most length, width, and/or height of less than about 25 millimeters (mm). For example, less than about 20 mm, less than about 15 mm, or less than about 10 mm. The housing internal most length, width, and/or height may be less than about 10 mm. For example, less than about 4 mm, less than about 3 mm, or less than about 2 mm. The housing may include one or more compartments which may optionally be separated or divided by the wiping member or by any other means. In one or more embodiments, the housing includes two compartments, wherein a first compartment is kept sterile and a second compartment is configured to receive a vessel which may be contaminated. In one or more embodiments, the sterile compartment can receive a vessel following decontamination of the surface of the vessel. In one or more embodiments, the housing includes two compartments wherein a first includes a capping mechanism and the second is configured to receive a vessel. In one or more embodiments, the housing includes three, four, or five compartments. The vessel may move from a first position in which a vessel surface is located within the first compartment to a second position in which the vessel surface is located in the second compartment. Optionally, the housing includes a third compartment and the vessel surface is moveable from the second position to a third position. Optionally, the housing includes a fourth compartment, or a fifth compartment and the vessel surface is moveable from a third position to a fourth position and/or a fifth position.

In one or more embodiments, the housing is conveniently light presenting a weight of no more than about 100 grams (gr). For example, less than about 75 gr, less than about 50 mm gr, less than about 25 gr, less than about 10 gr, or less than about 5 gr. In one or more embodiments, the housing may be transparent allowing visualization of its interior, or opaque.

In one or more embodiments, the housing may be a closed chamber which forms a tight, optionally, airtight connection between the vessels. In one or more embodiments, when the ports or openings are connected to vessels or when the ports are covered by a cover or a seal, the housing may form an interior which is hermetically sealed/isolated from surrounding/environmental air. In one or more embodiments, the cover or the seal is frangible, breakable or rupturable. In one or more embodiments, the cover or the seal is pierceable by a piercing member such as a needle.

In one or more embodiments, the housing may optionally include one or more apertures, optionally covered by a filter, to thereby allow releasing air from the housing when actuating the wiping member. Optionally, the one or more apertures may be covered by a valve, to thereby allow releasing of air or pressure from within the housing when actuating the wiping member. In one or more embodiments, the valve may be a one-way valve, thereby allowing the release of air or pressure from within the housing when actuating the wiping member while preventing ambient or environmental air from entering the housing. In one or more embodiments, the filter or valve may be located in or on a wall of the housing's first, second or third compartment.

In one or more embodiments, the system or devices of the invention is provided when the housing is already connected to one or more medical vessels. In accordance with this embodiment, the one or more medical vessels may be integrally manufactured or assembled with the housing and provided or distributed to consumers as such. In accordance with this embodiment, the system may be configured to allow a connection/locking/engagement to additional one or more vessels.

In one or more embodiments, the system includes two or more housings each containing a wiping member. Optionally, the housings are disposed one above the other, providing a top housing and a bottom housing. In accordance with this embodiment, a wiping member disposed on the top housing is configured to wipe a surface of a vessel connected to the top housing and a wiping member disposed on the bottom housing is configured to wipe a surface of a vessel connected to the bottom housing.

In one or more embodiments, the system includes two or more ports which are disposed one above the other, or which are longitudinally aligned with respect to each other. In accordance with this embodiment, the vessels connected to such ports are positioned aligned allowing a direct and aligned fluid passageway between the vessels. In one or more embodiments, the system includes two or more ports which are shifted from each other. Yet, in accordance with this embodiment, a fluid passageway is formed between vessels which is twisted or curved. In yet one or more embodiments, the housing contains a plurality of ports configured for connecting a plurality of vessels to the housing. The system or device is configured so that actuating the wiping member allows wiping the surfaces of all vessels connected to the housing in one action.

In one or more embodiments, the housing has an engagement mechanism configured or adapted to connect the at least one vessel to the housing. In one or more embodiments, the engagement mechanism may be reversible, allowing to detach the engagement between a vessel and a decontamination device. Various types of engagement mechanisms may be applicable and are contemplated. For example, the engagement mechanism is selected from, without limitation, a thread, a luer, a luer-lock, a luer-slip, snap-on, twist-on, a ratchet teeth mechanism, a retention member, a clamping rail, and an adhesive mechanism. The thread and the complementary thread may be selected from, without limitation, a luer, a smart-site mechanism and combinations thereof. In an embodiment, the engagement mechanism may be selected from a ratchet teeth mechanism, a clamping rail mechanism and/or an adhesive. In one or more embodiments, the herein disclosed invention allows transferring medical substances in a contaminant-free, or in a substantially contaminant-free manner.

In one or more embodiments, the herein disclosed invention affords engagement of vessels in a contaminant-free, or in a substantially contaminant-free manner. In one or more embodiments, the herein disclosed invention affords an engagement of vessels in an air-free, or in a substantially air-free manner.

In one or more embodiments, the herein disclosed invention affords locking of vessels in a contaminant-free, or in a substantially contaminant-free manner. In one or more embodiments, the herein disclosed invention affords locking of vessels in an air-free, or in a substantially air-free manner. In one or more embodiments, the herein disclosed invention affords permanent or irreversible locking of vessels in a contaminant-free, or in a substantially contaminant-free manner. In one or more embodiments, the herein disclosed invention affords permanent or irreversible locking of vessels in an air-free, or in a substantially air-free manner.

In one or more embodiments, the herein disclosed invention provides a fluidic passageway or communication between vessels in a contaminant-free, or in a substantially contaminant-free manner. In one or more embodiments, the herein disclosed invention provides a fluidic passageway or communication between vessels in an air-free, or in a substantially air-free manner.

In one or more embodiments, the herein disclosed invention, allows to substantially decrease the chances to introduce contaminants within a medical substance, when preparing or administering medical substances for administration to patients.

As used herein the term “substances” refers to various types of materials that should be kept sterile. The substances may be liquid, semi-solid, or gas. In one or more embodiments, the substances are “medical substances”. As used herein the term “medical substances” refers to and encompasses any of the various pharmaceutical drugs, fluids, nutritional products and the like. In an embodiment of the invention the term “medical substance” is interchangeable with the term “pharmaceutical substance”

As used herein the term “contaminant-free” is interchangeable with the term “sterile”, “disinfected”, and “decontaminated”. The term refers to substances that are free or substantially free of ambient air particles and/or pathogens and/or micro-organisms, and/or any of the alike. Typically, when less or no air is introduced within medical substances, the chances of contamination by pathogens, such as, bacteria, viruses, funguses, spores, pyrogens or the alike is completely abolished or significantly reduced.

As used herein, the term “substantially contaminant-free” means significantly less ambient air present when transferring medical substances with the herein disclosed vessels and systems, as compared to comparable conditions for transferring medical substances without the herein disclosed vessels and systems.

As used herein the term “ambient air particles” is interchangeable with the term “environmental air particles” and refers to air particles present in a non-filtered environment. The air particles may contain dirt, such as dust. Alternatively, or additionally, the air particles may contain pathogens or other microorganisms, or any of the alike.

As used herein the term “vessel” refers to any device utilized for containing, transferring or administering substances as herein disclosed. In one or more embodiments, the vessels may be used for containing medical substances. In an embodiment of the invention, the vessel is a medical vessel. In one or more embodiments, the term “medical vessel” is interchangeable with the term “pharmaceutical vessel”. In an embodiment of the invention, the vessel is a medical device. In an embodiment of the invention, the vessels are used for, and adapted to allow connection to another vessel. In an embodiment of the invention, the vessel is utilized for containing or housing a medical substance. Various types of vessels are contemplated. The vessel may be selected, without limitation, from a vial, a bag, a chamber, a bottle, a cartridge and the alike. In an embodiment of the invention, the term vessel further encompasses elements that can be used to connect between vessels. In accordance with this embodiment, the vessel may be selected, without limitation, from a connector, a connector having a plurality of openings, a buretrol, a syringe, an infusion line, a tubing, an infusion line, a spike, a syringe, a filter, a port and a manifold.

As used herein the term “fluid communication” refers to two or more vessels in which substances may pass therethrough either directly or indirectly. The fluid communication may occur via a fluid passageway that allows for the flow/transfer of substances. In an embodiment of the invention, fluid passageway between vessels is established when any seals or covers of the ports of the herein disclosed systems and/or of the vessels are open. Optionally, the vessels and/or ports of the systems are provided with seals/covers and those seals/cover become open upon piercing by a piercing member or breaking/rupturing thereof. Optionally, the vessels and/or ports of the systems are provided with seals/covers and those seals/ covers are opened upon uncovering or removing thereof by a user. Optionally, one or more of the seals/covers disclosed herein are fragile, allowing piercing thereof with a sharp instrument (i.e., a piercing member) or by the application of a sufficient force to break or rupture the seals/covers. The piercing member may optionally be a needle, for example, a hollowed needle, but other sharp elements are contemplated. In one or more embodiments, the seals/covers may be peelable.

In an embodiment of the invention, the herein disclosed devices, systems and methods allow fluid communication in a contaminant-free, or in a substantially contaminant-free manner. For example, between three or more, four or more, five or more or six or more vessels. In an embodiment of the invention, the herein disclosed devices, systems and methods allow fluid communication between three, four, five six, or seven vessels. For example, the devices, systems, and methods of the invention allow fluid communication between two bags, between two containers, between two bottles, between a syringe and a bag, between a syringe and a container, between a syringe and a vial, between a connector and a bag, between a container and a bag, between a vial and a bag, between a container and an infusion line or between a syringe and a connector. In an embodiment of the invention, the herein disclosed devices and systems are disposable. In an embodiment of the invention, the herein disclosed devices and systems are non-disposable.

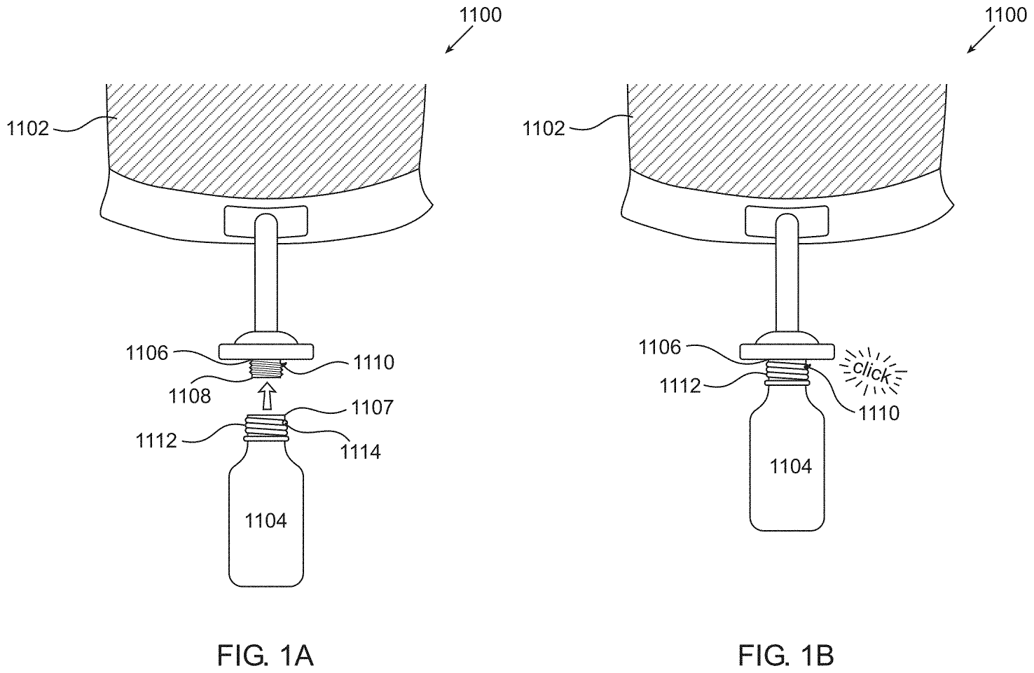

Referring now to the drawings, FIGS. 1A-8B and FIGS. 36A-41C illustrate various forms of permanent or irreversible locking mechanisms between vessels, wherein a locking member fits a securing member such that when engaged with each other a permanent or irreversible connection is formed. As will be presented herein below, various forms of locking member and securing members are herein contemplated and may be applicable.

FIGS. 1A-1B illustrate a system 1100 for allowing an irreversible or permanent engagement between vessels. First vessel 1102, being an infusion bag and second vessel 1104 being a vial are shown, but any type of pharmaceutical/medical vessel could be used and is herein contemplated, such as a bottle, an infusion line, a connector, a filter, a manifold, etc. Bag 1102 and vial 1104 each include a port 1106 and 1107, respectively, with peripheral vertical walls surrounding an opening. Bag 1102 and vessel 1104 are configured to engage with each other via an engagement mechanism disposed on the ports 1106 and 1107 and having a male thread 1108 at port 1106, and a complementary female luer 1112 at port 1107 of vial 1104. Such an engagement mechanism allows stable connection between the vessels but is reversible. A permanent connection of system 1100 includes a locking member in the form of knob 1110 sized and shaped to allow insertion thereof within a securing member in the form of hole 1114. Knob 1110 is disposed on the external surface of thread 1108, such to outwardly protrude from thread 1108. Knob 1110 may be disposed on at least one threads 1108 or between threads 1108. Knob 1110 may be made of a rigid material, optionally a semi-rigid material. The material may be a plastic, a metal, and combinations thereof. Hole 1114 disposed in port 1107 and is configured for accommodating therein or inserting therethrough knob 1110 following engagement between vessel 1102 and vessel 1104. Hole 1114 may be disposed on thread/luer 1112 or between threads/luers 1112. The resulting engagement is permanent such that the vessels cannot be disconnected spontaneously. FIG. 1A illustrates vessels 1102 and 1104 when spaced apart. FIG. 1B illustrates the vessels 1102 and 1104 when engaged with each other. The vessels are firstly screwed with each other and then locked to each other in a permanent manner via knob 1110 being inserted within hole 1114. The knob 1110 can be retracted or folded against thread 1108 to allow the vessels coupling by threading. The locking action can be obtained upon positioning hole 1114 to overlap knob 1110 and insertion and extension of knob 1110 within/through hole 1114. As shown in FIG. 1B, an audible sound such as a “click” sound or any other sound may be heard at about the time, or at the time of engagement or locking the vessels 1102 and 1104. Following the engagement between, and locking of vessels 1104 and 1102, a fluid passageway may be formed, allowing exchange of medical substances in a safe and secure manner.

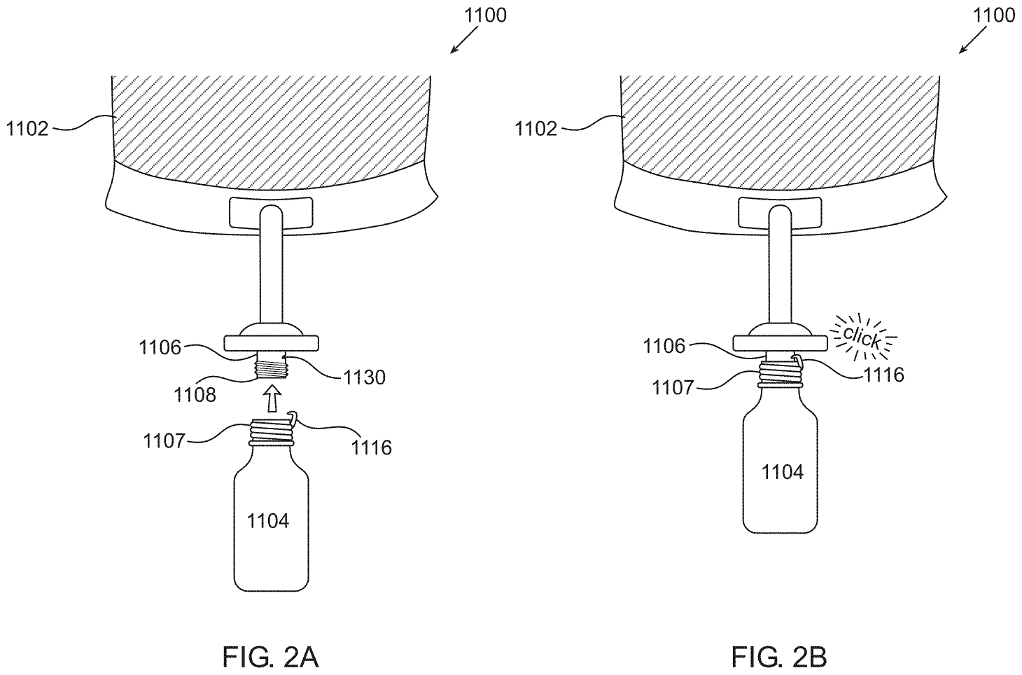

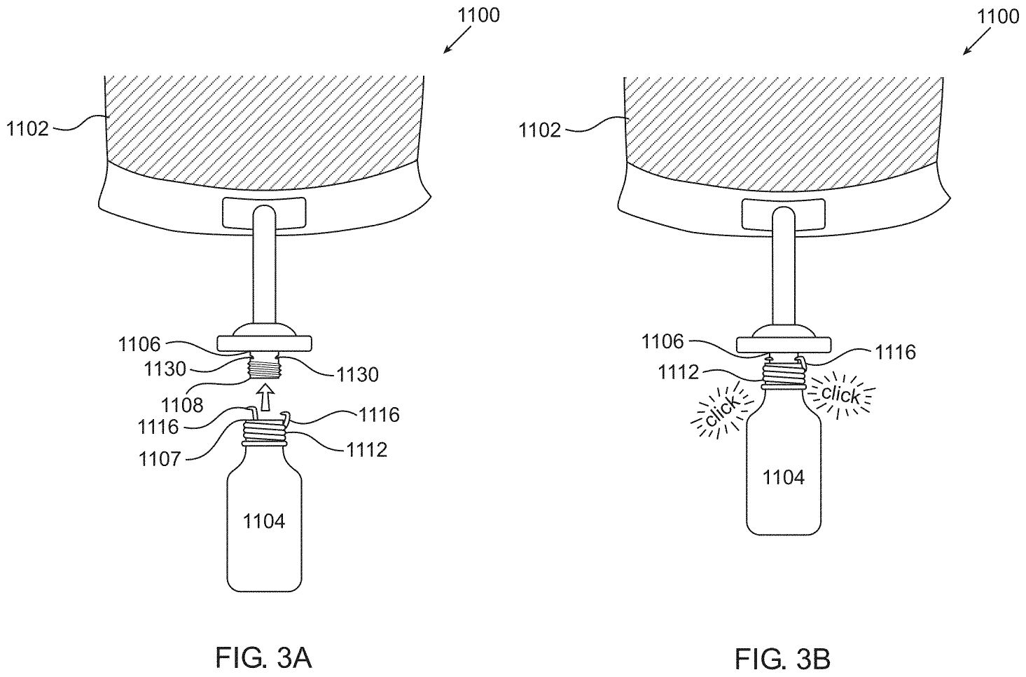

FIGS. 2A-2B, illustrate an alternative locking mechanism of system 1100 having a hook 1116, being a locking member and dent 1130, being a securing member. The hook 1116 can attach to dent 1130 in a permanent manner following engagement between vessels 1102 and 1104. As shown in FIG. 2B, an audible sound such as a “click” sound or any other sound may be heard at about the time, or at the time of engagement or locking of the vessels 1102 and 1104. One or more hooks 1116 may be disposed on port 1107. For example, as shown in FIGS. 3A-3B, two hooks 1116 may be disposed on the circumference of female luer 1112, which may hook into dents 1130. Optionally, three or more hooks, or four or more hooks are applicable and can be provided on port 1107. As shown in FIG. 3B, an audible sound such as a “click” sound or any other sound may be heard at about the time, or at the time of engagement/locking of the vessels 1102 and 1104. Hooks 1116 may be positioned on an exterior surface on exterior surface of female luer 1112 and/or on interior surface of port 1107.

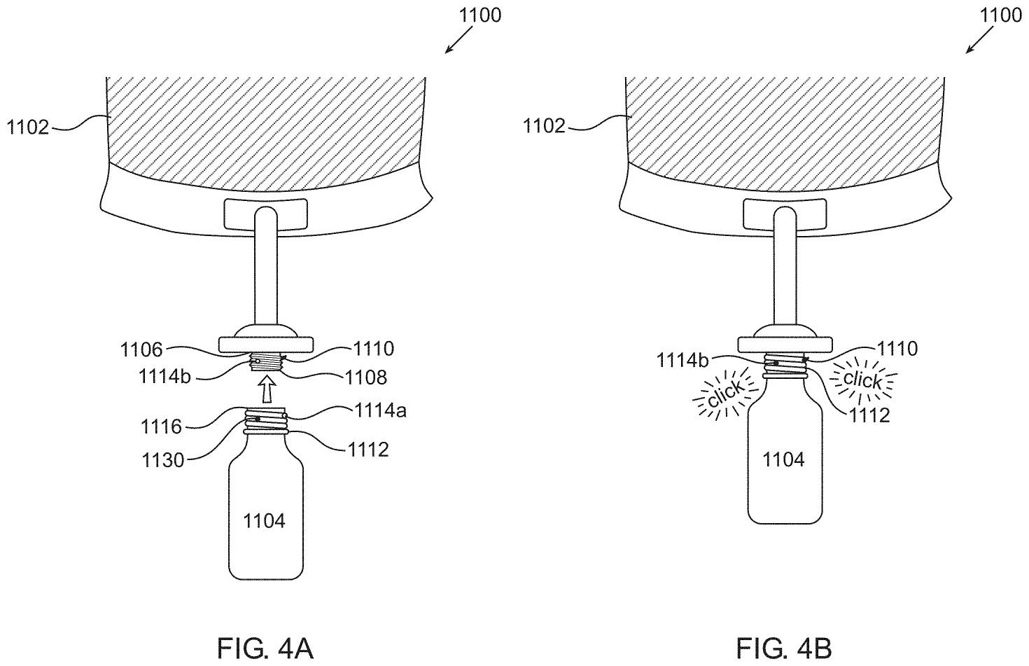

FIGS. 4A-4B, illustrate a combination of two different locking mechanisms to ensure a secure locking of the vessels. A first locking mechanism in the form of knob 1110 which is inserted within first hole 1114a and a second locking mechanism in the form of retractable ball 1130 which can be inserted within second hole 1114b are shown. Similarly, to knob 1110, ball 1130 may be moveable between a retracted position and an extended position. When in a retracted position, the ball 1130 flattens against the surface of female luer 1112, allowing the vessels 1102 and 1104 to engage with each other, via the thread-luer mechanism. When in an extended position, the ball 1130 protrudes from the outer surface of female luer 1112 and/or is inserted through hole 1114b. The knob 1110-hole 1114a and retractable ball 1130-hole 1114b provide for an irreversible connection between the vessels 1102 and 1104. Optionally, an audible sound, such as a “click” sound or any other sound may be heard at about the time or at the time of engagement/locking of the vessels 1102 and 1104 (shown in FIG. 4B).

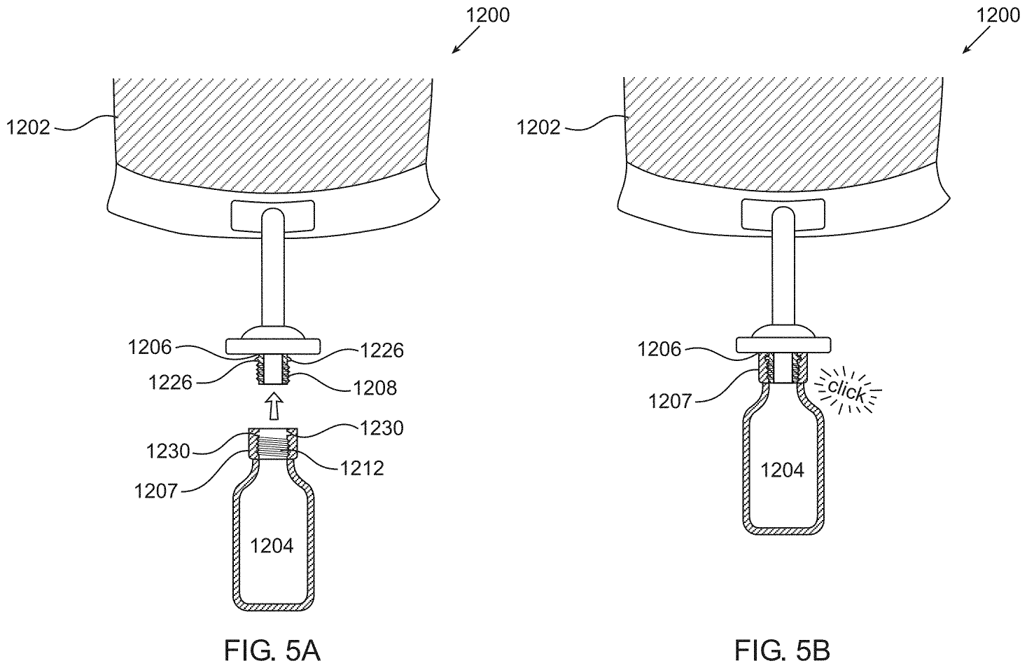

In FIGS. 5A-5B yet another form of locking mechanism having a tooth-dent configuration is shown in system 1200. The engagement mechanism with a male external thread 1208 on port 1206 is configured to cooperate with a female luer 1212 on port 1207. The locking mechanism in the form of protruding tooth 1226 positioned after male thread 1208 fits a complementary dent 1230 extending from an end of port 1207, just above female luer 1212. The tooth 1226 coupled to dent 1230, provides an irreversible engagement between the vessels 1202 and 1204. Optionally, an audible sound, such as a “click” sound or any other sound may be heard at about the time or at the time of engagement/locking of the vessels 1202 and 1204 (shown in FIG. 5B). Tooth 1226 may be made of a rigid material, optionally a semi-rigid material. The material may be a plastic, a metal, and combinations thereof. Dent 1230 may be disposed on an interior wall of port 1207 so as to prevent manual manipulation and/or detachment of the tooth-dent coupling/connection by a user.

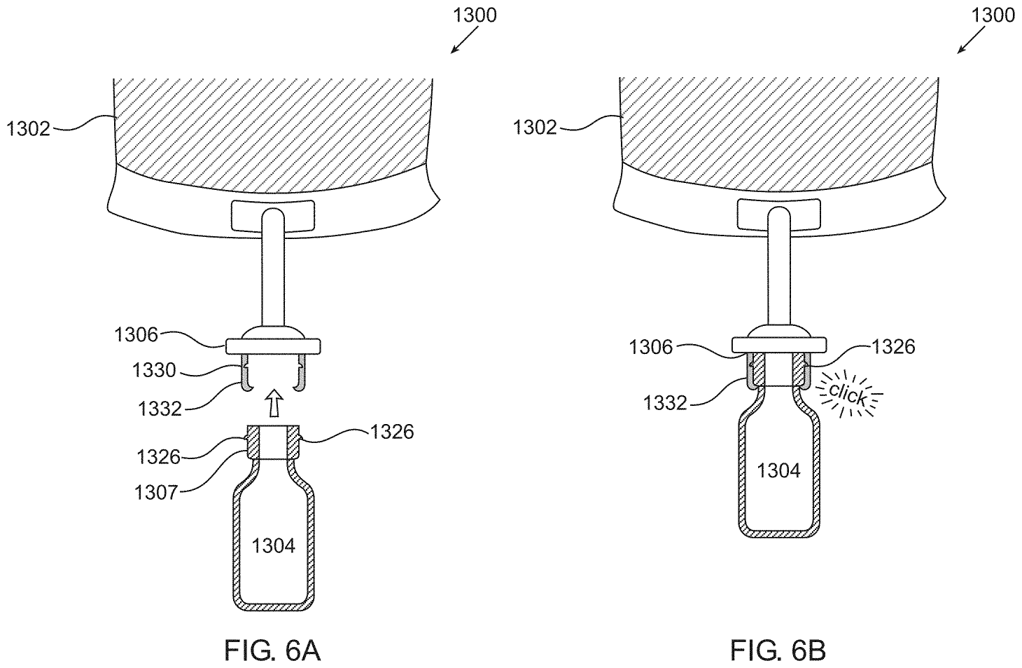

FIGS. 6A-6B illustrate system 1300 with a port 1306 (without a thread) that has clamping member 1332 which is configured to allow an engagement between vessels 1302 and 1304. The clamping member 1332 can clamp port 1307 of vessel 1304. A tooth 1326 disposed to outwardly protrude from the periphery of port 1307 is configured to cling on dent 1330 that is provided internally within clamping member 1332. The resulting engagement between vessel 1302 and vessel 1304 is permanent and stable. Tooth 1326 may be made of a rigid material such as a metal, a metal alloy, a plastic, and combinations thereof. Optionally, an audible sound, such as a “click” sound or any other sound may be heard at about the time or at the time of engagement/locking of the vessels 1302 and 1304 (shown in FIG. 6B).

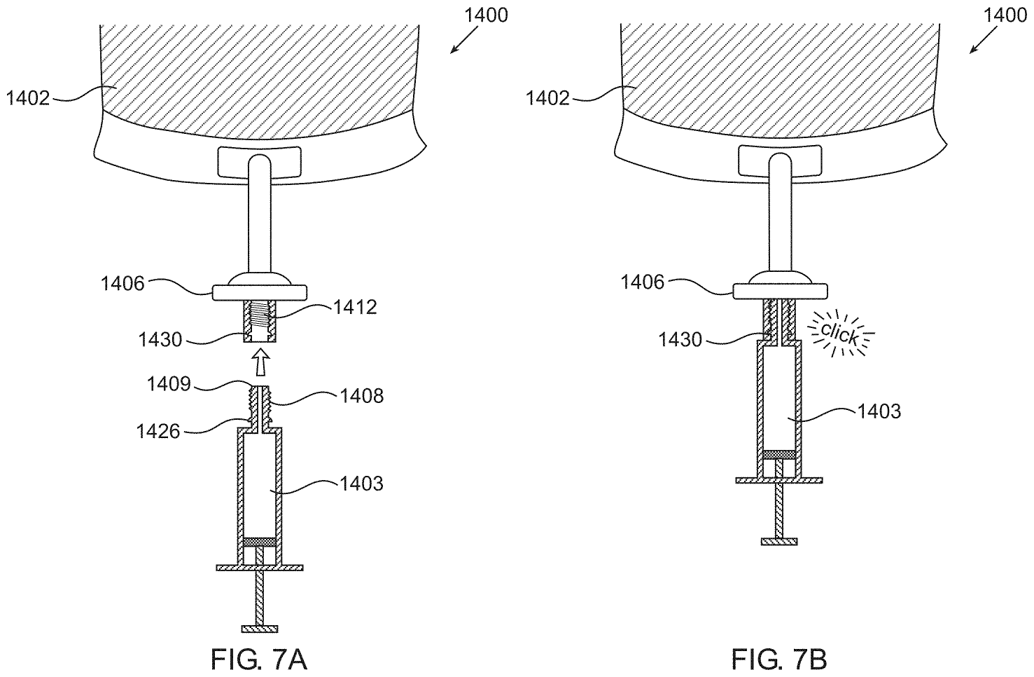

In FIGS. 7A-7B a system 1400 with a locking mechanism with tooth 1426 and dent 1430 is shown. Dent 1430 is disposed on an interior surface of port 1406 of vessel 1402. Optionally, port 1406 having dent 1430 may abut a wall of vessel 1402. Optionally, port 1406 having dent 1430 may be flush mounted or surface mounted to vessel 1402. Thread 1408 extends from an end of port 1409 of syringe 1403 and tooth 1426 is disposed just after the thread 1408. Port 1406 includes a complementary thread having a female luer 1412 and dent 1430 disposed from an end of the port 1406. Similar to the mechanism shown in FIGS. 5A-5B, the tooth 1426 attached to dent 1430, provide an irreversible engagement between the vessels 1402 and 1403. Optionally, an audible sound, such as a “click” sound or any other sound may be heard at about the time or at the time of engagement/locking of the vessels 1402 and 1404 (shown in FIG. 7B). Vessel 1402 is shown as a bag but may be any vessel such as a bottle, vial, container, buretrol, manifold, filter, connector, etc. Vessel 1403 is shown as a syringe but may be any vessel such as a bottle, vial, bag, container, buretrol, manifold, filter, connector, etc.

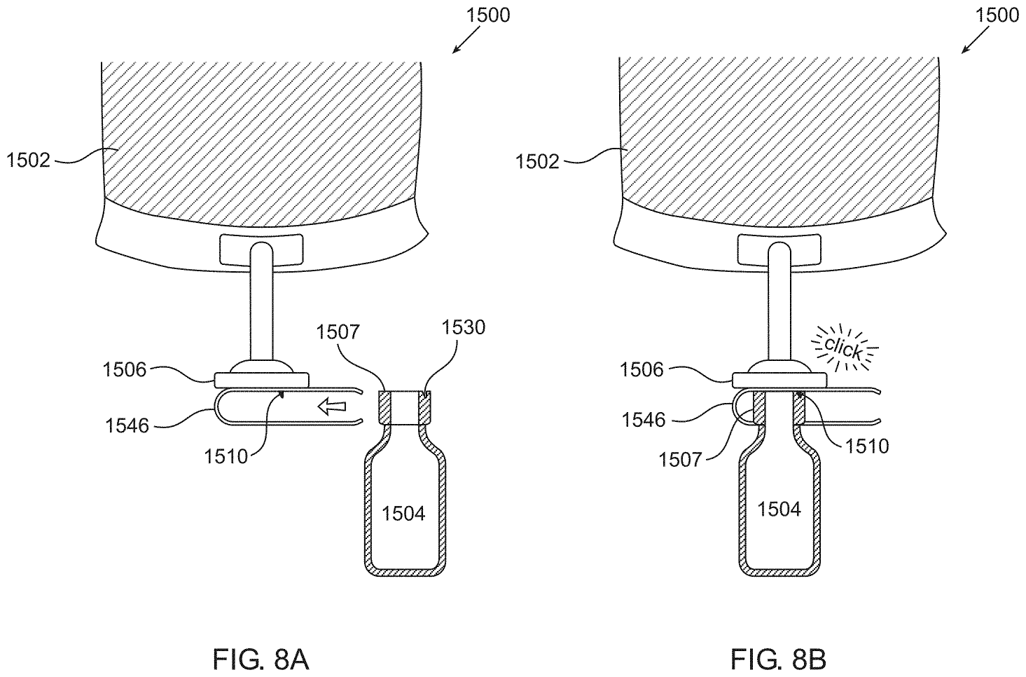

In FIGS. 8A-8B, system 1500 is shown, the system includes an engagement mechanism in the form of clamping rail 1546 which allows clamping of vessel 1504 to vessel 1502. The vessel can slide there along the clamping rail 1546. A permanent locking mechanism in the form of a dent 1530 on a surface of port 1507 which can lock into knob 1510 extending downwardly from a wall of clamping rail 1546 is provided. A port 1507 of vessel 1504 can be engaged within clamping rail 1546 and slide there along until reaching a longitudinal alignment with vessel 1502. A fluid passageway can be formed between the vessels following the engagement between the vessels and alignment thereof. When the dent 1530 meets knob 1510 a permanent engagement between vessels 1502 and 1504 is formed.

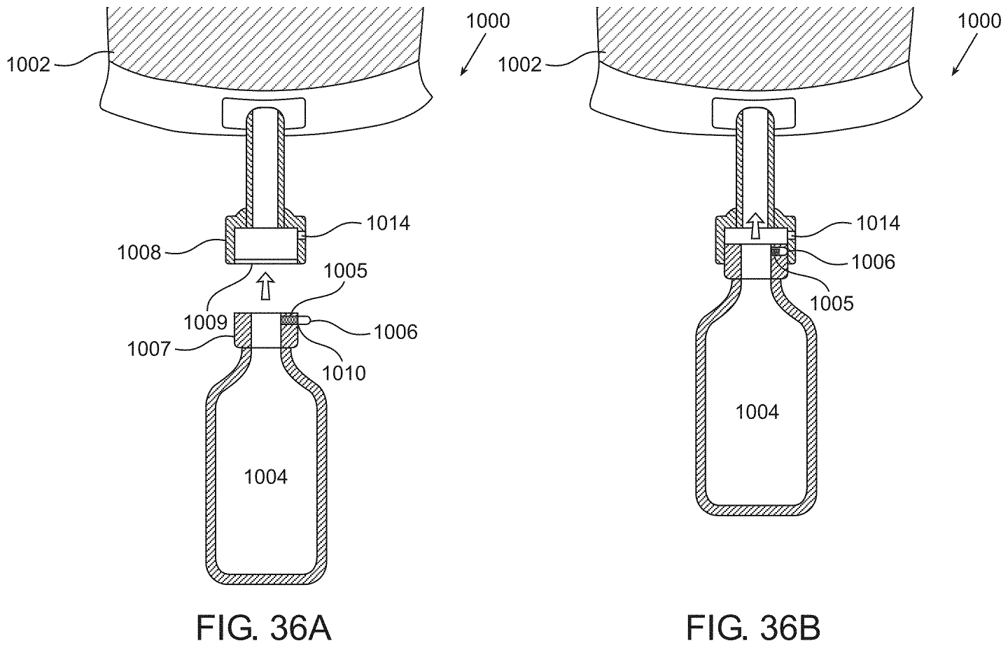

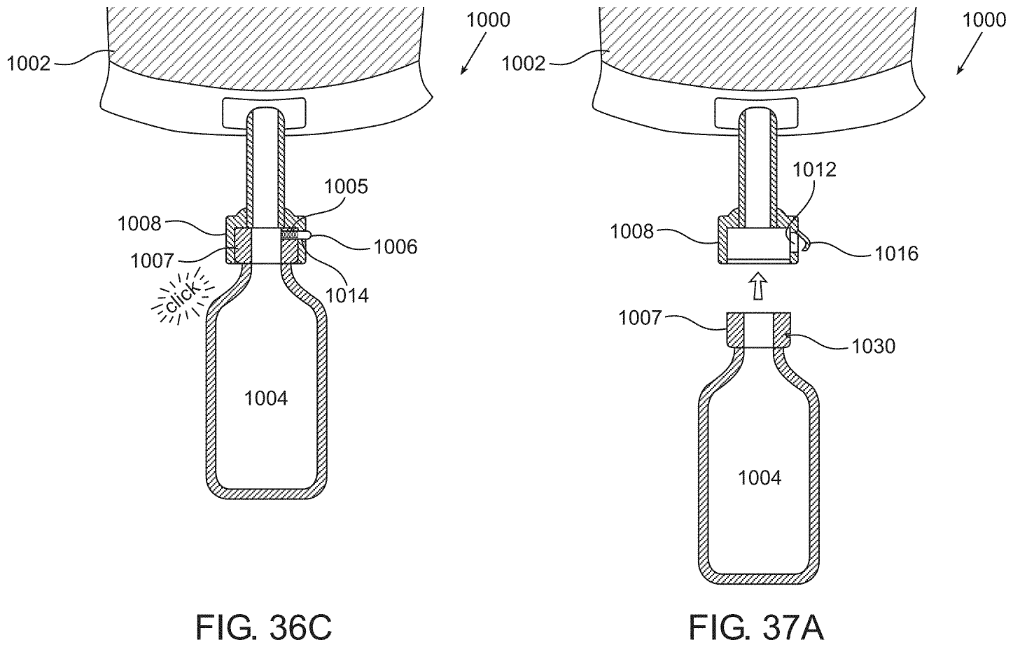

FIGS. 36A-36B illustrate a system 1000 with a locking mechanism, wherein the locking member is a pin 1006 loaded with spring 1005 and the securing member is a hole 1014. Pin 1006 is disposed within a dedicated lumen 1010 in a side wall of port 1007. The engagement between vessel 1002 and vessel 1004 is such that port 1008 includes an opening 1009 allowing port 1007 to be inserted within port 1008, optionally under a friction match. Spring 1005 allows pin 1006 to move between a retracted position (FIG. 36B) and an extended position (FIGS. 36A and 36C). FIG. 36B shows the pin 1006 that retracts such that it lies flat or flattens against a side wall of port 1007 upon sliding into port 1008 of vessel 1002. FIG. 36C shows the pin 1006 when extended through hole 1014.

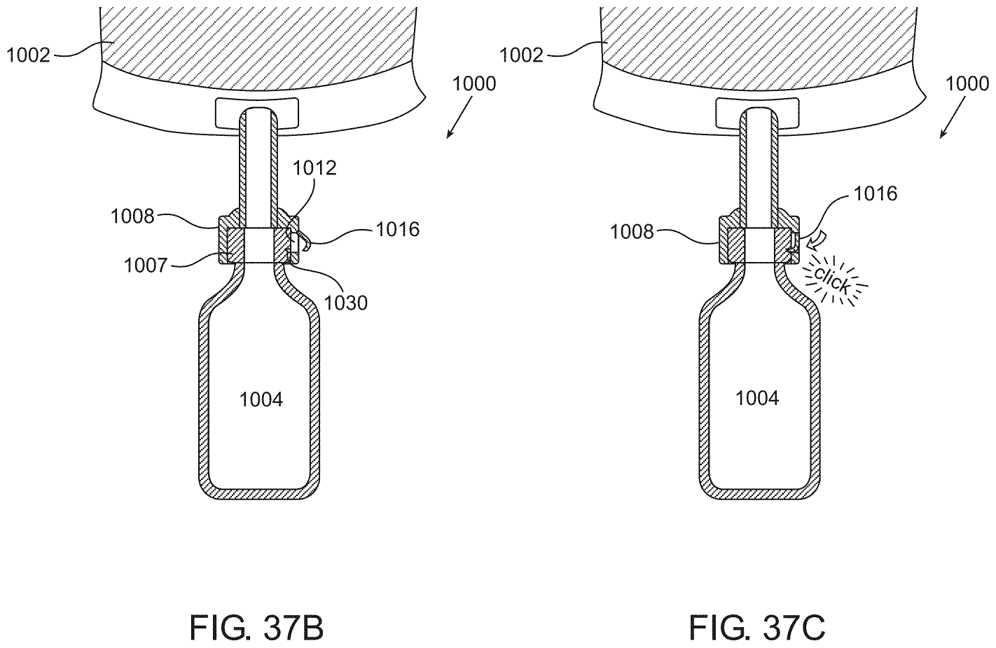

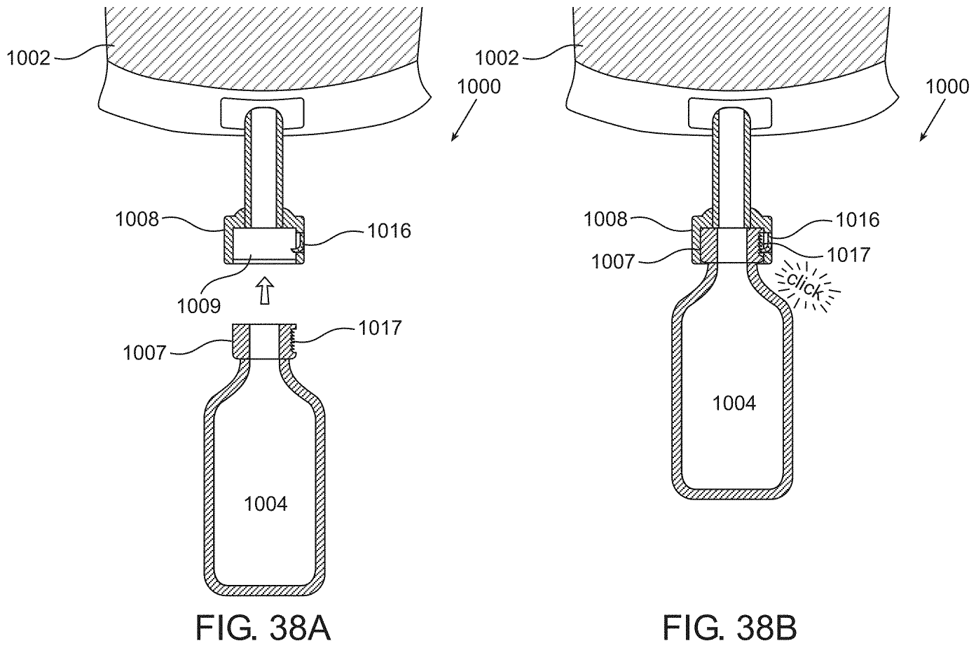

An alternative locking mechanism of system 1000 is shown in FIGS. 37A-38B. The locking member is hook 1016 and the securing member is dent 1030 (FIGS. 37A-37C) or a ratchet tooth (FIGS. 38A-38B). In the figures, hook 1016 disposed in a dedicated lumen 1012 in port 1008 and protrudes externally from the outer surface of side walls of port 1008. Dent 1030 or ratchet tooth 1017 disposed in an external surface of port 1007. The hook 1016 is moveable into and out of lumen 1012 and dent 1030, optionally, via a hinge (now shown). Hook 1016 may be pushed out to allow sliding of port 1007 into a lumen in port 1008 (FIG. 37A) and pushed into dent 1030 to allow locking the vessels 1002 and 1004 in an irreversible manner (FIGS. 37C and 38B). Optionally, hook 1016 may be disposed on an inner surface of port 1008 so as to prevent manual manipulation by a user. Vessel 1002 is shown as a bag but may be any vessel such as a container, a bottle, a connector, an infusion line, a vial, etc. Vessel 1004 is shown as a vial but may be any vessel such as a container, a bottle, a connector, an infusion line, a vial, etc.

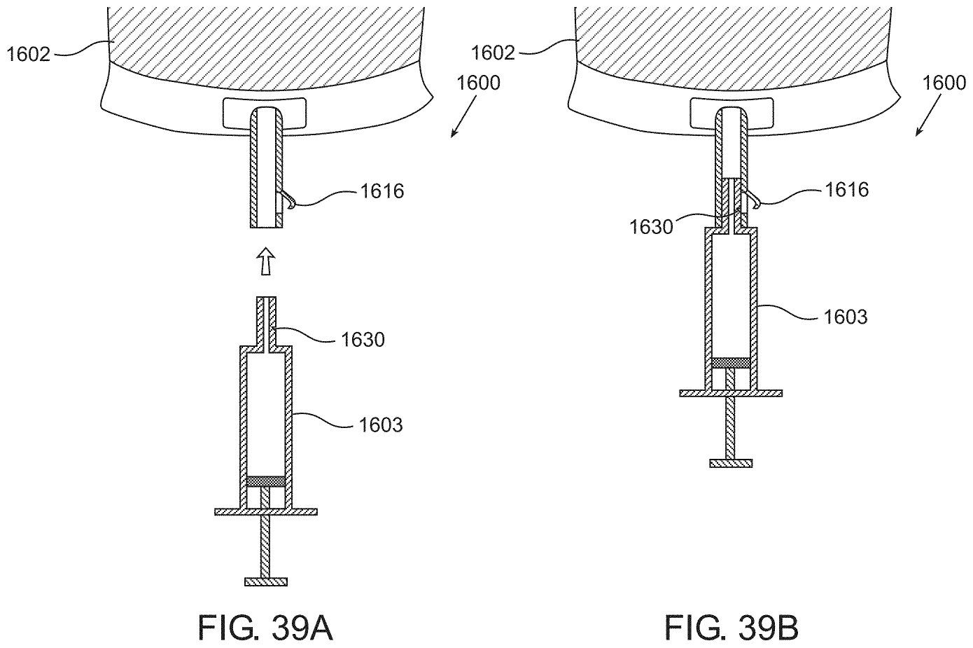

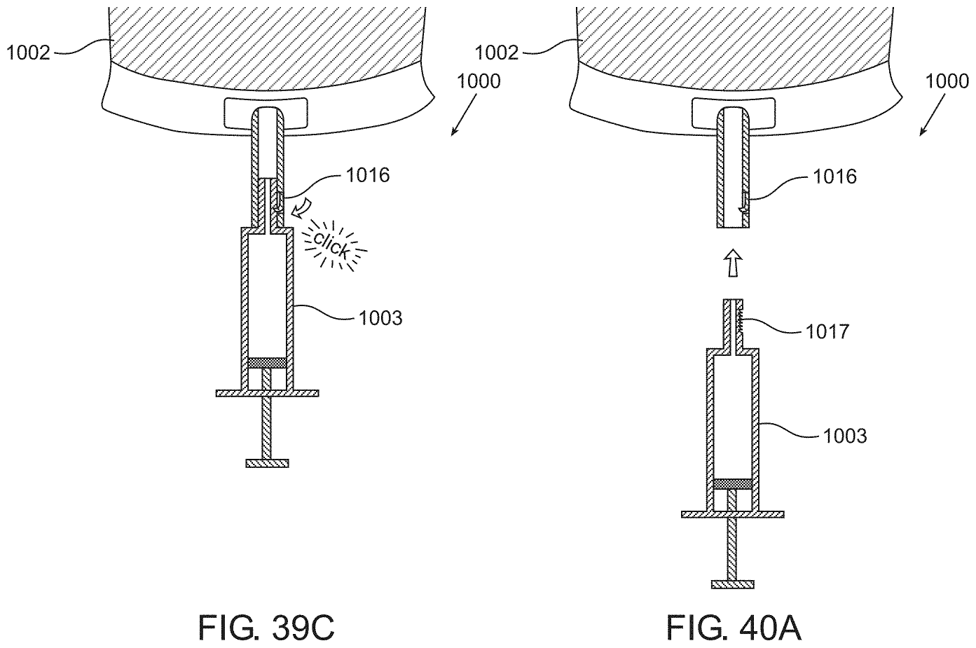

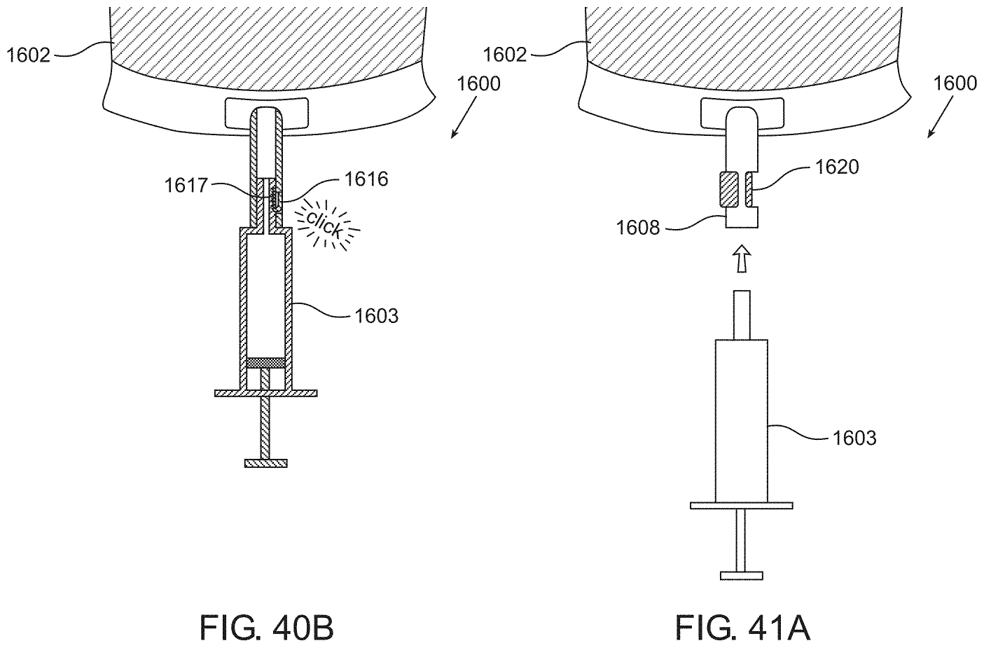

FIGS. 39A-39C illustrate system 1600 which includes a syringe 1603 configured to attach bag 1602. The locking mechanism is similar to the locking mechanism of FIGS. 37A-37C) and includes a hook 1616 and a dent 1630. An alternative securing member in the form of ratchet teeth 1017 is shown in FIGS. 40A-40B.

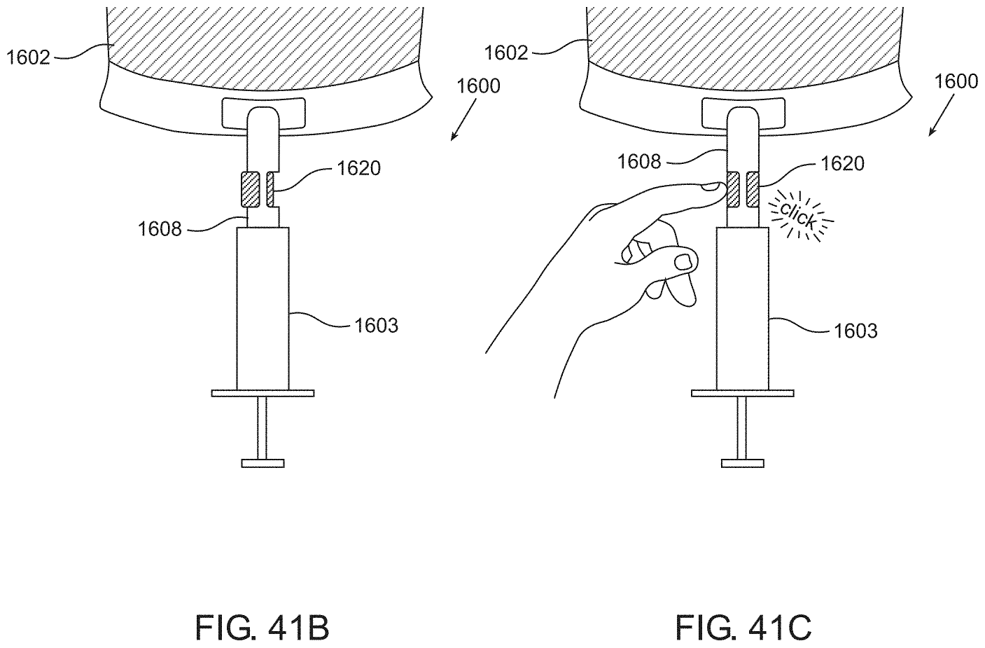

FIGS. 41A-41C illustrate a locking mechanism in the form of securing key 1620 disposed on a port 1608 of vessel 1602. The securing key is moveable in and out in port 1608 and may be optionally connected or loaded onto a spring (not shown) within port 1608. Sliding of vessel 1602 within or onto a port of vessel 1603 is afforded when securing key 1620 is in a retracted position (FIGS. 41A-41B). A locking of the vessels occurs when the securing key pushed to lock vessel 1602 (FIG. 41C).

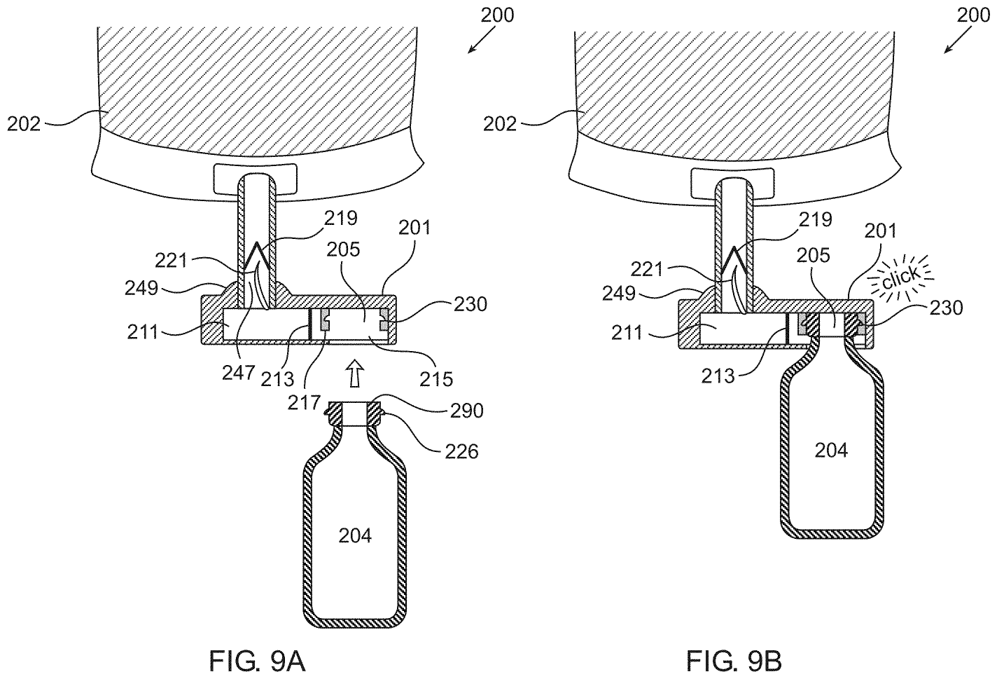

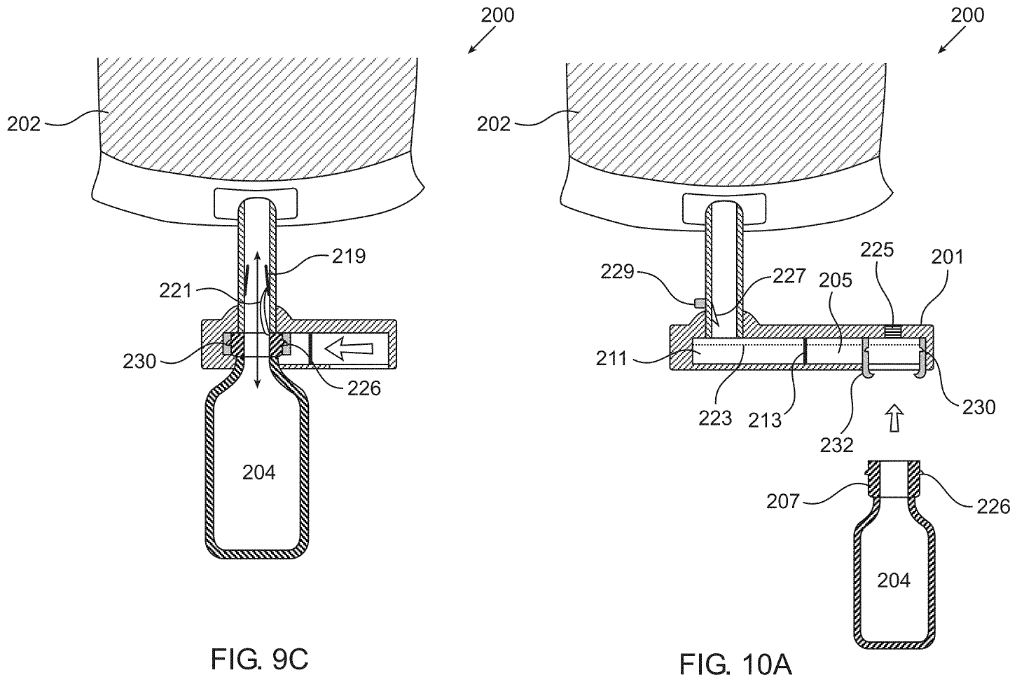

FIGS. 9-20 show various embodiments of decontamination devices as disclosed in the teachings of U.S. non-provisional patent application No. 16/100,840 filed on Aug. 10, 2018 titled Systems, Devices and Methods for Decontaminating Surfaces of Pharmaceutical Vessels and Pharmaceutical Administration Devices which is incorporated by reference as if fully set forth herein in its entirety.

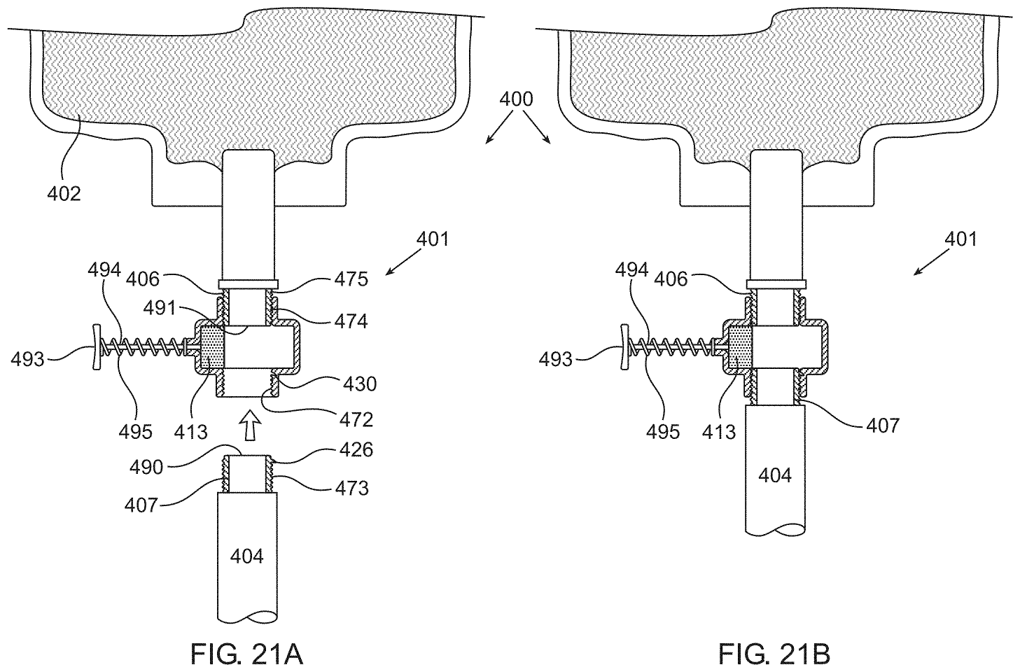

The following FIGS. 9-35 illustrate an irreversible or permanent locking mechanism between a device for decontaminating a surface of one or more medical vessels and a medical vessel. Various types of such decontamination devices are herein contemplated. Non-limiting examples refer to decontamination devices with a wiping member that is configured to wipe off the surface of the vessel. In one or more embodiments, the medical vessel is moveable within the device and when passing through the wiping member, being decontaminated (shown in FIGS. 9A-20). In alternative or additional embodiments, the wiping member itself is moveable within the device and when passing over a surface of a medical vessel decontaminates the surface(s) of the vessel (shown in FIGS. 21A-35).

FIGS. 9A-9C illustrate system 200 that includes a decontamination device wherein when one or more medical vessels when engaged with the device cannot detach. System 200 includes a first vessel, being an infusion bag 202 and a second vessel, being a vial 204. Various alternative vessel types could be used and are herein contemplated, such as a bottle, a vial, an infusion line, a connector, a filter, a manifold, a cartridge, etc. System 200 includes a housing 201 having a bottom, a top and side walls and enclosing a lumen. The housing 201 further optionally comprises vertical side walls 249 defining a top opening 247. Housing 201 further comprises a bottom opening 215. Top opening 247 is configured to receive vessel 202 and bottom opening 215 is configured to receive vessel 204. Openings 247 may be covered by a cover (not shown) providing a sealed system 200 prior to an engagement with vessel 204. Alternatively, or additionally opening 215 may be covered by a cover (not shown) providing a sealed system 200 prior to an engagement with vessel 204. The cover may be a frangible cover/seal. The frangible seal may be a thin plastic film.

Housing 201 is divided into two compartments, being a first non-sterile compartment 205 and second sterile compartment 211. A wiping member 213 is disposed within the housing 201, between first compartment 205 and second compartment 211. Housing 201 is constructed such to allow movement of second medical vessel 204, from a first position in first compartment 205 (shown in FIG. 9B) to a second position in second compartment (shown in FIG. 9C).

The wiping member 213 is configured to decontaminate the surface 290 of vessel 204 upon movement of the vessel 204 towards second compartment 211. Wiping member 213 extends between the top and bottom inner surfaces of housing 201, thus allowing a substantially isolated compartment 205 and 211. The housing 201 further includes an opening 215 sized and shaped to allow vessel 204 to attach and move within housing 201.

The housing 201 includes in first compartment 205 a connecting structure in the form of extensions 217. The connecting structure allows connection with a vessel, here vessel 204, via a locking mechanism and/or via an engagement mechanism. The vessel's locking mechanism in system 200 includes a locking member in the form of teeth 226 and a securing member in the form of dents 230 disposed in extensions 217 in first compartment 205. The attachment between vessel 204 and housing 201 is irreversible and made via teeth 226 that fits and locks into dent 230. As shown here, dent 230 is made part of extensions 217 which affixed to a top inner surface of housing 201. Nevertheless, alternative dent configurations and positions are contemplated and may be applicable. The attachment of vessel 204 to housing 201 may be accompanied with an audible sound, such as a “click” sound or any other sound heard at about the time, or at the time of engagement/locking of the vessels 202 and 204 (shown in FIG. 9B). In FIG. 9A, the vessels 202 and 204 are shown when spaced apart. In FIG. 9B, vessel 204 is attached to housing 201 via opening 215 and by tooth 226 that fits to connect to dent 230. In FIG. 9C, the vessel 204 is shown when sliding within housing 201 when attached to extensions 217. Once reaching second compartment 211, a fluid passageway can be formed between the vessels upon opening of valve 219 which may be a one way, or a two-way valve, and which is closed prior to any decontamination action made by wiping member 213. Valve 219 may be operable via valve unlocking mechanism 221 which allows opening of valve 219 at about the time, or at the time of longitudinal alignment of vessel 204 with vessel 202 (FIG. 9C). Optionally, system 200 is provided when housing 201 is connected already to, optionally integrally manufactured with, vessel 202. Extensions 217 are configured to slide between compartment 205 and compartment 211 and to move a surface 290 of vessel 204 between compartment 205 and compartment 211. In one or more embodiments, housing 201 may have more than 2 compartments. For example, 3 compartment, 4 compartments, and 5 compartments. According to this embodiment housing 201 may have more than one wiping members separating the compartments.

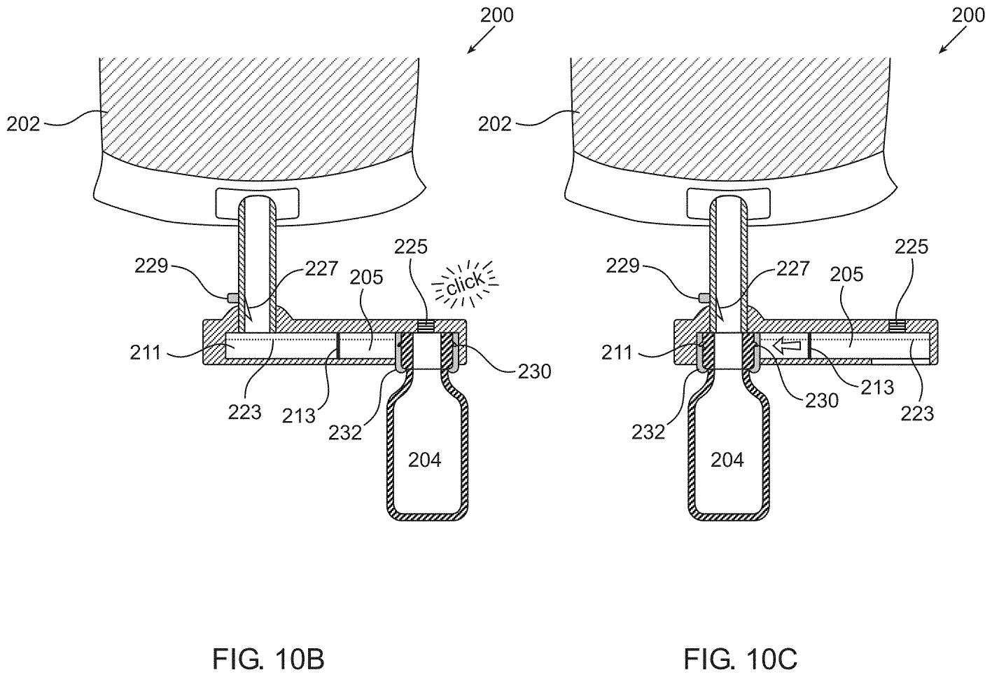

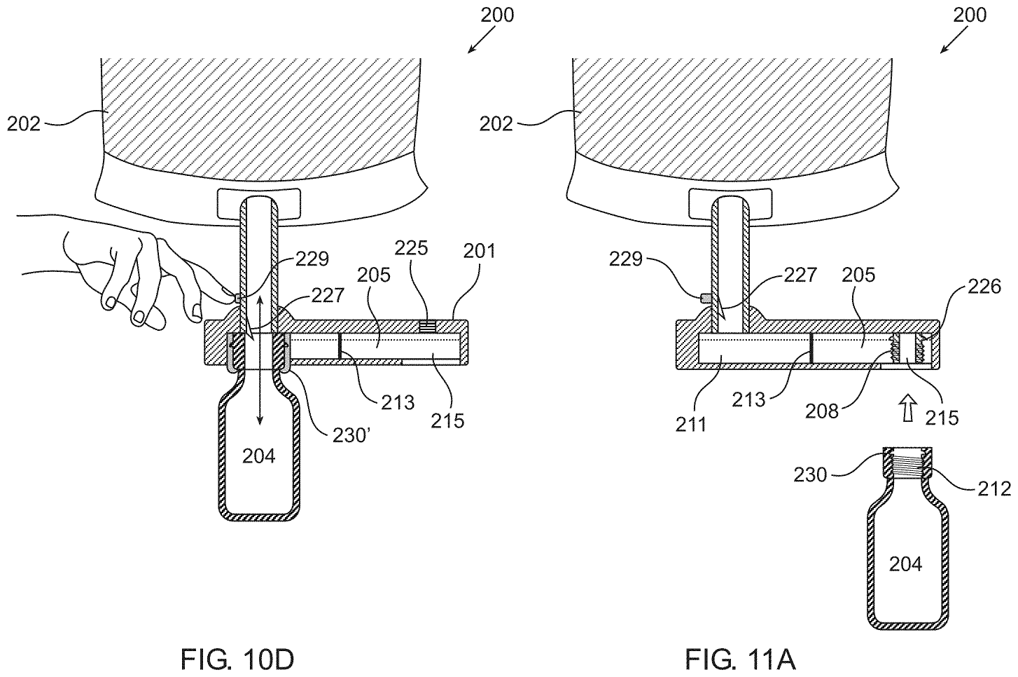

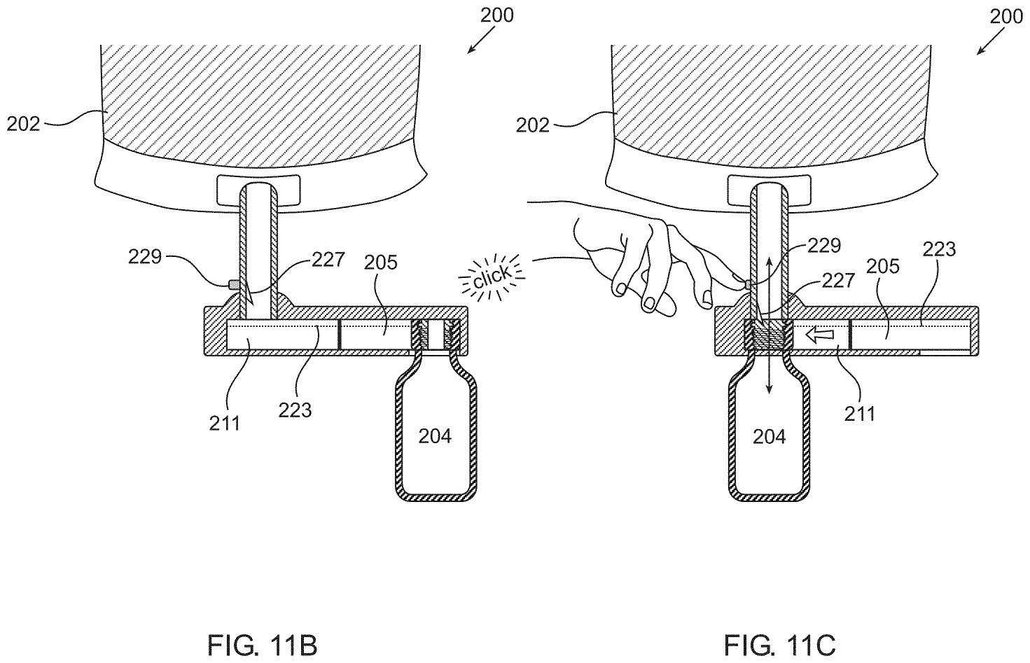

FIGS. 10A-10D illustrate system 200 wherein the engagement mechanism between the vessels 202 and 204 is made via connecting structure in the form of clamping member 232. The clamping member 232 can clamp port 207 of vessel 204. Teeth 226 disposed to outwardly protrude from the sides of port 207 are configured to cling/lock into dents 230 of clamping member 232. The system 200 further includes a filter 225 to allow for releasing air from the housing 201 during movement of vessel 204. Accordingly, air pressure that can be built up during movement of the vessel can be prevented. Filter 225 is located and devised so as to allow the pressurized air to pass there through, without allowing any contaminants inside. Once irreversibly attached (FIG. 10B), vessel 204 slides along the housing 201 via rail 223 until reaching compartment 211 when the surface of the vessel 204 had been decontaminated by wiping member 213 (FIG. 10C). A piercing member 227 can then pierce a seal or cover (not shown) of second medical vessel 204 upon a user actuating piercing member actuator 229. Piercing member 227 may be a needle, optionally, a hollowed needle, but various types of piercing members are contemplated, such as members that possess a sharp edge. Alternative engagement mechanisms and locking mechanisms of system 200 are shown in FIGS. 11A-11C, the engagement between vessels 202 and 204 is made via a thread-luer mechanism. A male threaded connecting structure 208 extending from a top wall within first compartment 205 is configured to be coupled or engaged with a female luer 212 within port 207. The male thread 208 includes a protruding tooth 226 which can be coupled to or fit a complementary dent 230. The thread 208-female luer 212 mechanism allows an engagement between the vessels, whereas coupling of teeth 226 to dent 230, provides an irreversible locking of vessel 204 to housing 201. The vessel 204 can then slide along the housing 201 until reaching second compartment 211 and establishing a fluid communication or passageway between the vessels 202 and 204.

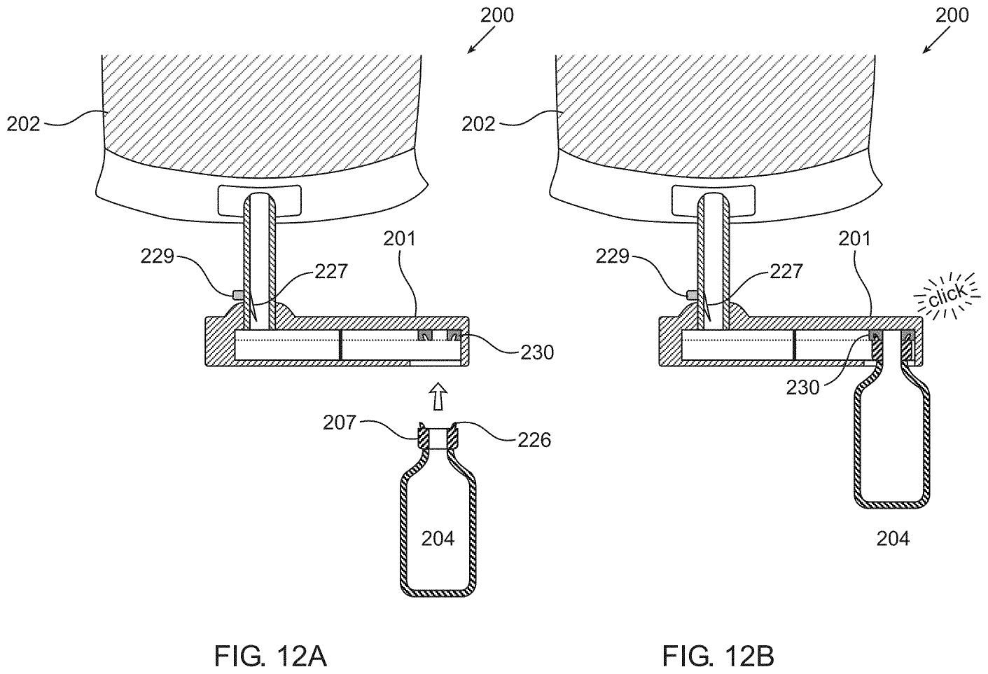

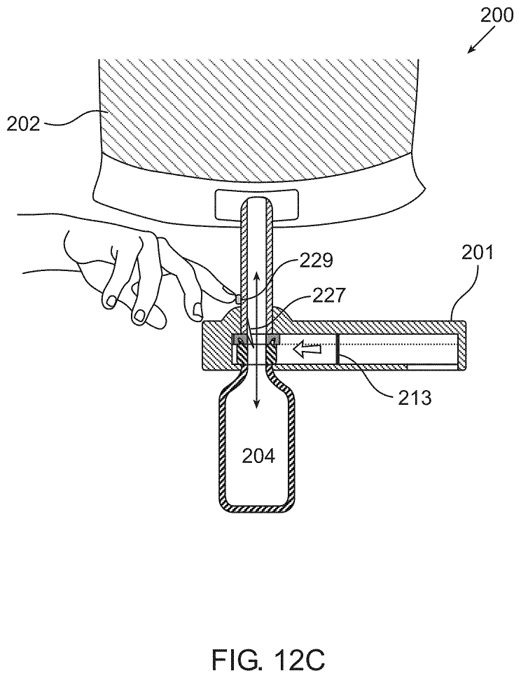

In FIGS. 12A-12C teeth 226 are located on and outwardly protrude from the surface of port 207. The housing includes a connecting structure having minimized built-in dents 230 that can receive and lock therein the teeth 226, obtaining an irreversible engagement between the vessel 204 and housing 201. The portion (surface) of vessel 204 can then slide within the housing and move to second compartment 211. Upon a user actuating piercing member 227 via piercing member actuator 229, a fluid passageway can be obtained between vessels 204 and 202.

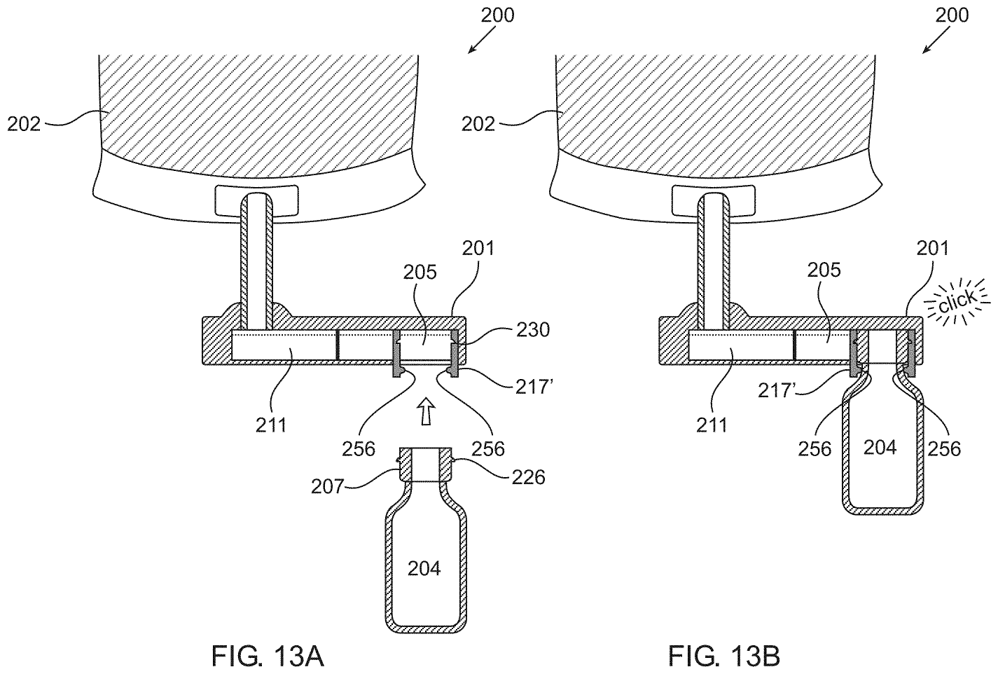

FIGS. 13A-13B illustrate an engagement mechanism that is similar to the engagement mechanism shown in FIGS. 9A-9C with the exception that extensions 217′ are elongated and include circumferential wipers 256 that outwardly protrude and allow wiping the sides of port 207 when engaging with housing 201. Optionally, the circumferential wipers 256 can also assist to the engagement between vessel 204 with housing 201. Accordingly, circumferential wipers 256 allow wiping off side walls of port 207 under great friction, or under a friction sufficient to decontaminate the sides of port 207.

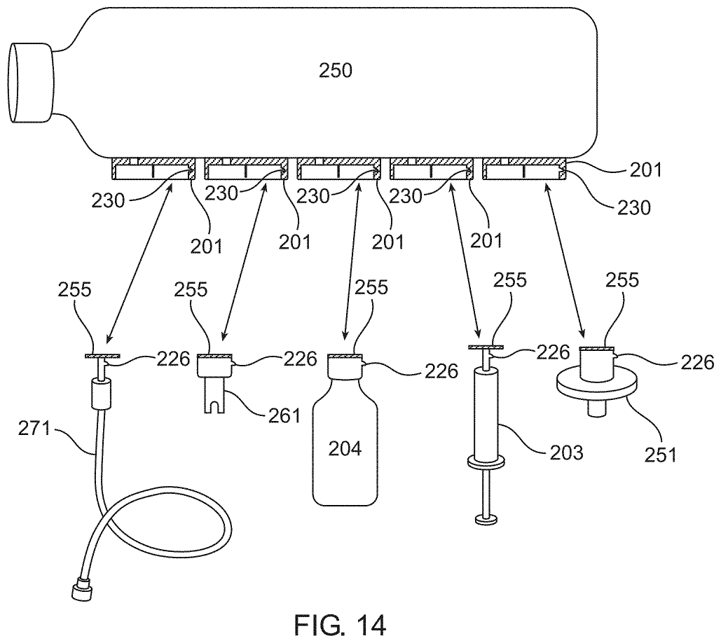

FIG. 14 illustrates a vessel 250 which is connected to a plurality of housings 201, each of which configured to connect a medical vessel, such as filter 251, syringe 203, vial 204, connector 261 and infusion line 271. Vessel 250 can therefore connect a plurality of medical vessels in a decontaminated manner. In one or more embodiments, housings 201 may be surface mounted to vessel 250. Optionally, housings 201 may be flush mounted (not shown) to vessel 250. Optionally, housings 201 may abut a wall of vessel 250. One or more of a tooth 226 is disposed about a surface of the port of vessels 251, 203, 204, 261, and 271 to allow an irreversible attachment to housing 201 via a complementary dent 230. The vessels 251, 203, 204, 261, and 271 are further sealed by cover/seal 255. The cover/seal may be either removed when the vessel is engaged with housing 201 or pierced via a piercing member as demonstrated herein in the above figures. Optionally, cover/seal 255 may be a frangible seal, for example a film, optionally a thin plastic film.

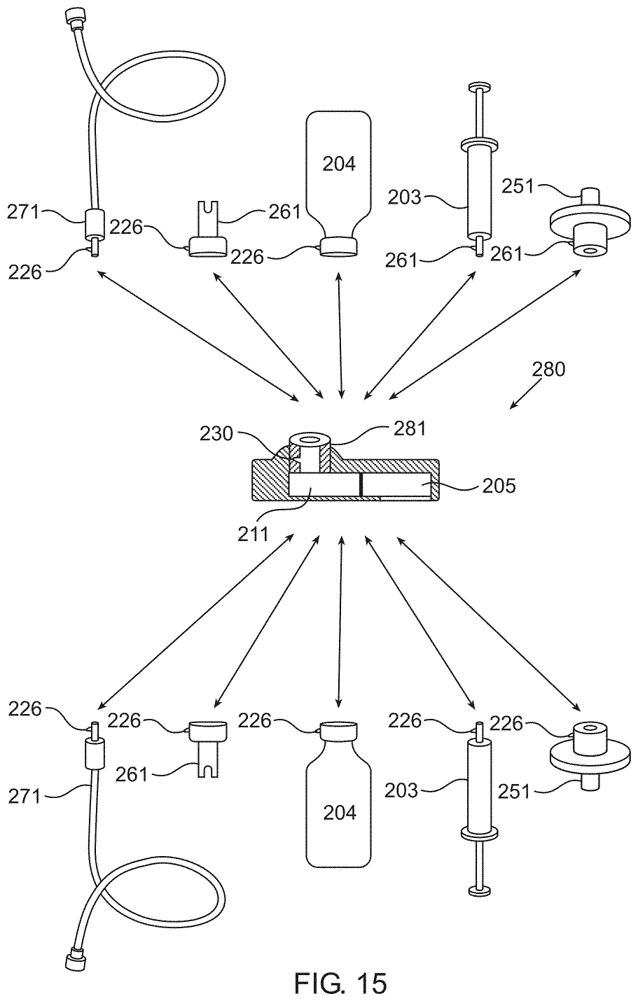

FIG. 15 illustrates a connector 280 which includes a vessel's locking mechanism in the form of teeth 226/261 and dent 230. The dent 230 is positioned in this case in a port 281 of second compartment 211. The connector 280 may be provided when already coupled with first medical vessel. Alternatively, the connector 280 can be provided as a separate unit not connected to any medical vessel. Optionally, a first medical vessel can be coupled with connector 280 in a sterile manner, optionally, within a sterile chamber or room. Optionally, a first medical vessel can be coupled and/or integrally attached and/or manufactured with connector 280. Optionally, a first medical vessel can form a unitary structure with connector 280 and be provided to consumers as such. The connector 280 is further configured to be coupled to various types of second medical vessels. The first and/or second medical vessel may be selected from a filter 251, a syringe 203, a vial 204, a connector 261, and an infusion line 271. Other vessels are also contemplated such as a manifold, a connector, a bag, a buretrol, etc.

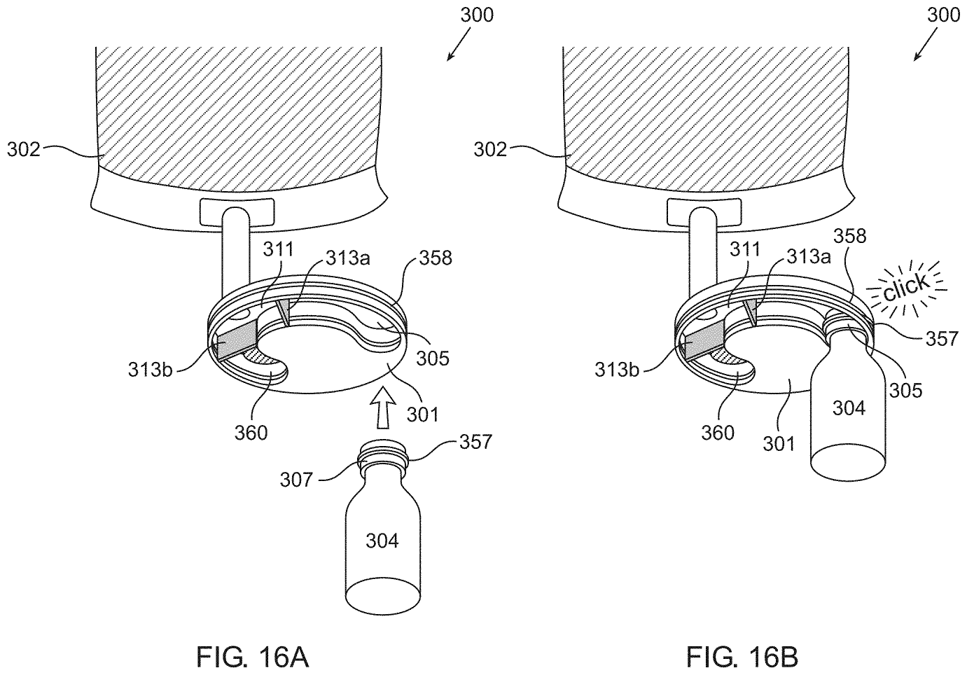

FIGS. 16A-16B illustrate system 300 which includes a housing 301 with three compartments being: first compartment 305 configured for allowing an engagement with vessel 304, second compartment 311 for allowing a fluid passageway between two vessels and third compartment 360. System 300 further includes a first wiping member 313a disposed between first compartment 305 and second compartment 311 and a second wiping member 313b disposed between second compartment 311 and third compartment 360. The system 300 includes an irreversible/permanent locking mechanism that affords a secure connection of vessel 304 to housing 301. The locking mechanism here includes a circumferential protruding bangle 357 which extends from the sides of port 307 and which can be coupled to and locked within channel 358 of housing 301 of the decontamination device. The channel 358 in housing 301 is circumferential along a length of an outer wall and continuously curves internally. Upon engagement of vessel 304 to housing 301 a permanent connection is formed and vessel 304 cannot detach from housing 301. Optionally, bangle 357 is retractable and flattens against a wall of port 307 when the vessel is being pushed inside housing 301 and extendible within channel 358 when the bangle 357 positioned parallel to the channel 358.

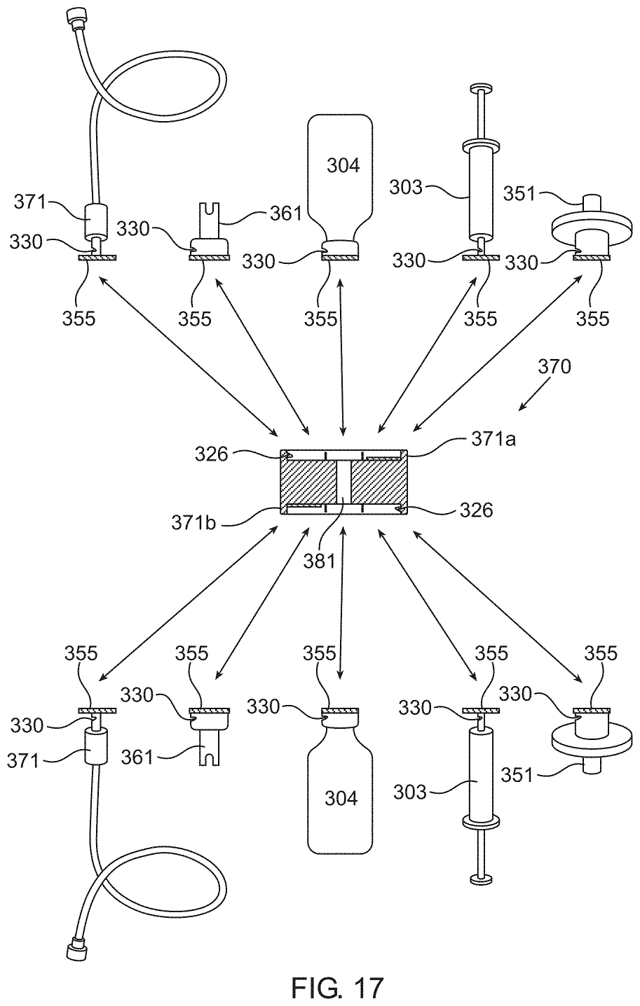

FIG. 17 demonstrates a connector 370 which includes two housing elements, being top housing 371a and bottom housing 371b disposed one above the other and integrally connected. Housing 371a is arranged to decontaminate a vessel which can be a filter 351, a syringe 303, a vial 304, a connector 361, or an infusion line 371. The connector 370 has a permanent locking mechanism in the form of tooth 326 and dent 330. The connector 370 has a conduit 381 formed between the middle compartments of top housing 371a and bottom housing 371b, allowing for a fluidic communication between two vessels. The vessels may be provided with a seal or a cover 355 which may be released in first compartment of the housings 371a and 371b. Top housing 371a and/or bottom housing 371b may be provided with a seal or a cover. The seal or cover may be frangible, for example a thin film. The film may be a plastic film.

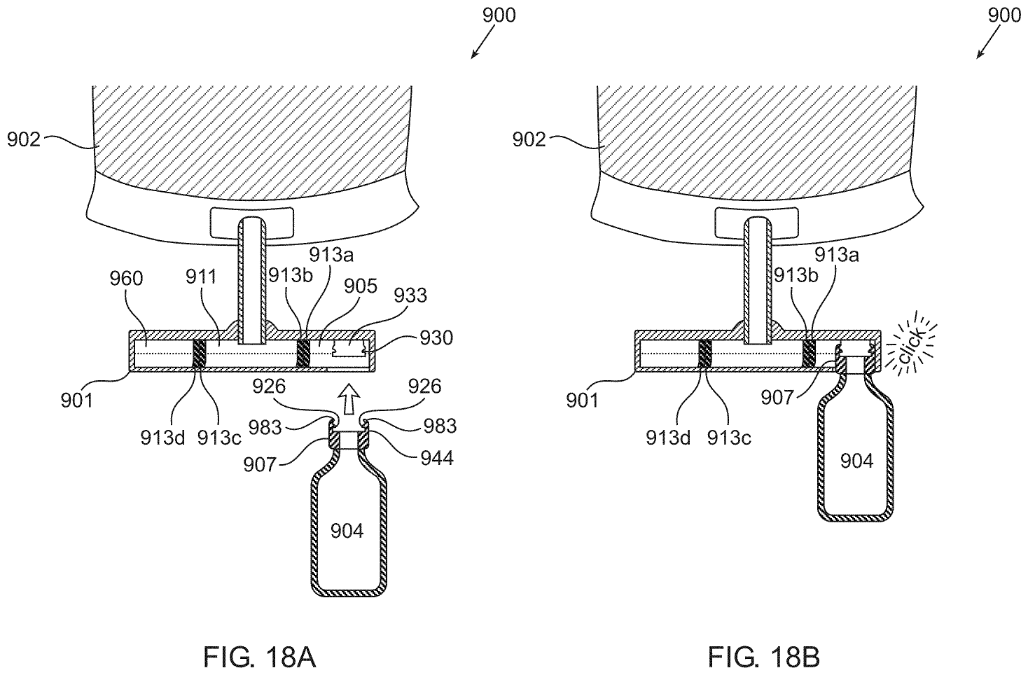

FIGS. 18A-18B demonstrate system 900 with two pairs of double wipers 913a, 913b, 913c and 913d, wherein in each pair the wipers being in contact with each other. The system 900 includes three compartments, being first compartment 905, second compartment 911 and third compartment 960. The system 900 includes a permanent locking mechanism wherein the securing member includes a connecting structure in the form of circular body 933 disposed in first compartment 905. Vessel 904 can be coupled and locked into first compartment 905 via teeth 926 which in this case disposed internally within vertical side walls 944 extending from the top wall of the port 907 and which surround a lumen in port 907. The circumferential wall 944 ends with fasteners 983 configured to engage body 933. Teeth 926 can fit and attach dent 930 in body 933. A click sound may be produced upon or at about the time of engagement/locking between vessel 904 and housing 901 to alert a user that and engagement/locking has occurred.

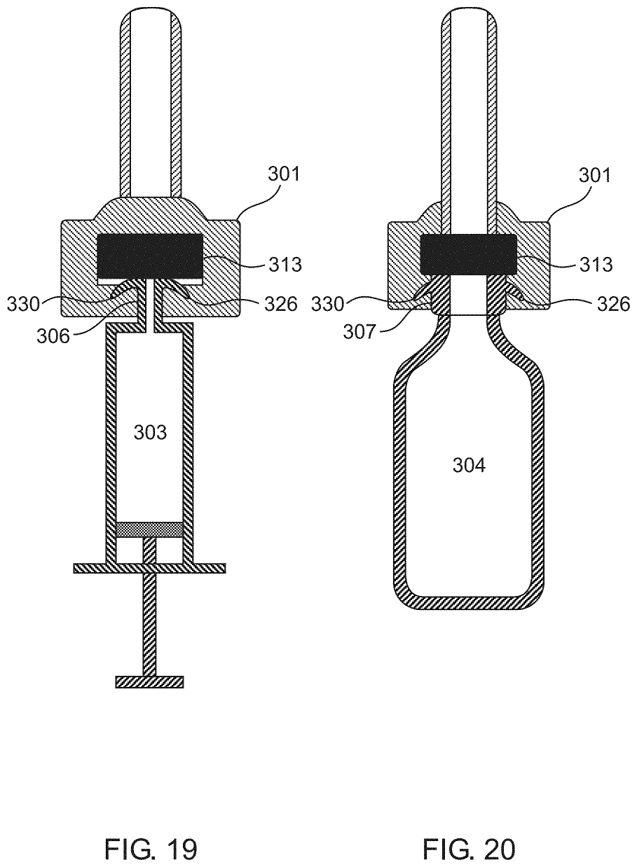

FIGS. 19-20 are side cross section views of a syringe 303 and vial 304 when permanently engaged with housing 301 via teeth 326 which fits to dent 330. The teeth 326 protrudes from the surface of the syringe's port and the vial's port, optionally allowing the syringe 303 and vial 304 to slide there along housing 301. Wiping member 313 is shown when wiping off a surface of the syringe 303 and vial 304. As shown herein, teeth 326 may protrude outwardly from a side wall of port 307 (FIG. 20). Alternatively, teeth 326 may extend from the top wall of a port, such as port 306 (FIG. 19).

The following FIGS. 21-35 illustrate an irreversible locking mechanism between a device for decontaminating a surface of one or more medical vessels and a medical vessel. The decontamination is made via a wiping member that is moveable within the device such that when passing over a surface of a medical vessel, decontaminates the surface(s) of the vessel.

FIGS. 21-35 show various embodiments of decontamination devices as disclosed in the teachings of U.S. non-provisional patent application Ser. No. 16/100,964 filed on Aug. 10, 2018 titled Decontamination Device for Pharmaceutical Vessels which is incorporated by reference as if fully set forth herein in its entirety.

FIGS. 21A-21B illustrate a system 400 for decontaminating medical vessels, which has a moveable wiping member 413. The system 400 includes a housing 401 that allows connection to a first medical vessel 402 and a second medical vessel 404 and within which a wiping member 413 is disposed. Wiping member 413 is configured to move across the housing 401 and to thereby decontaminate surfaces 490 and 491 of vessels 404 and 402, respectively. Wiping member 413 is manipulated from the exterior to move across the housing 401 by a wiping member actuator having a form of an actuator/handle 493. A transfer mechanism in the form of a rod 494 loaded by a spring 495 is configured to transfer a force to move the wiping member 413 within the housing 401. Vessel 402 and/or 404 may be provided when already attached, optionally integrally manufactured with, or when not connected to housing 401. The engagement of vessel 404 with housing 401 may be via internal threads 472, being configured for cooperating with external threads 473 on vessel 404. The engagement of vessel 402 with housing 401 may be via internal threads 474, being configured for cooperating with external threads 475 on vessel 402. An externally protruding tooth 426 on an end of port 407 is provided to allow a permanent engagement with housing 401 when the tooth 426 fits to lock dent 430 in housing 401.

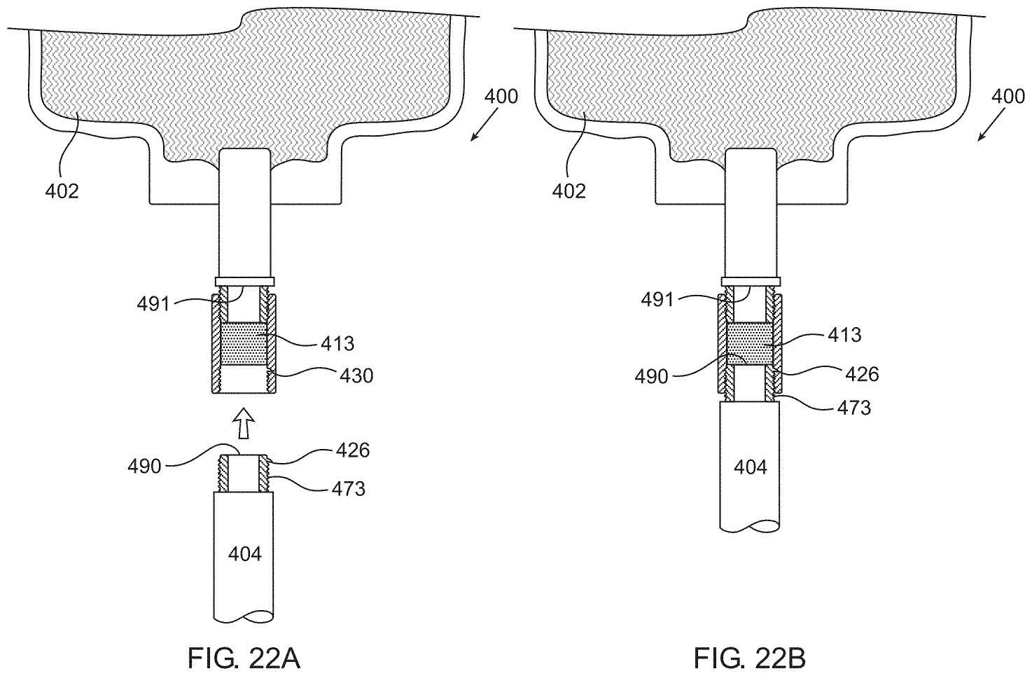

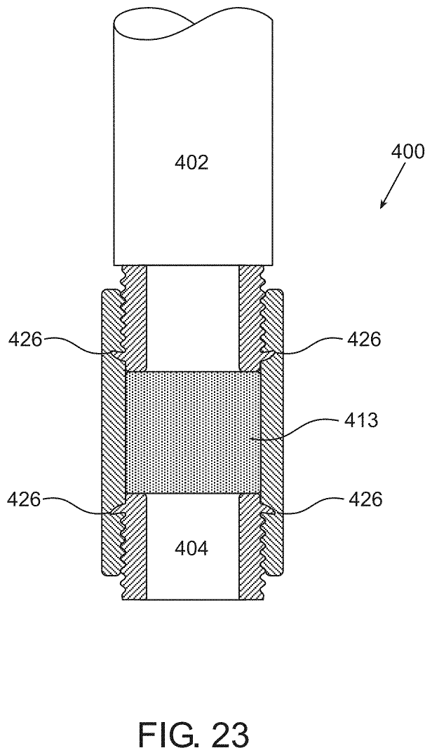

FIGS. 22A-22B show a cross sectional side view of system 400. Wiping member 413 may expand to cover the entire surface 490 and/or 491 of vessels 404 and/or 402, forming a hermetic seal between wiping member 413 and surfaces 490 and/or 491. Teeth 426 may be locked within complementary dent 430. Although a single tooth 426 and dent 430 are shown, but a greater number of teeth and complementary dents are herein contemplated. For example, FIG. 23 demonstrates yet another embodiment of system 400 which includes two teeth 426 in vessel 402 and two teeth 426 in vessel 404.

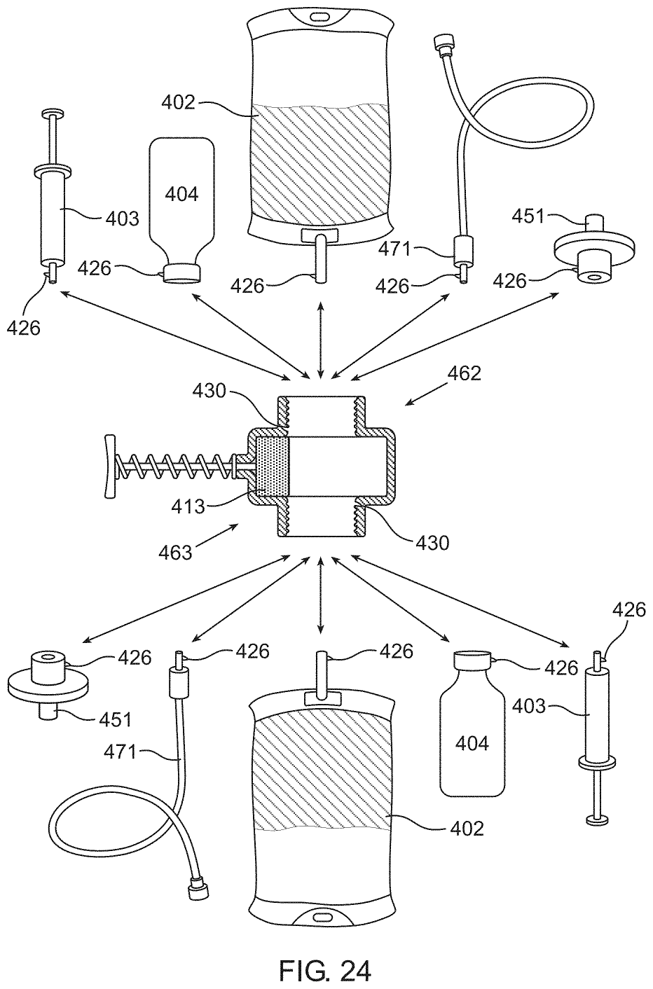

Reference is now made to FIG. 24 which illustrates the herein disclosed system 400 that may allow an engagement and decontaminated fluid passageway between two vessels. The system 400 includes a top port 462 and a bottom port 463 each can be engaged with various types of vessels, such as syringe 403, bottle/vial 404, infusion bag 402, infusion line 471 and filter 451. Other vessels are also contemplated such as a manifold, a connector, a buretrol, etc.

Optionally, one or both ports 462 and 463 is attached to or integrally attached in a permanent or fixedly manner to the vessel(s) via teeth 426-dent 430 mechanism.

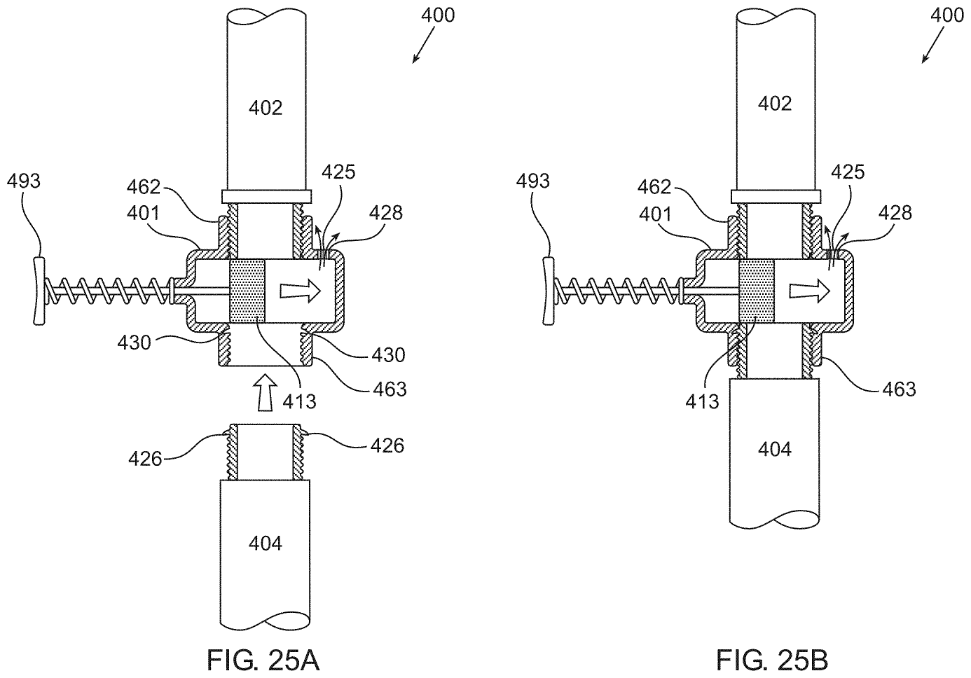

Reference is now made to FIGS. 25A-25B which illustrates system 400 when further comprises a filter 425 disposed in an aperture 428 in the housing 400. Filter 428 is configured to allow for releasing air from the housing 401 when actuating the wiping member 413. Accordingly, air pressure that can be built up during movement of wiping member 413 can be prevented. Filter 428 is located and devised so as to allow the pressurized air to pass there through, without allowing any contaminants inside. Filter 425 may have a porosity of about 0.22 microns or less. Optionally, filter 425 may be a valve (e.g., a one-way valve allowing air to pass out of housing 401 while preventing ambient or environmental air from entering the housing 401). Optionally, one or both ports 462 and 463 is attached to or integrally attached in a permanent or fixedly manner to the vessel(s) via teeth 426-dent 430 mechanism.

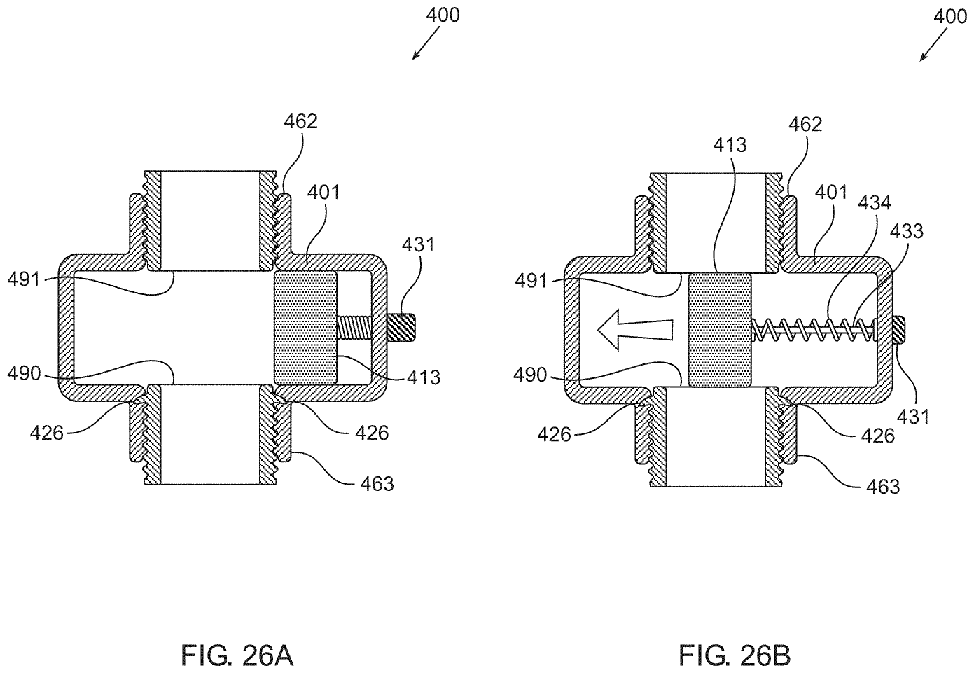

Referring now to FIGS. 26A-26B, an alternative actuator of system 400 is shown which having the form of button 431 attached to spring 434 loaded rod 433. The actuator 431 is actuated to manipulate sliding of wiper member 413 across housing 401 and wipe surfaces 490 and and/or 491 by pushing thereof. Optionally, one or both ports 462 and 463 is attached to or integrally attached in a permanent or fixedly manner to the vessel(s) via teeth 426-dent 430 mechanism.

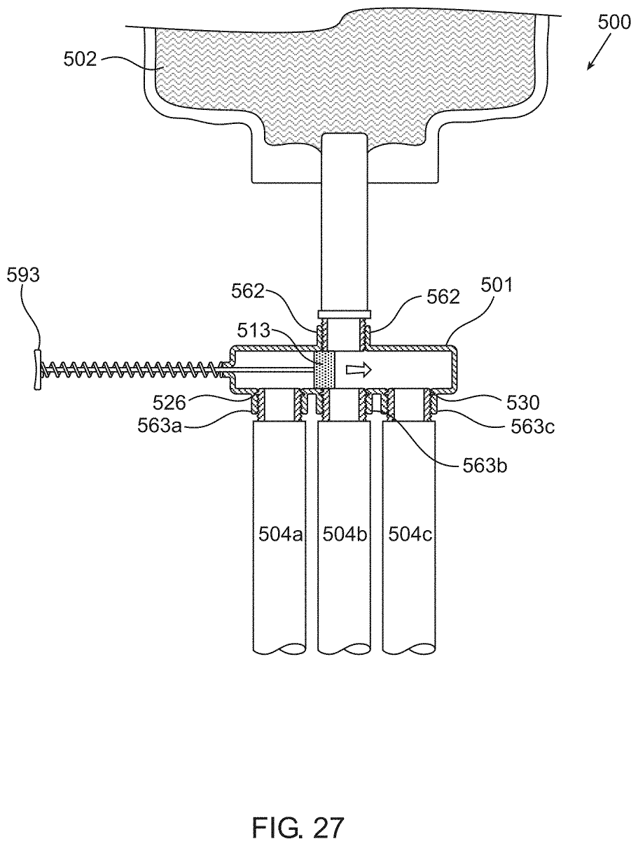

Reference is now made to FIG. 27 which illustrates a further exemplary system 500 wherein a housing 501 is made wide and contains three bottom ports 563a 563b, and 563c. The ports 563 include internal dents 530 which can lock vessels 504 via teeth 526. This way, three vessels 504 can be locked to housing 501 and can have their surfaces partially or entirely wiped off at about the time, sequentially, or in one action, by wiping member 513, when actuated by actuator 593. Optionally, each of the plurality of ports 563 provides airtight engagement between the housing 501 and vessels 504.

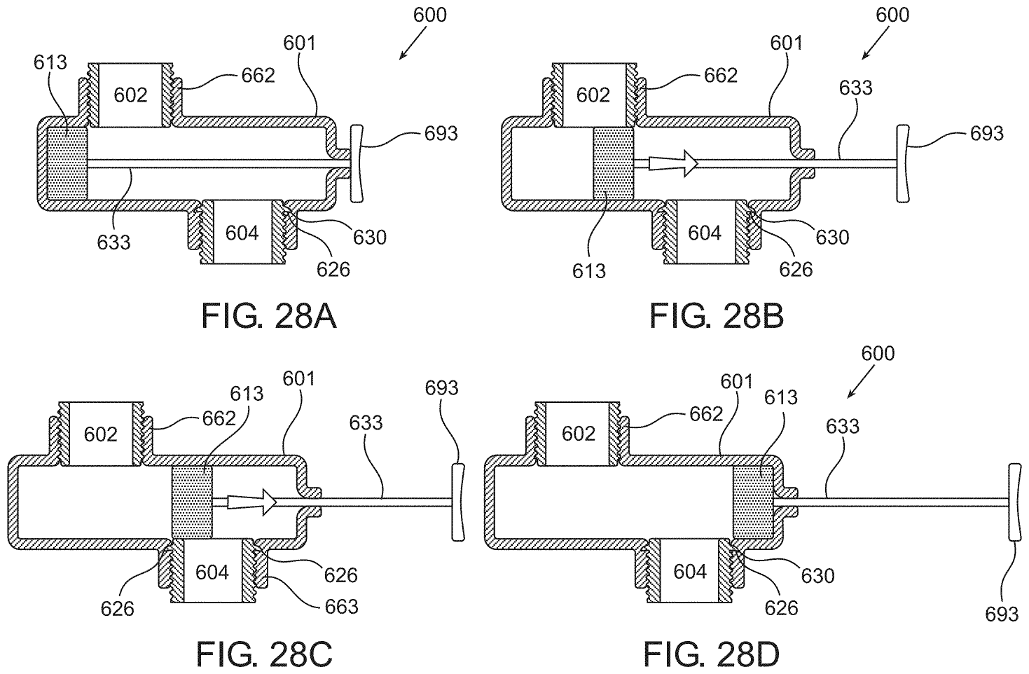

Referring to FIGS. 28A-28D, a further exemplary system 600 for decontaminating vessels is shown. System 600 includes housing 601 with ports 662 and 663. In this case ports 662 and 663 are shifted from each other and lie in parallel longitudinal axes. Ports 662 and 663 are configured to connect vessels 602 and 604, respectively. The connection is permanent via teeth 626 that lock into dents 630. The system 600 further includes an actuator 693 being a handle connected to rod 633, but this time without a spring. The actuator 693 is operable to allow movement across the housing 601 by an axial pulling motion. A fluid passageway between the vessels 602 and 604 can be formed upon engagement and locking of the vessels into housing 601.

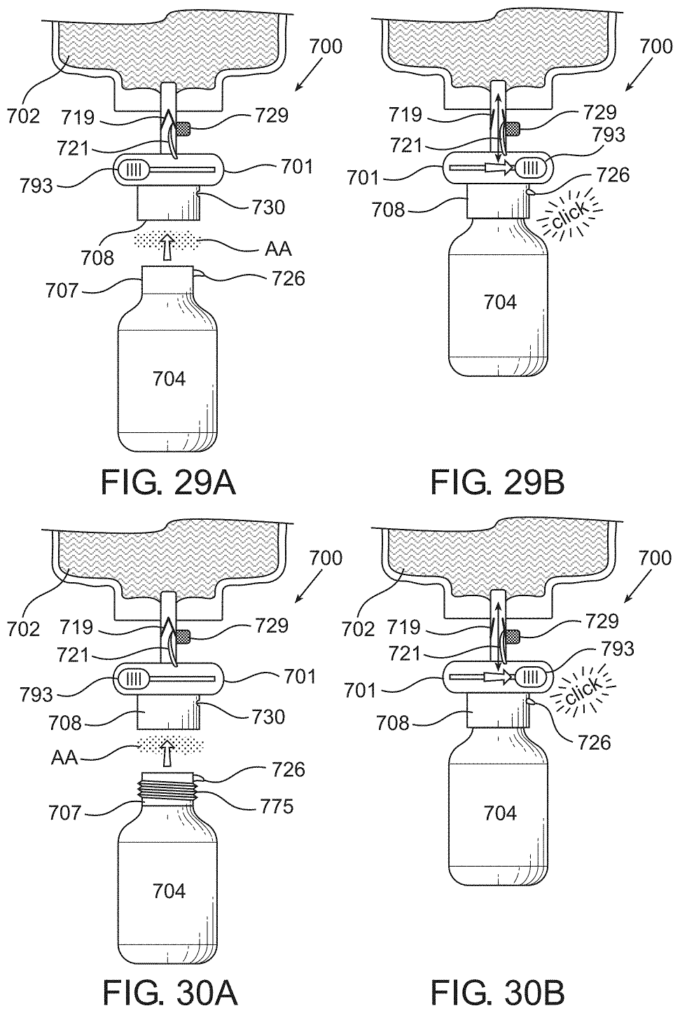

FIGS. 29A-29B shows system 700 which includes a port 708 with vertical walls defining an opening of housing 701. Port 708 is configured to allow coupling of vessel 704 to housing 701. Ambient air (AA) surrounding the surface of vessel 704 is wiped off by a wiping member (not shown) positioned in the interior of housing 701. A tab 793 may be used to actuate the wiping member. System 700 further includes a valve 719, optionally a one way, or a two-way valve, that is closed prior to any decontamination action made by wiping member. Valve 719 may be operable also via valve actuator 729 which actuates valve unlocking mechanism 721. Valve 719 is shown closed in FIG. 29A and opened in FIG. 29B. A fluid passageway is formed between vessel 702 and 704 following the movement and decontamination action of wiping member. The locking mechanism in this system includes a retractable tooth 726 and a hole 730. Vessel 704 is pushed upwards towards port 708 of housing 701, the retractable tooth 726 retracts into port 707 of vessel 704 to allow coupling of port 707 to port 708. When retractable tooth 726 aligns with hole 730 the retractable tooth 726 is positioned within and/or through hole 730 as shown in FIG. 29B thus providing a permanent and/or irreversible lock/engagement between vessel 704 and housing 701. Vessel 704 is shown as a vial but any vessel may replace vessel 704 such as a container, a syringe, a container with a plunger, a connector, an infusion line, a filter, a buretrol, etc. In FIGS. 30A-30B system 700 is shown when further comprising an engagement mechanism allowing engagement between vessel 704 and system 700 via a thread 775 disposed on vessel 704 that is complementary to a thread surrounding the internal of port 708 of system 700 (not shown). Vessel 704 is shown having a thread 775 and a retractable tooth 726. Vessel 704 is shown as a vial, however, any vessel may replace vessel 704 such as a container, a syringe, a container having a plunger, a buretrol, a connector, an infusion line, a filter, etc. Retractable tooth may move between a retracted position wherein the tooth 726 is disposed within port 707 of vessel 704 and an extended position as shown in FIGS. 29A-30B. Retractable tooth may be pushed out/kept in the extended position by a spring (not shown) disposed inside port 707 of vessel 704. The spring may be configured to allow retractable tooth 726 to move between a retracted position and an extended position.

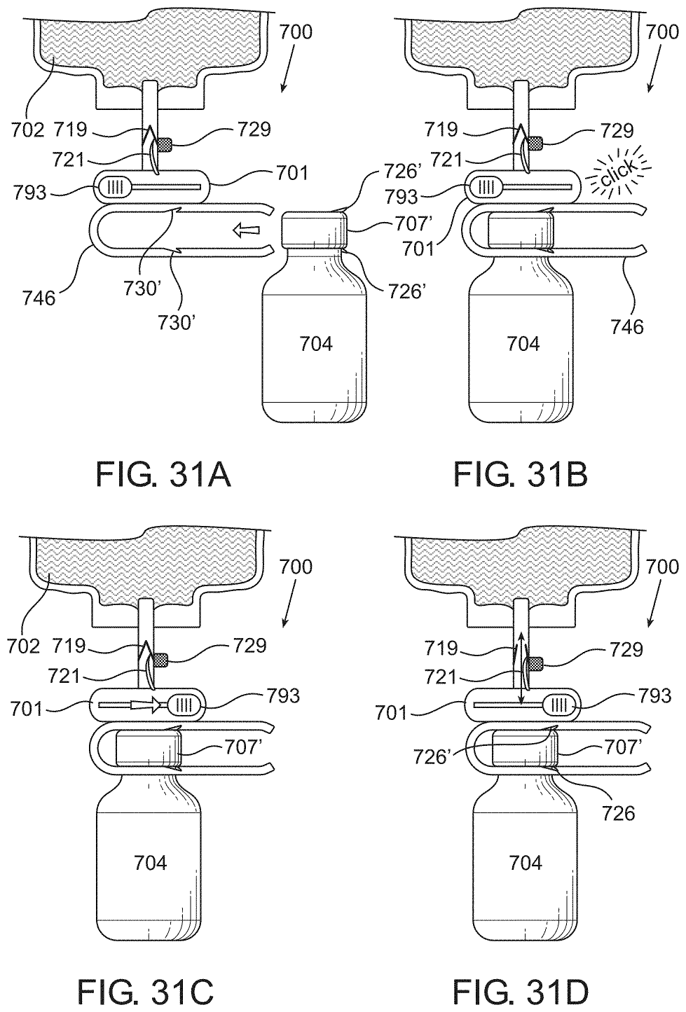

FIGS. 31A-31D present system 700 which includes an engagement mechanism allowing engagement between vessel 704 and housing 701 via clamping rail 746. A port of vessel 704 can be engaged within clamping rail 746 and slide there along until reaching a longitudinal alignment with vessel 702. A fluid passageway can be formed between the vessels following opening of valve 719 by valve unlocking mechanism 721 and valve actuator 729. FIG. 31A shows vessel 704 and housing 701 when spaced apart and AA surround the vessel 704. In FIG. 31B, vessel 704 slides along the clamping rail 746 until reaching a longitudinal alignment with vessel 702. The wiping member is then actuated by a user using wiping member actuator in the form of tab 793 (FIG. 31C). A fluid passageway can be formed upon opening of valve 719 (FIG. 31D). The locking mechanism here include dents 730′ disponed on clamping rail 746, at opposing sides thereof and teeth 726′ protruding from a top and a bottom surface of port 707′. The teeth 726′ are configured to couple/engage with dents 730′. The coupling/engagement of teeth 726′ and dents 730′ may be a permanent and/or irreversible engagement. The teeth 726′ may be made of a material of sufficient strength to prevent detachment of vessel 704 from dents 730′. The material may be a metal, a metal alloy, a rigid plastic, a semi-rigid plastic, and combinations thereof.

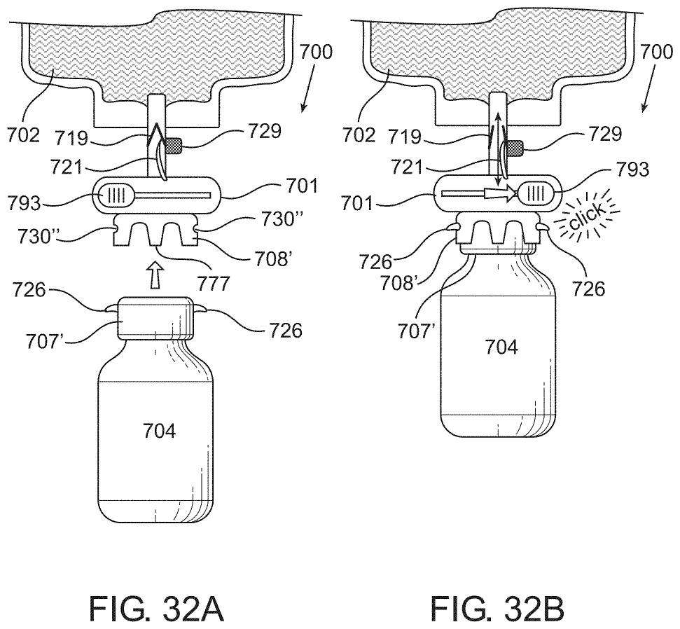

FIGS. 32A-32B demonstrate system 700 which includes an engagement mechanism in the form of ratchet teeth 777 disposed at the bottom wall of housing 701. The locking mechanism includes teeth 726 and dents/holes 730″. Optionally, ratchet teeth 777 may be disposed directly on vessel 702 (for example ratchet teeth 777 having dent/hole 730″ may abut a wall of vessel 702, may be flush mounted to vessel 702, and/or may be surface mounted to vessel 702. As vessel 704 is moved upwards towards the ratchet teeth 777 teeth 726 may lie against side wall of port 707′ and/or may retract into port 707 to allow engagement/coupling of vessel port 707′ and ratchet teeth 777. When teeth 726 align with dent/hole 730″ teeth 726 may then extend into and/or through hole 730″ thus providing a permanent/irreversible engagement between port 707′ and ratchet teeth 730″. Vessel 702 and vessel 704 are shown as bag and vial respectively. However, the bag and the vial are used arbitrarily, and any vessel/device may replace vessel 702 and/or vessel 704 such as (but not limited to) a syringe, a connector, a filter, a buretrol, a manifold, a container, a container with a plunger, a container with a moveable wall, a container with an expulsion member, an infusion line, etc.

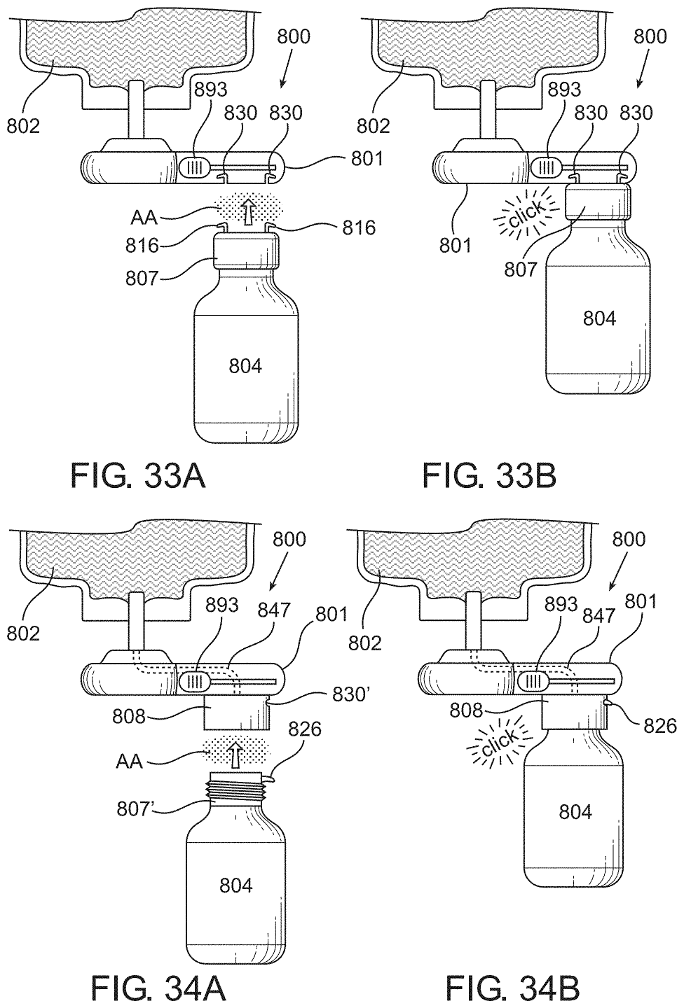

FIGS. 33A-33B demonstrate yet a further exemplary system 800 which combines movement of the vessel 804 and a movement of a wiping member within housing 801 of the decontamination device. The steps of decontamination in system 800 include a connection of vessel 804 to housing 801 via a locking mechanism of hooks 816 and dents 830. Hooks 816 disposed to extend and upwardly protrude from the surface of vessel 804. Dents 830 disposed in the lower surface of a first compartment of housing 801. Following attachment of vessel 804 to housing 801 (FIG. 33B), the vessel 804 can be wiped by a wiping member through tab 893 and then moveable to align longitudinally with vessel 802 (not shown). Upon alignment of the vessel a fluid passageway may be formed (not shown). In FIGS. 34A-34B, system 800 is shown when further comprising internally threaded port 808 (not shown) which can engage with threaded port 807′. In this alternative embodiment of system 800, the vessel 804, once attached is stationary and disposed in a longitudinal axis being parallel to the longitudinal axis of vessel 802. A fluid passageway is formed by virtue of channel 847 disposed in the interior of housing 801. Vessel 804 is shown having a threaded port 807′ and a tooth 826. The tooth 826 may be a retractable tooth. The tooth 826 may be disposed on threads 807′, between threads 807′, above the threads 807′ (as shown), etc.

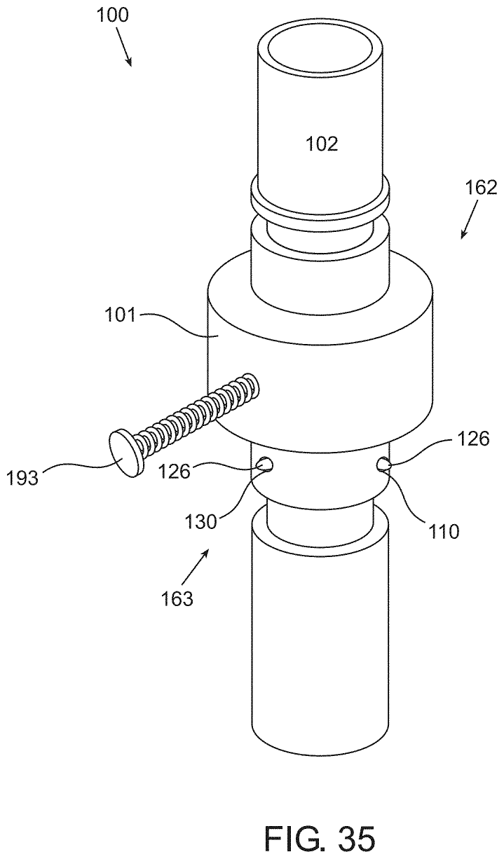

Referring to FIG. 35, a perspective view of yet a further exemplary system 100 is shown. System 100 includes a housing 101 with top port 162 allowing engagement with vessel 102 and bottom port 163 allowing engagement with vessel 104. A wiping member is disposed within the housing (not shown). The system includes a wiping member actuator in the form of handle 193. The system further includes teeth 126 and dents 130/110 for allowing a permanent lock between vessels and a decontamination device. The teeth 126 may be retractable teeth.

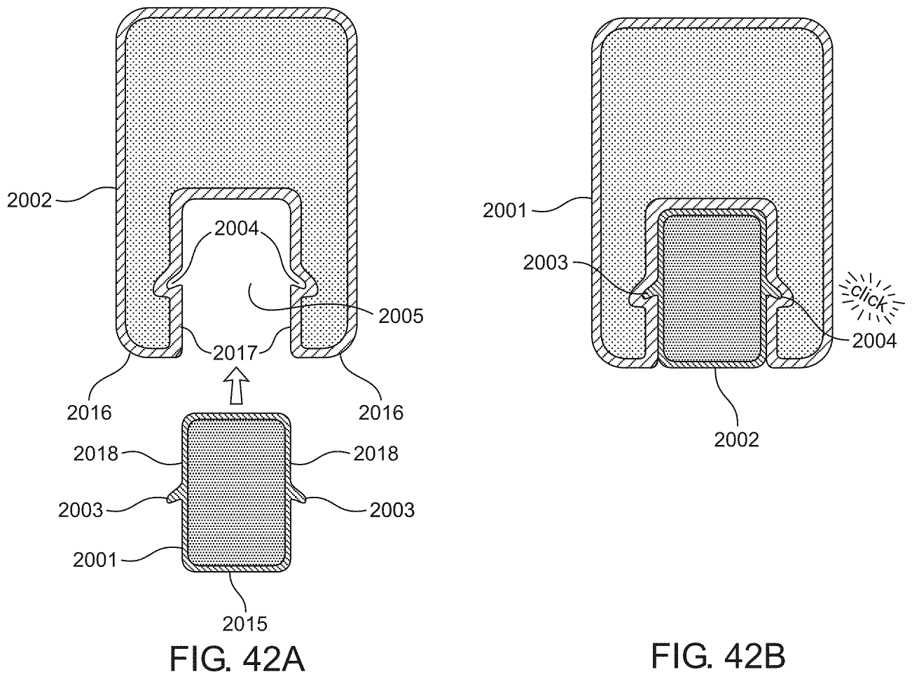

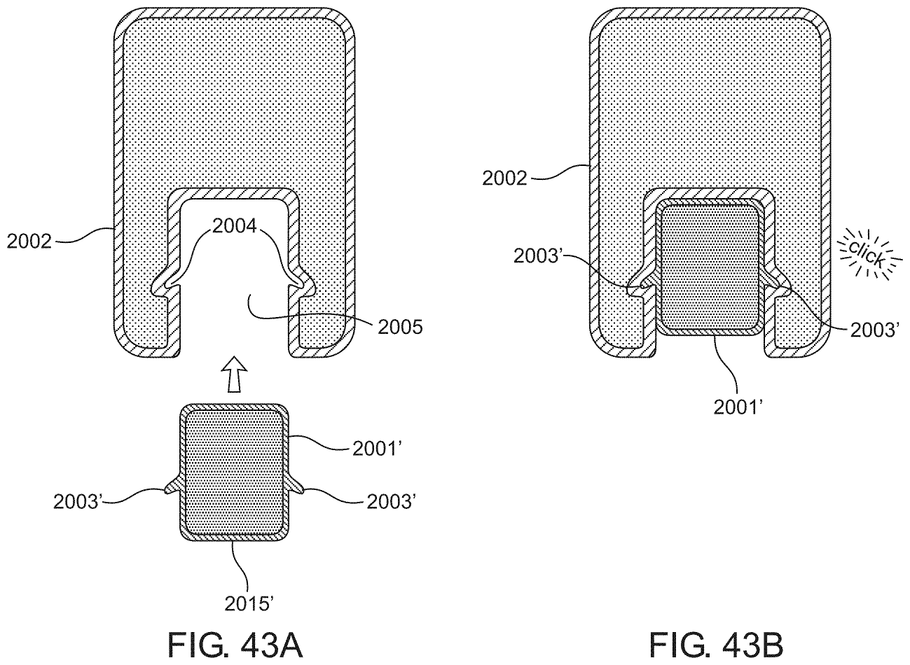

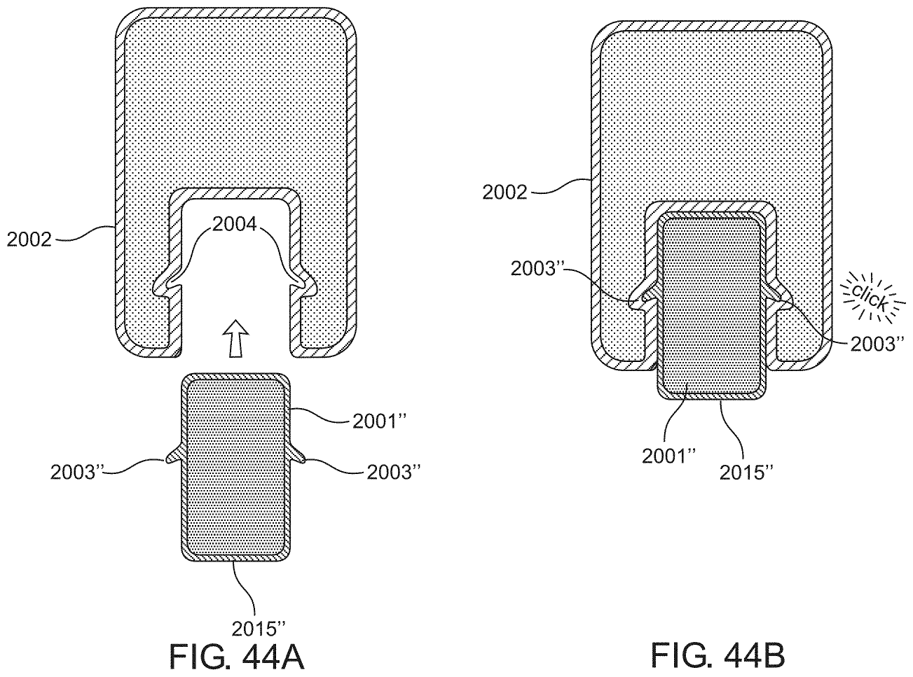

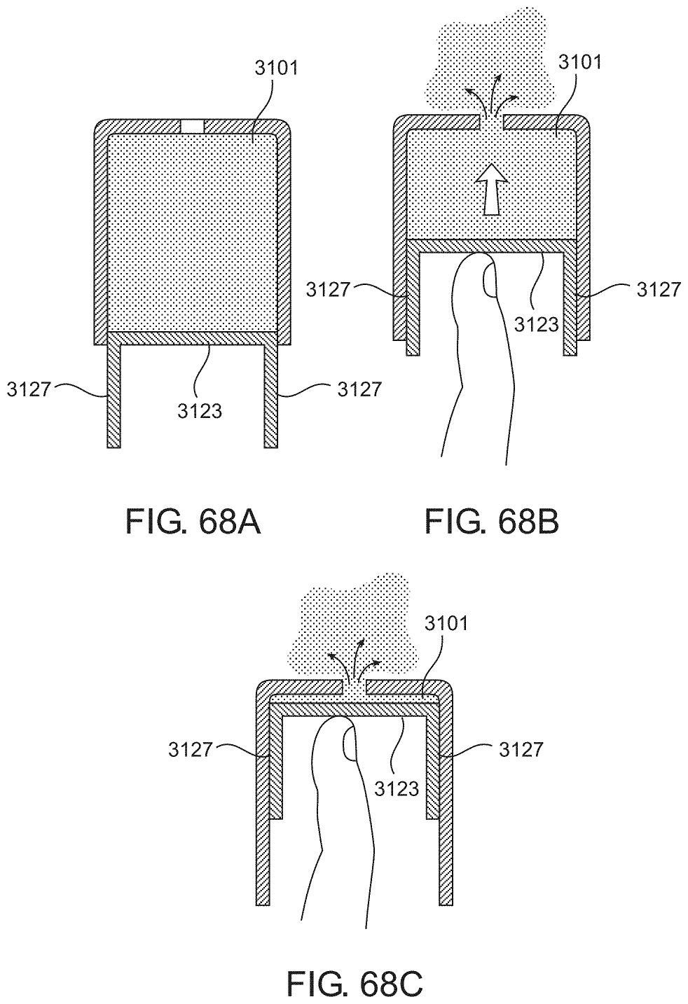

FIGS. 42A-77C illustrate various cartridges that can be coupled to various types of vessels (e.g., a bag). The cartridges configured to contain a medical substance and to fill the vessel with the substance. The cartridges may be made of various materials and various combinations of materials. In one or more embodiments, the cartridges may be made of a glass material. In one or more embodiments, the cartridges may be made of a metal and/or a metal alloy. In one or more embodiments, the cartridges may be made of a plastic material. The plastic may be a rigid plastic. In one or more embodiments, the plastic may be a semi-rigid plastic. In one or more embodiments, the cartridges may be made of at least two materials, the first material being a glass material disposed on the interior of the cartridge (wherein the glass comes in contact with a medical substance disposed inside the cartridge) and the second material being a plastic material disposed on the exterior of the cartridge (so as to prevent cracking/breakage of the cartridge during shipping/falling to the floor). The plastic may be a rigid plastic in some embodiments. In some embodiments, the plastic disposed on the exterior of the cartridge may be a semi-rigid plastic. In one or more embodiments, the interior of the cartridge may be made of a glass material and the exterior of the cartridge may be made of a rubber material. As illustrated in the following FIGS. 42A-44B, vessel 2002 can include a cavity 2005 into which a cartridge 2001 may be loaded (disposed). A locking mechanism in the form of teeth 2003 may be disposed on an outer surface of walls 2018 of cartridge 2001 and may project outwardly therefrom. The teeth 2003 may be locked into dents 2004 in a wall 2017 of cavity 2005. An audible sound such as a “click” sound or any other sound may be emitted at about the time, or at the time of engagement or locking of cartridge 2001 to vessel 2002. Teeth 2003 may be retractable teeth operable between a retracted position (not shown) and an extended position (shown). Various cartridge types with various sizes and shapes are contemplated. In an exemplary embodiment, a bottom wall 2015 of a cartridge 2001 may align the bottom wall 2016 of vessel 2002 (FIGS. 42A-42B). Alternatively, cartridge 2001′ may be sized and shaped such that none of the walls protrudes from the vessel 2002. In accordance with this embodiment, a bottom wall 2015′ of cartridge 2001′ is located entirely inside cavity 2005 (FIGS. 43A- 43B). According to this embodiment, after the cartridge 2001′ is disposed/loaded inside cavity 2005 of vessel 2002 a user is unable to hold/grasp cartridge 2001′ to pull out cartridge 2001′ from the cavity 2005. This is very beneficial as it prevents detachment of cartridge 2001′ from vessel 2002. Alternatively, the cartridge 2001″ may be sized and shaped such that the bottom wall 2015″ protrudes from a bottom wall 2016 of vessel 2002 (FIGS. 44A-44B). Vessel 2002 may be a container such as a bag or a bottle. Vessel 2002 may have a rigid wall, a flexible wall, and combinations thereof. Vessel 2002 may also have fortified walls that maintain cavity 2005's shape and/or size to allow cavity 2005 to accept cartridge 2001. The fortified wall may be made of a rigid plastic, glass, metal alloy, and combinations thereof. Vessel 2002 may be any type of container/device that will accept cartridge 2001.

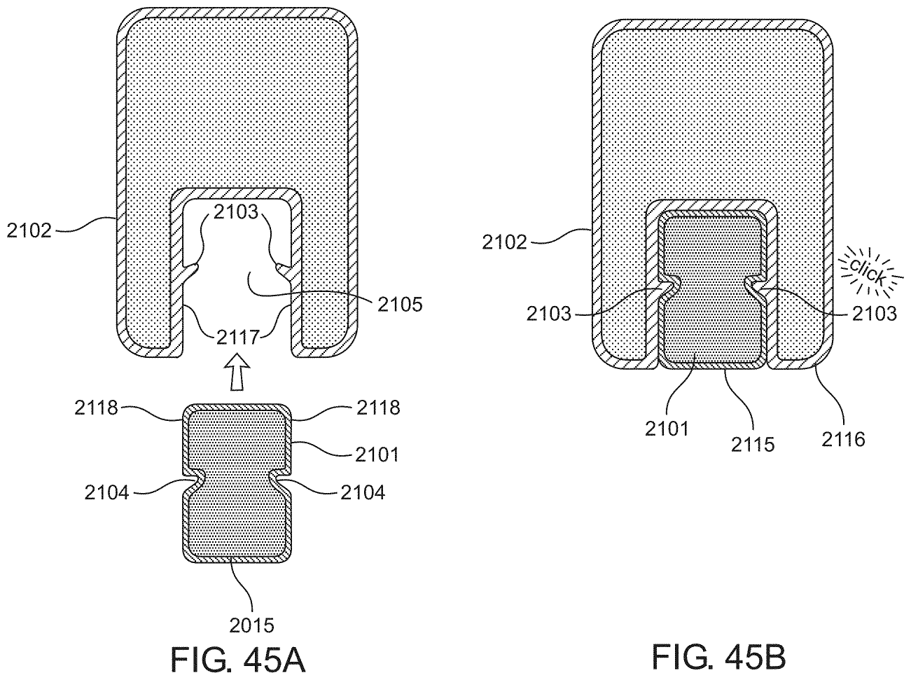

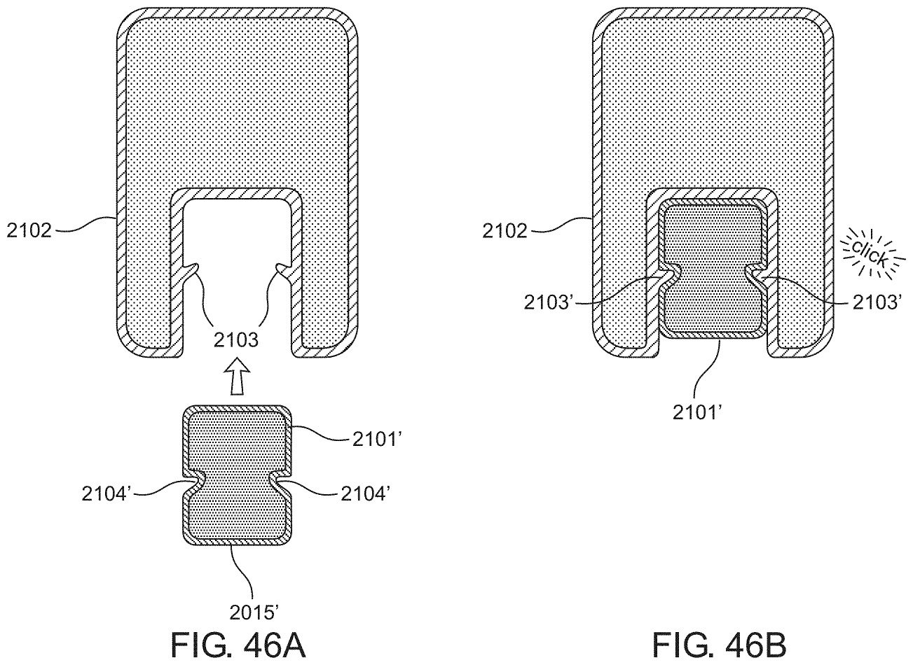

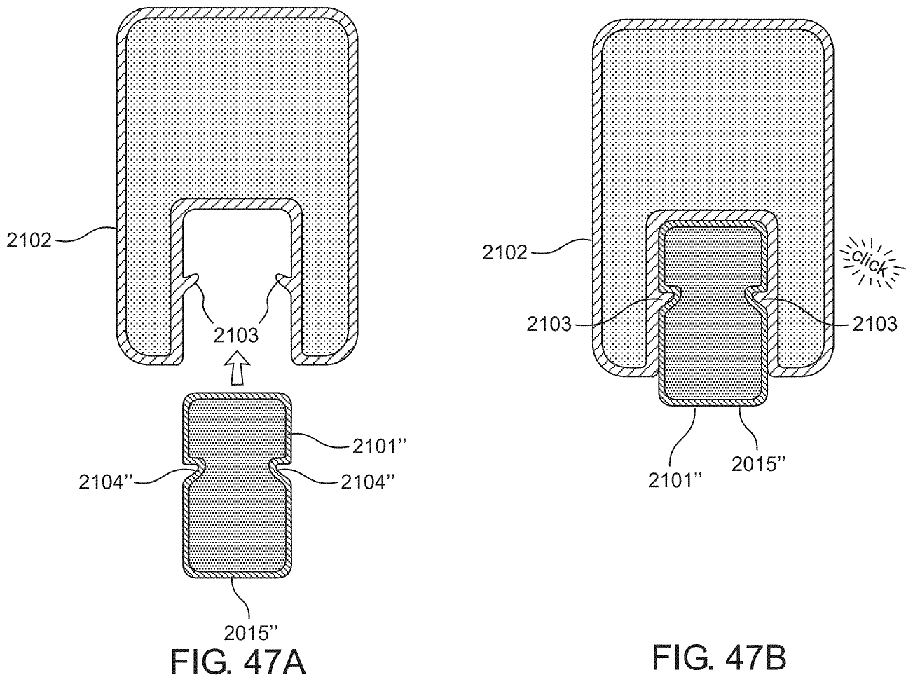

In alternative embodiments, the locking mechanism includes locking members in the form of teeth 2103 protruding from an outer surface of side walls 2117 of cavity 2105 and securing members in the form of dents 2104 disposed in side walls 2118 of cartridge 2101. A bottom wall 2115 of the cartridge 2101 may align with the bottom wall 2116 of vessel 2102 (FIGS. 45A-45B) when cartridge 2101 is loaded/disposed in cavity 2105 of vessel 2102. Alternatively, the cartridge 2101′ may be inserted or loaded into vessel 2102 such that a bottom wall thereof 2115′ is located entirely inside cavity 2105 (FIGS. 46A-46B) thus preventing a user from being able to hold/grasp cartridge 2101′ and detach/pull cartridge 2101′ from vessel 2102. Alternatively, the cartridge 2101″ may be inserted or loaded into cavity 2105 such that the bottom wall thereof 2015″ protrudes from a bottom wall of vessel 2102 (FIGS. 47A-47B). Teeth 2103 may be, in one or more embodiments, retractable teeth.

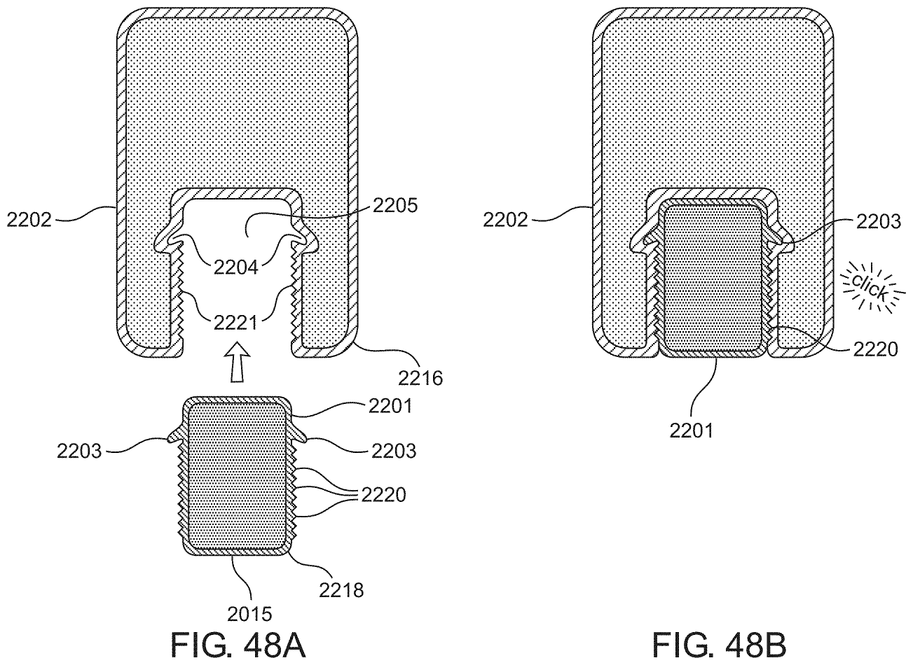

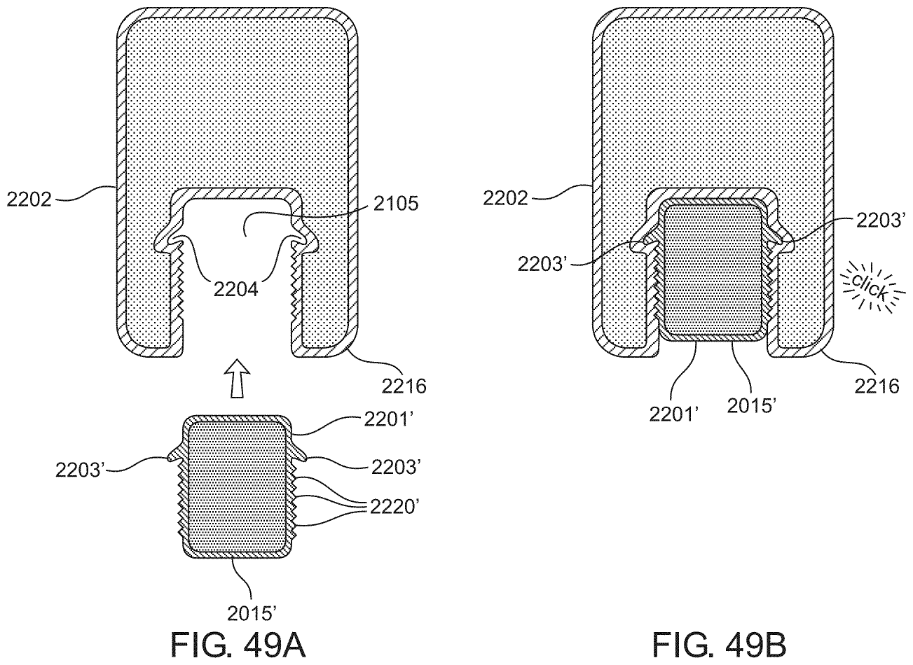

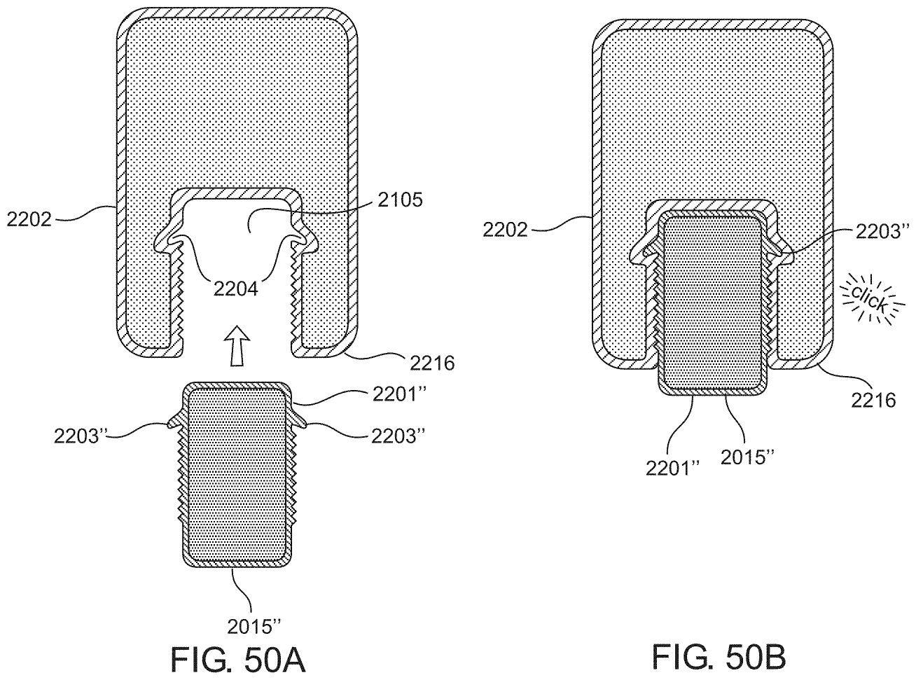

As shown in FIGS. 48A-50B, a cartridge 2201 may be externally threaded such to include threads 2220 on side walls 2218. Threads 2220 are configured to cooperate with internal threads 2221 of cavity 2205. A bottom wall 2015 of the cartridge 2201 may align with the bottom wall 2216 of vessel 2002 (FIGS. 48A-48B). It is important to note that threads 2220 are disposed along a length of cartridge 2201. In one or more embodiments, threads 2220 are disposed along a length (or width) of a body of cartridge 2201. This is an important feature since it provides a fixed/sturdy engagement between cartridge 2201 and internal threads 2221 of vessel 2202. If a toxic substance, such as a chemotherapeutic substance, is disposed inside cartridge 2201 a fixed/sturdy engagement between cartridge 2201 and vessel 2202 is of extreme importance as this may reduce and/or prevent detachment, optionally spontaneous detachment, of cartridge 2201 from vessel 2202. Alternatively, the cartridge 2201′ may be inserted or loaded into vessel 2202 such that a bottom wall thereof 2015′ is located entirely inside cavity 2105 (FIGS. 49A-49B) thus preventing a user from grasping and detaching cartridge 2201′ from vessel 2202. Alternatively, the cartridge 2201″ may be inserted or loaded into cavity 2105 such that the bottom wall thereof 2015″ protrudes from a bottom wall 2216 of vessel 2202 (FIGS. 50A-50B). In one or more embodiments, the thread disposed on the body of cartridge 2203″ may be disposed along the entire length of the cartridge body. In one or more embodiments, the thread disposed on the body of cartridge 2203″ may be disposed along a partial length of the cartridge body. In one or more embodiments, the thread may be made of a plastic and/or a rubber substance. In one or more embodiments, the thread may be made of an elastomeric material. In one or more embodiments, the teeth 2203″ of FIGS. 50A-50B may be retractable teeth. In one or more embodiments, the bottom wall 2015″ of cartridge 2201″ of FIGS. 50A-50B may be a moveable wall (not shown). In one or more embodiments, the moveable wall may be a plunger. In one or more embodiments, the moveable wall may be made of an elastomeric material. In one or more embodiments, the moveable wall may be made of a rubber material.

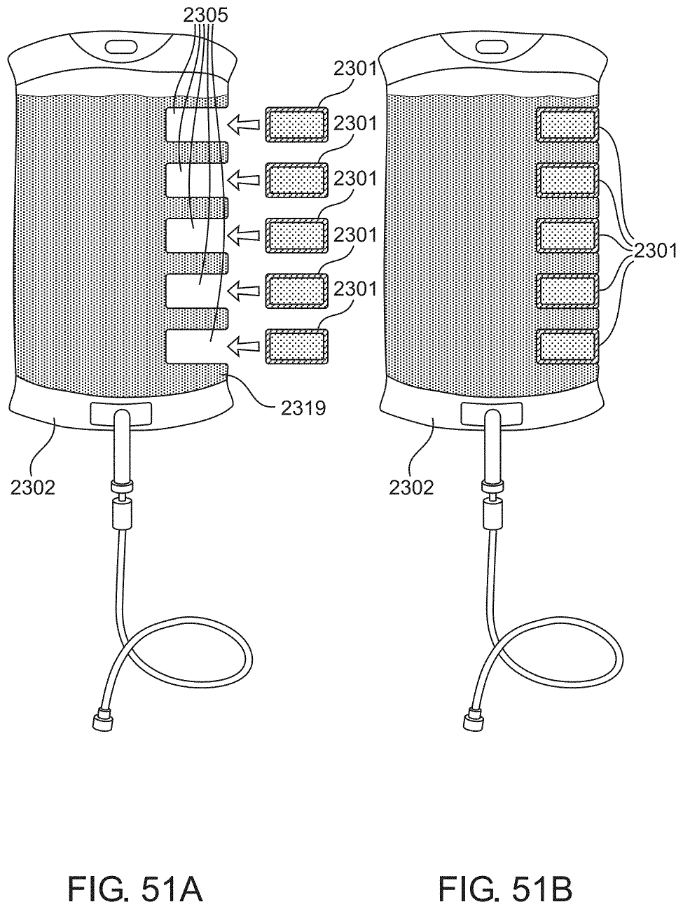

The following FIGS. 51A-56 illustrate a vessel, such as an infusion bag that can accommodate a plurality of cartridges. In one embodiment, the vessel is configured to accommodate one cartridge. In one embodiment, the vessel is configured to accommodate a plurality of cartridges. In one embodiment, the vessel is configured to accommodate two or more, or three or more, or four or more or five or more cartridges. In one embodiment, the vessel includes one cavity for accommodating one cartridge. In one embodiment, the vessel includes a plurality of cavities for accommodating a plurality of cartridges. In one embodiment, the vessel includes two or more, or three or more, or four or more, or five or more cavities for accommodating a respective number of cartridges.

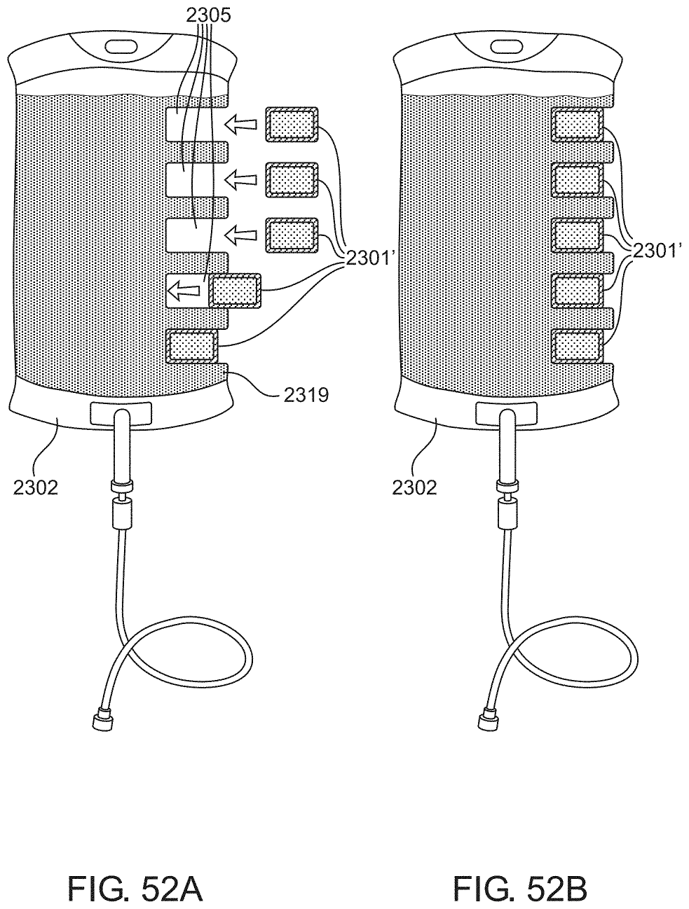

FIGS. 51A-52B illustrate a vessel, here bag 2302 which includes five cavities 2305 for accommodating five cartridges 2301/2301′. The side/bottom wall of the cartridges 2301 may align a side wall 2319 of vessel 2302 (FIGS. 51A-51B). Alternatively, a side wall of cartridge 2301′ may be located entirely within cavity 2305 (FIGS. 52A-52B), such that none of the walls of cartridge 2301′ protrudes from the walls 2319 of vessel 2302 thus preventing a user from grasping/grabbing/holding and detaching cartridges 2301/2301′ from bag 2302.

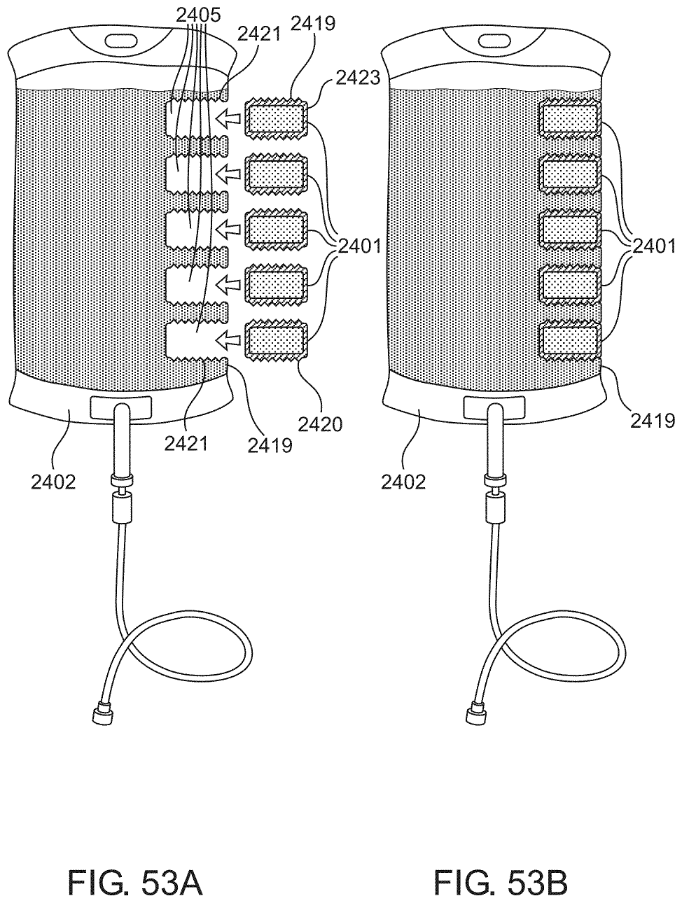

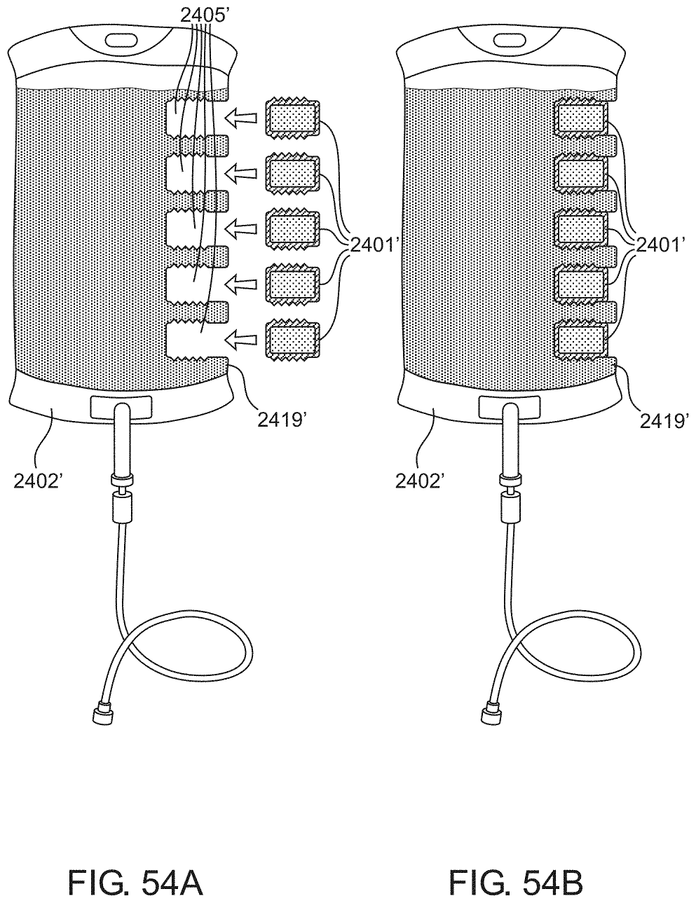

FIGS. 53A-54B illustrate a vessel, here bag 2402 which includes multiple cavities 2405 with internally threaded walls 2421, suitable for accommodating cartridges 2401 with externally threaded side walls 2420. The side wall 2423 of the cartridges 2401 may align a side wall 2419 of vessel 2402 (FIGS. 53A-53B). Alternatively, the walls of cartridges 2401 may be located entirely inside cavities 2405′ (FIGS. 54A-54B).

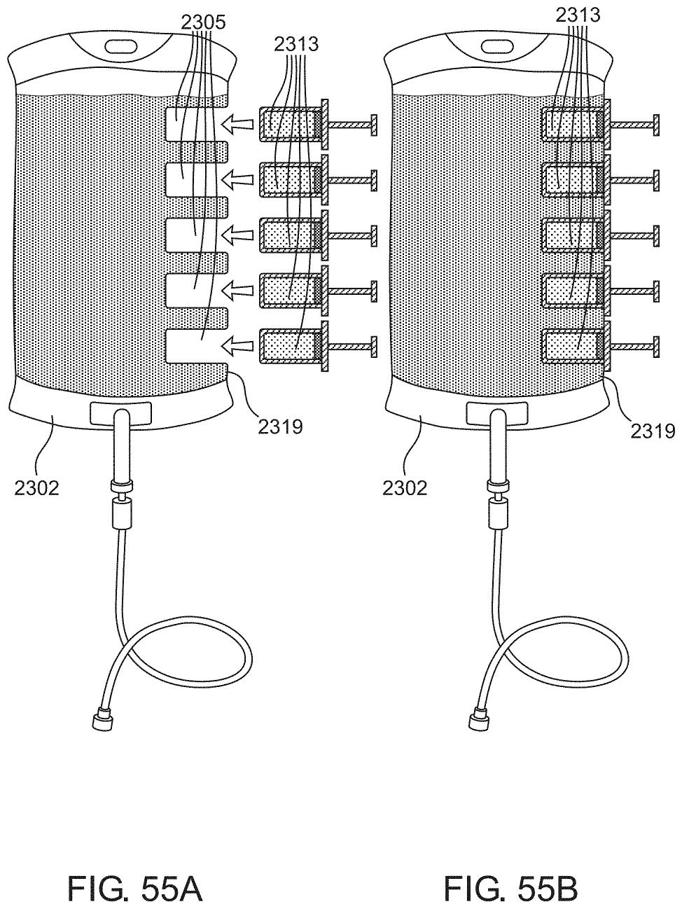

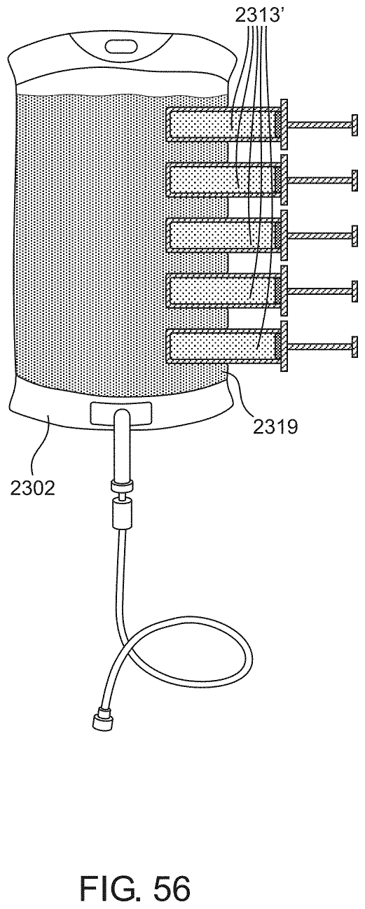

In FIGS. 55A-56, vessel 2302 is shown when accommodating cartridges 2313 having a form of a syringes. Here five syringes 2313 are shown, but alternative number of syringes 2313 is further contemplated, such as two or more, three or more, etc. The side wall of syringes 2313 (not including the piston part) may align a side wall 2319 of vessel 2302 (FIGS. 55A-55B). Alternatively, the walls of syringes/cartridges 2313′ may extend outside the cavities 2305 (FIG. 56).

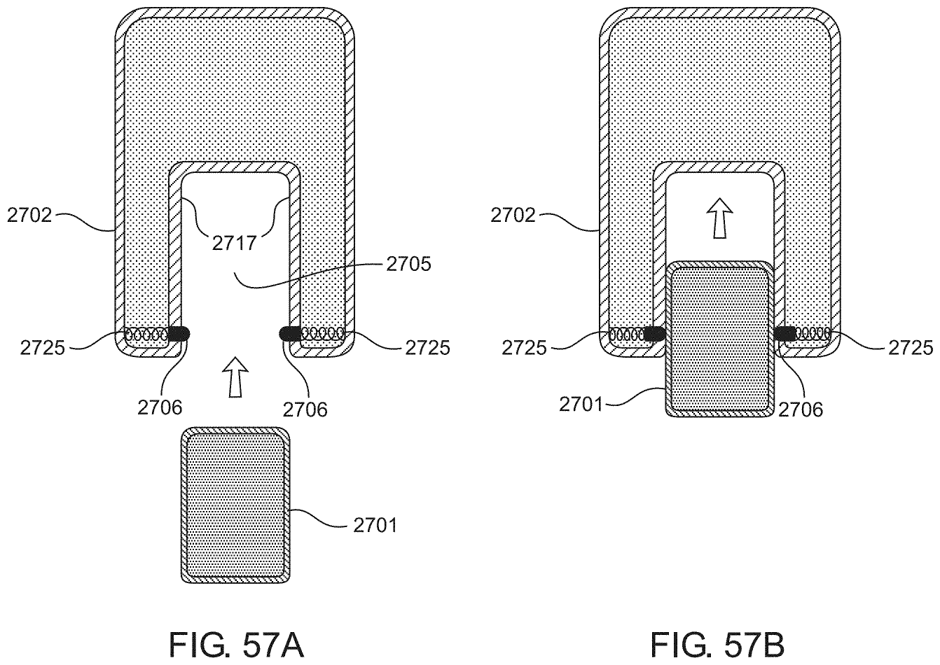

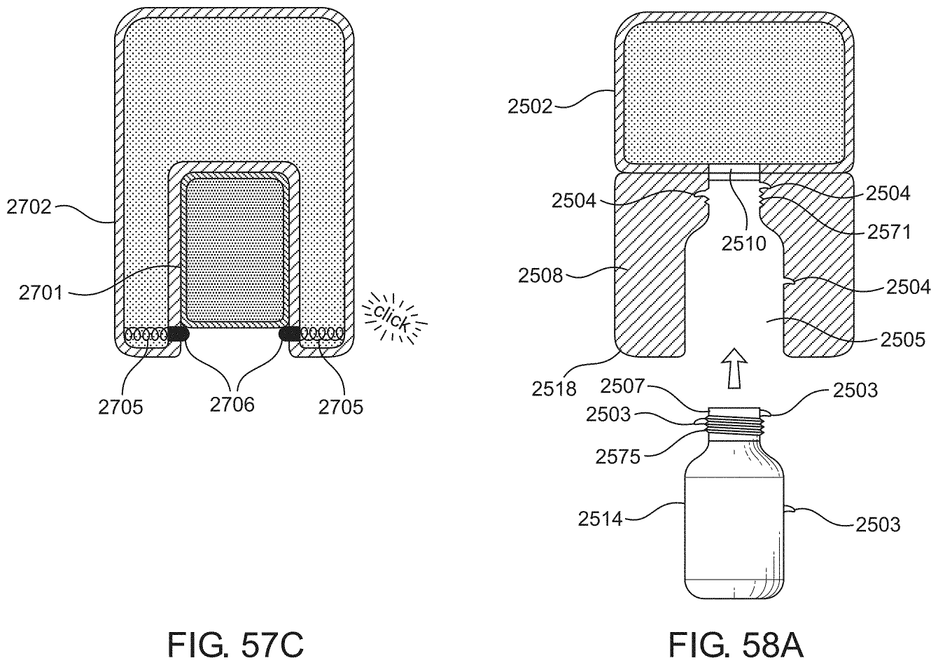

FIGS. 57A-57C illustrate another exemplary locking mechanism of vessel 2702. Vessel 2702 includes pins 2706 on opposing sides of the external surface of side walls 2717 of cavity 2705. Side walls 2717 may be made of a rigid material that maintains the shape/contour of cavity 2705 such as (but not limited to) a metal alloy, a rigid plastic, a semi-rigid plastic, a rubber material, a glass material, and combinations thereof. Pins 2706 can be spring loaded or attached to spring 2725. The pins 2706 are retractable from a retracted position to a fully extended position. When the cartridge 2701 is inserted by a user into cavity 2705, pins 2706 retract by the pushing forces applied thereon, allowing insertion of cartridge 2701 within the cavity 2705 (FIG. 57B). After full positioning of the cartridge 2701 within cavity 2705, the pins 2706 can extend to lock or prevent any movement or release of the cartridge from the cavity 2705. The pins 2706 may be made of a plastic and/or a rubber material.

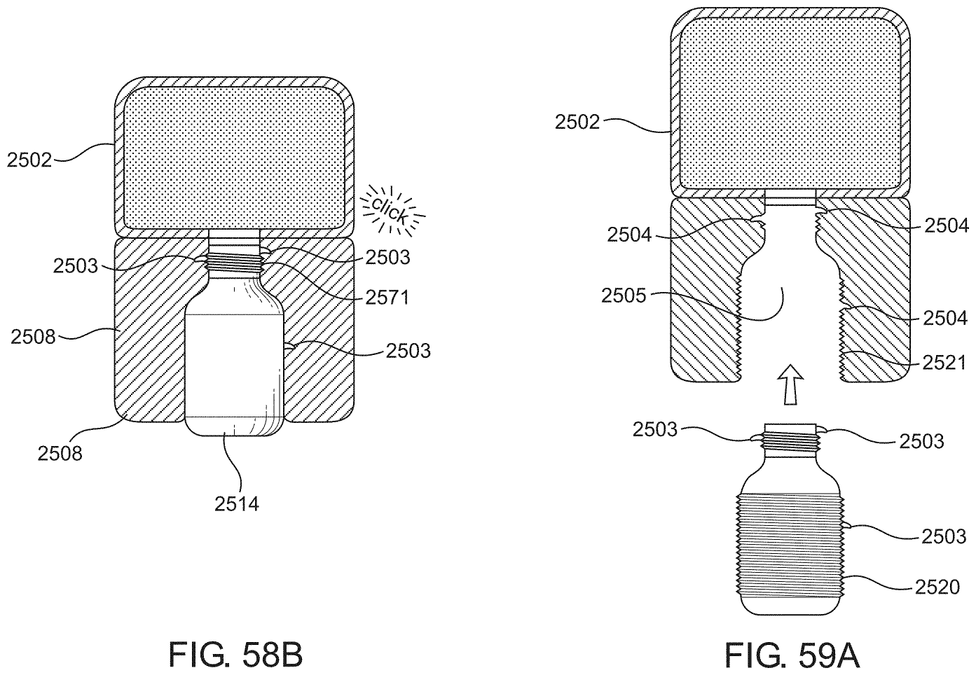

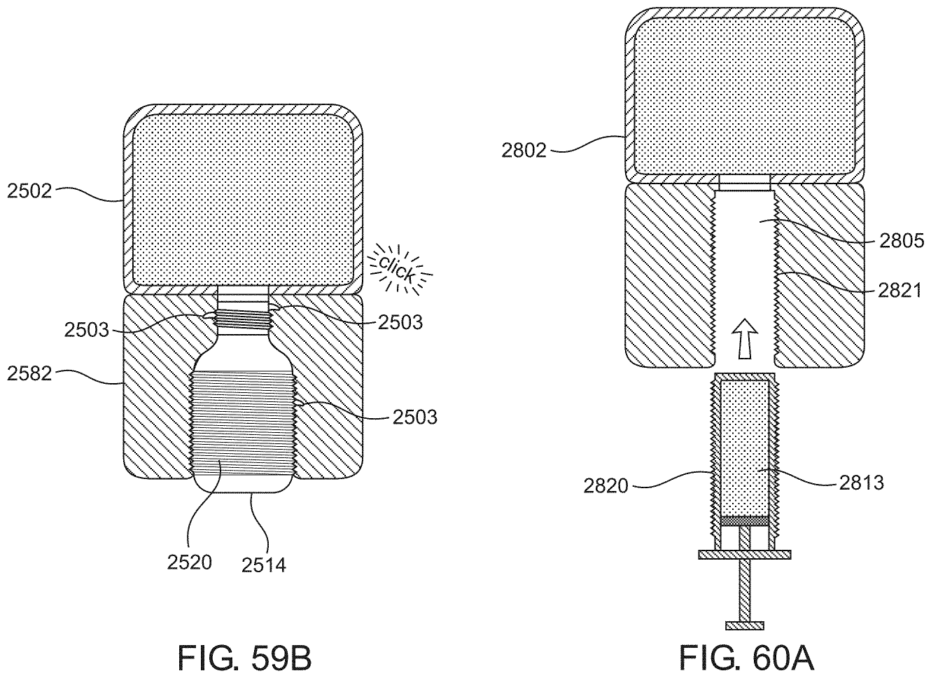

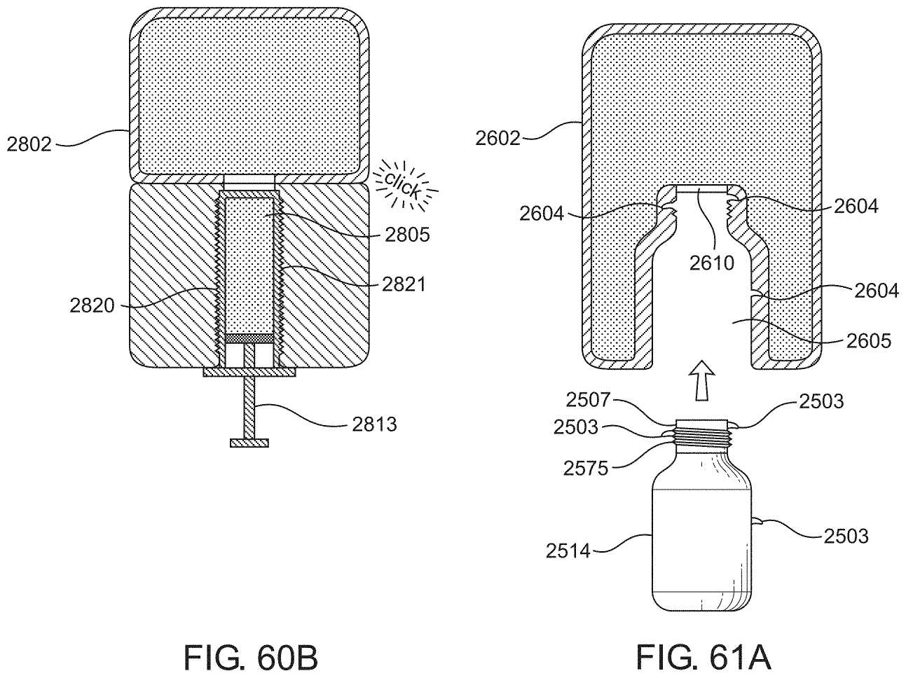

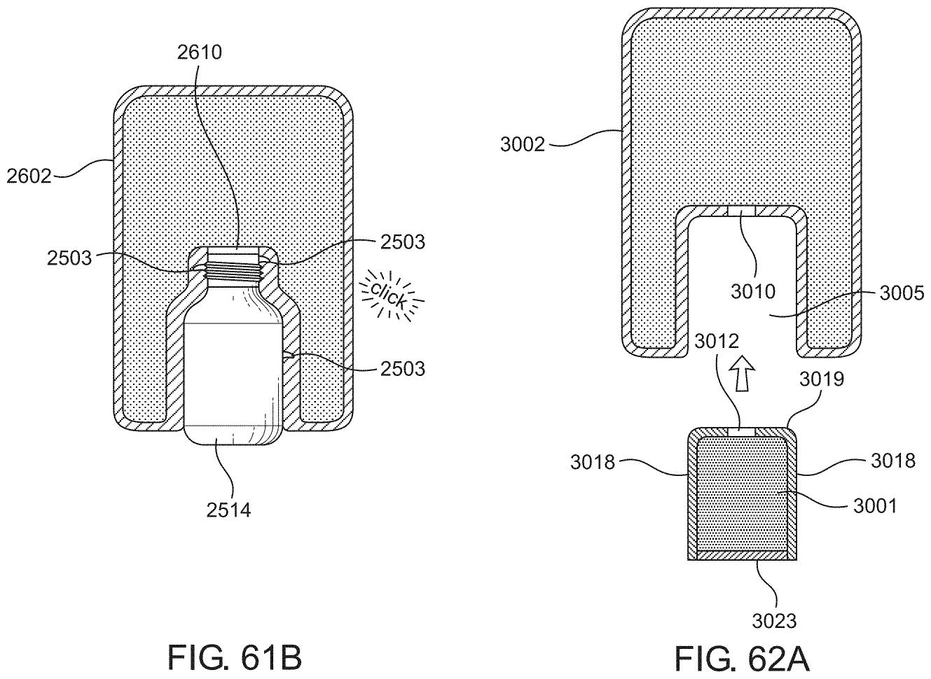

FIGS. 58A-58B illustrate a vessel 2502 configured to accommodate cartridge in the form of vial 2514. Here the vessel 2502 includes a port 2508 with vertical wall surrounding a vessels' opening 2510 and enclosing a cavity 2505 configured to accommodate vessel 2514. The port 2508 in this case includes a cavity 2505 having a cavity like form for accommodating a cartridge having a form of a vial 2514. The port 2508 includes one or more internal dents 2504 for locking inside teeth 2503 disposed on side walls of vial 2514. Teeth 2503 may be retractable teeth operable between a retractable position (not shown) and an extended position (shown). Vial 2514 may be threaded into port 2508 via threads 2575 and 2571. A click sound may be emitted (FIG. 58B) at about the time of engagement/locking of the vessel into port 2508. Optionally, internal walls of cavity 2505 include internal threads 2521 which can cooperate with threads 2520 disposed externally surrounding vessel 2514 (FIG. 59A-59B). FIG. 60A-60B shows vessel 2802 having a cavity 2805 defined by cavity side walls having a thread 2821 configured to engage/couple with thread 2820 of syringe 2813. Syringe 2813 having a thread 2820 disposed along the exterior surface of the barrel of syringe 2813. Syringe 2813 may have a beneficial substance such as a medicament disposed within the syringe and vessel 2802 may have a beneficial substance such as a diluent and/or a medicament disposed within vessel 2802. In an alternative embodiment, the cavity 2605 is made an integral part of vessel 2602 (FIG. 61A-61B). According to this embodiment the cavity 2605 walls are made of a rigid or a semi-rigid material to maintain the shape/contour of the cavity 2605. The cavity walls may be made of a rigid plastic, a rubber, a glass material, a metal alloy, and combinations thereof. Vessel 2602 may be a container, a bag, a bottle, etc. Optionally, or alternately, vessel 2602 may be entirely made of a rigid wall, a flexible wall, and combinations thereof. Vessel 2602 may be made of a plastic material, a glass material, an elastomeric material, and combinations thereof.

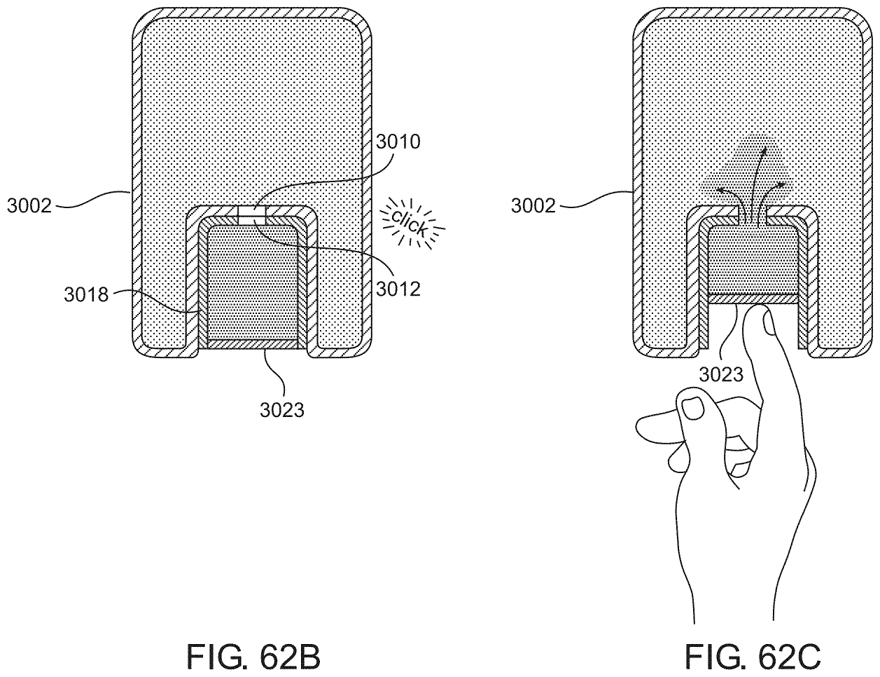

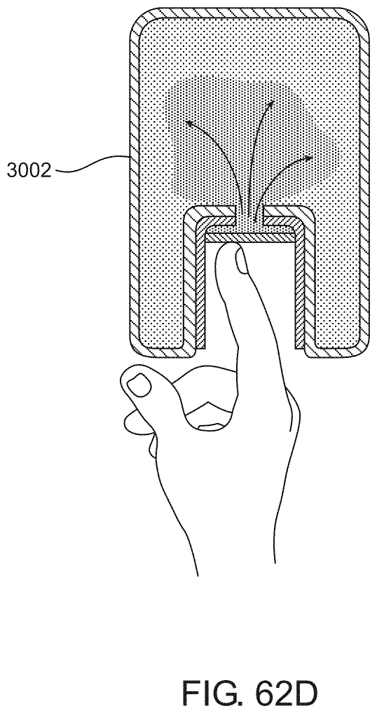

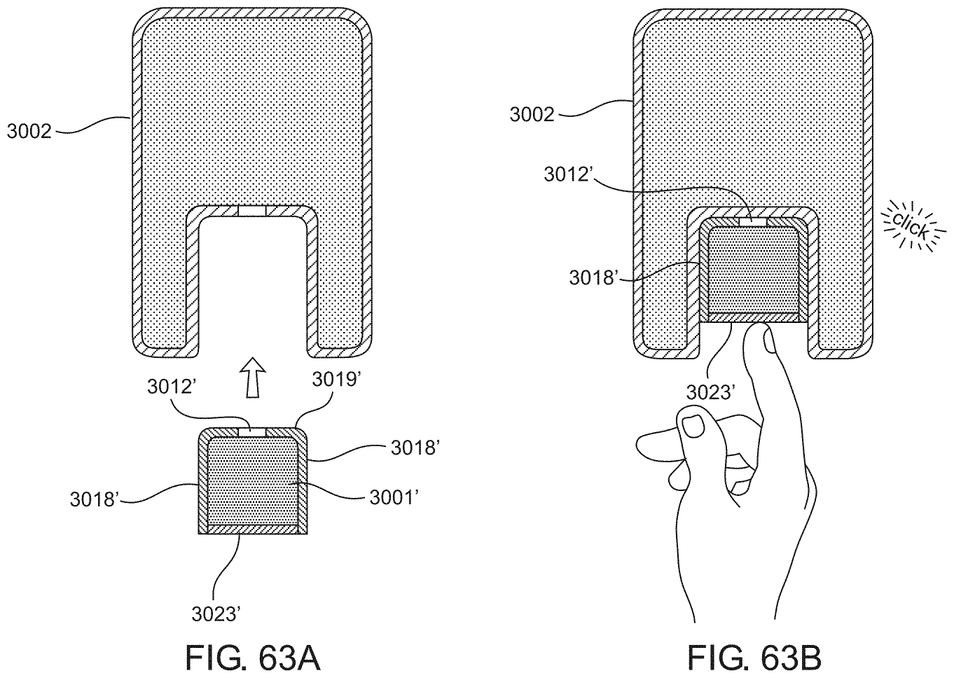

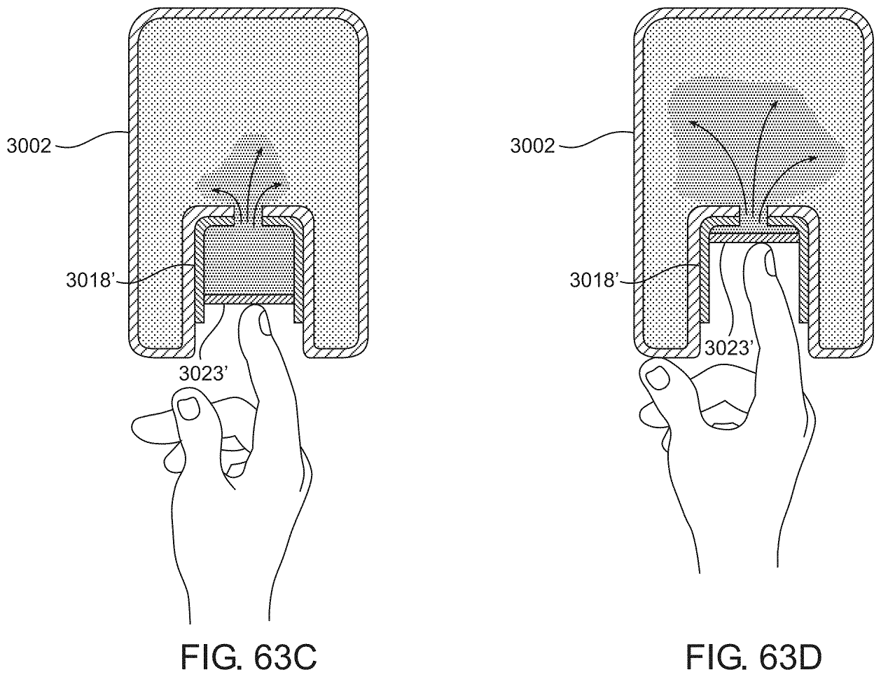

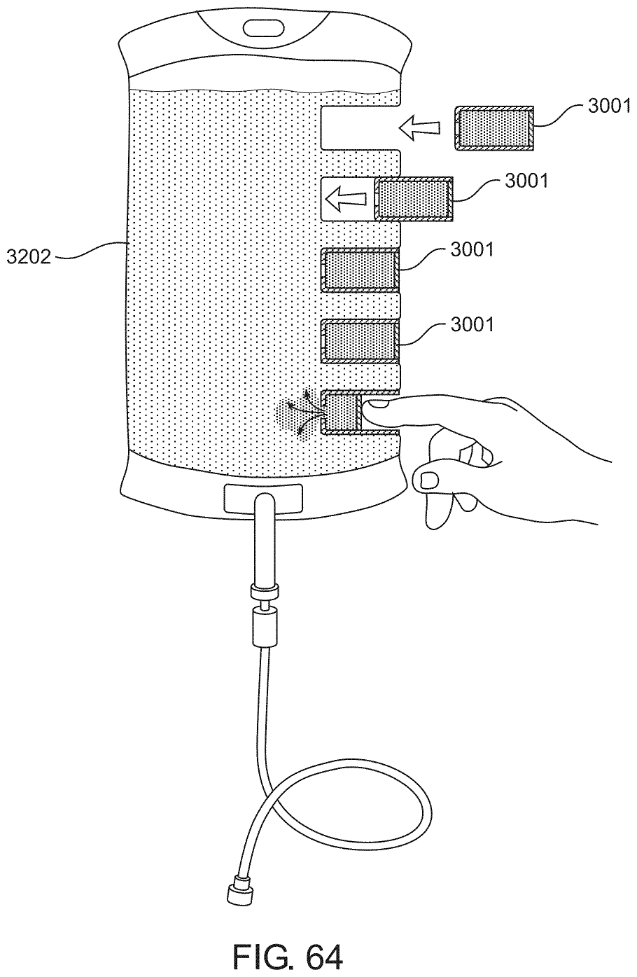

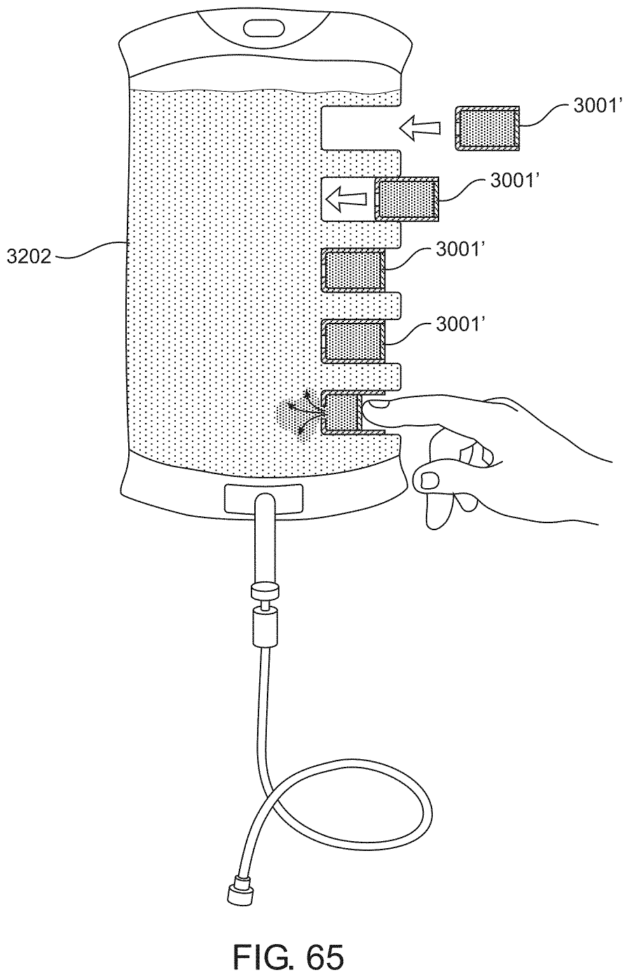

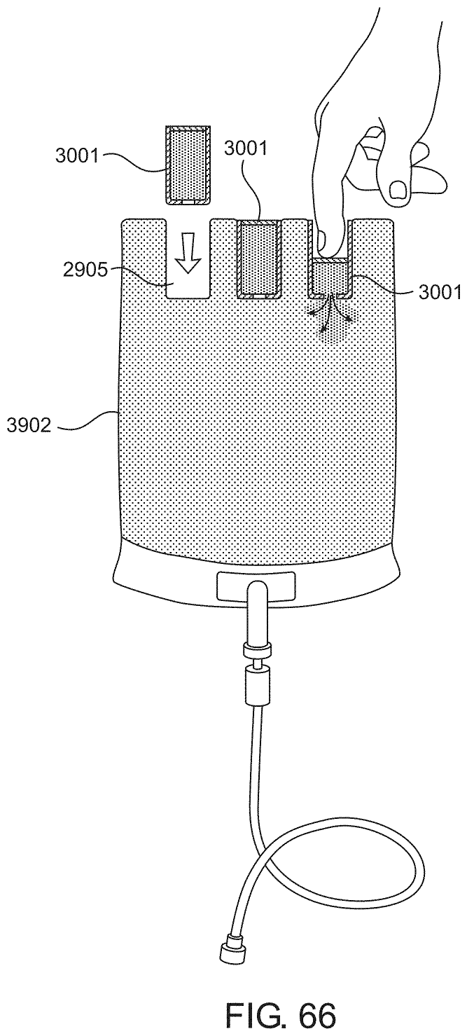

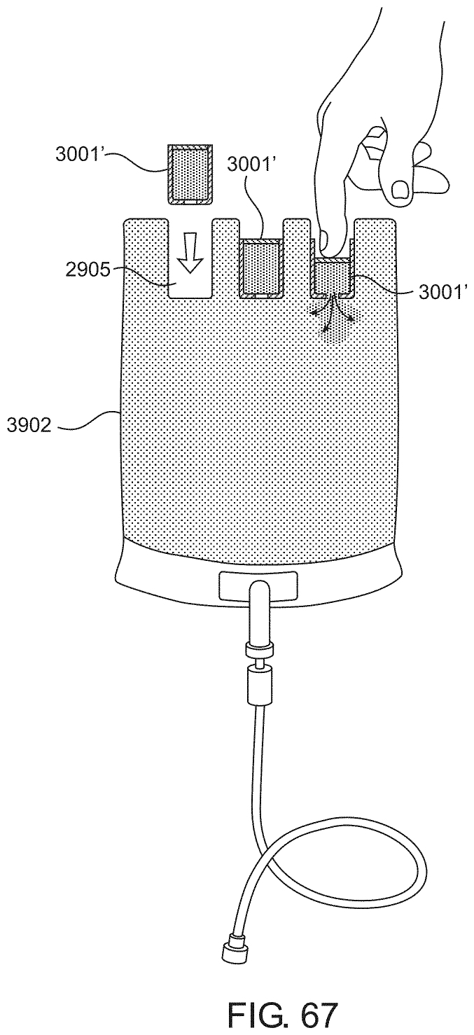

The following FIGS. 62A-77C illustrate possible fluid transfer mechanisms for transferring fluid from a cartridge to a vessel. FIGS. 62A-62D schematically illustrate a cartridge 3001 with side walls 3018, an upper wall 3019 and a plunger 3023. An opening 3012 for transferring fluid (such as a medical substance) extends in top wall 3019. The cartridge 3001 is configured to enter and reside in cavity 3005 of vessel 3002 which also has a vessel's opening 3010 for receiving fluid from the cartridge 3001. The fluid transfer mechanism includes a moveable plunger or plunger structure 3023 attached to wall 3018. The user can push plunger 3023 towards the vessel's opening 3010, allowing for a fluid transfer from the cartridge 3001 to the vessel 3002. When pushed towards opening 3012, moveable plunger 3023 slides along walls 3018, optionally, via a sliding mechanism (not shown). The sliding mechanism may be a rail mechanism disposed along an interior wall of side cartridge 3018. It is important to note that cartridge 3001 does not have a handle thus preventing a user from pulling and detaching the moveable plunger 3023 from the cartridge 3001. The plunger 3023 can only be pushed towards opening 3012 using a finger of a user (the plunger 3023 cannot be pulled and detached from the cartridge 3001). In FIGS. 63A-63D a shorter cartridge 3001′ is shown which when resides within vessel 3002 does not protrude from the walls of vessel 3002. The cartridges 3001 and/or 3001′ can be used to feed vessel 3202, or vessel 3902 which includes a plurality of cavities disposed on at least one side wall of vessel 3202 (FIGS. 64-65) or a plurality of cavities 2905 disposed at a top wall thereof (FIGS. 66-67).

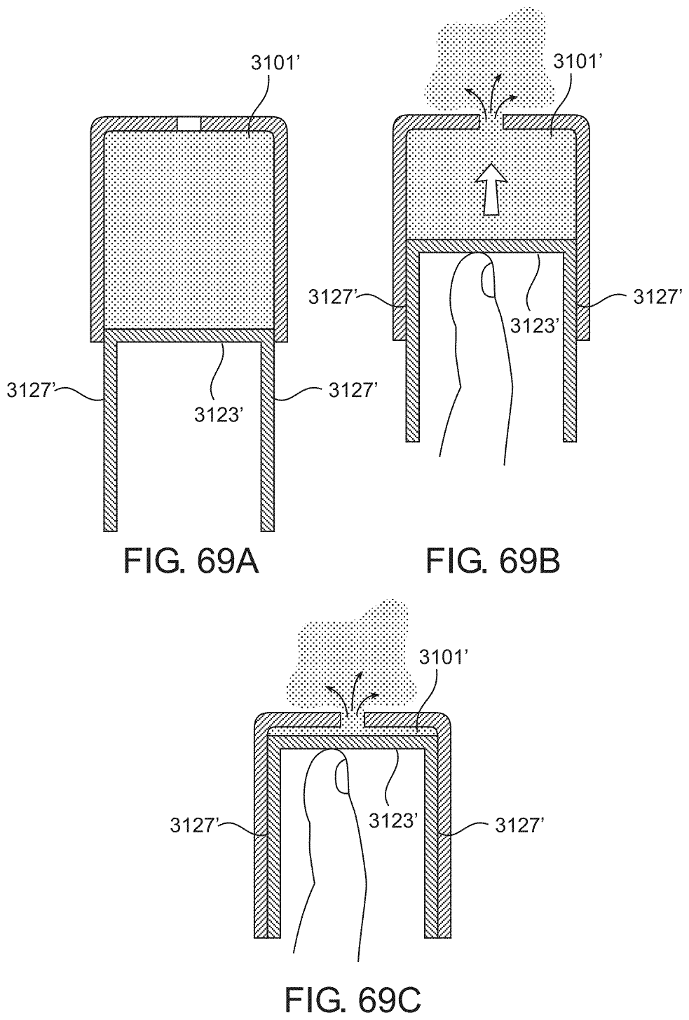

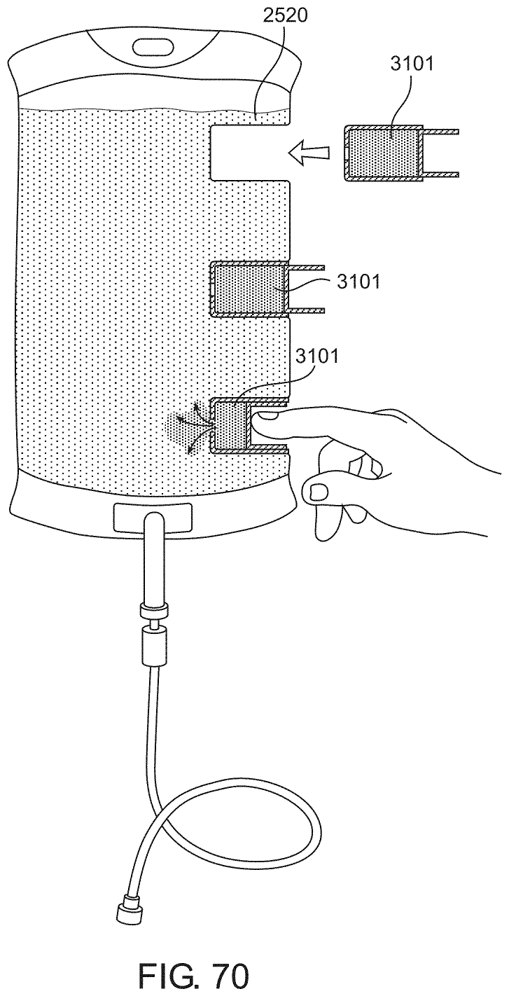

Yet a further form of cartridges is shown in 68-70. Cartridge 3101 includes a fluid transfer mechanism with a moveable plunger 3123 supported by side walls 3127. Side walls 3127 may be referred to as protective side walls as they protect a user's finger from contacting (being exposed) to any substance that may be housed/disposed inside cartridge 3101. Side walls (protective side walls) 3127 may be circumferential side walls. Side walls 3127 are of particular importance if the substance housed/disposed inside cartridge 3101 is a toxic substance such as a chemotherapeutic substance. As a user's finger pushes the moveable plunger 3123 into the cartridge and as a user's finger pushes the toxic substance disposed inside the cartridge out of the cartridge 3101 remnants of the toxic substance may remain disposed on inner side walls of the cartridge. Therefore, as a user's finger pushes the moveable plunger 3123 into cartridge 3101 thus ejecting toxic substance disposed inside cartridge 3101 out of cartridge 3101 exterior portion of side walls 3127 cover the inner side walls of the cartridge that have been exposed to the toxic substance. Therefore, remnants of the toxic substance are now entrapped between exterior portion of side walls 3127 and interior walls of the cartridge thus protecting a user from being exposed to the toxic substance. When pushed toward the opening of the cartridge 3103, the fluid is pushed outside and may fill a vessel. Side walls 3127′ may vary in length to be as long as cartridge side walls (as shown in FIGS. 69A-69C) such that when loaded into a vessel the cartridge protrudes from the walls of a vessel). In one or more embodiments, side walls 3127′ (FIGS. 69A-69C) may be longer (not shown) than side walls of the cartridge. A vessel 2520 having multiple cavities may be loaded with multiple cartridges 3101 having protective side walls (FIG. 70).

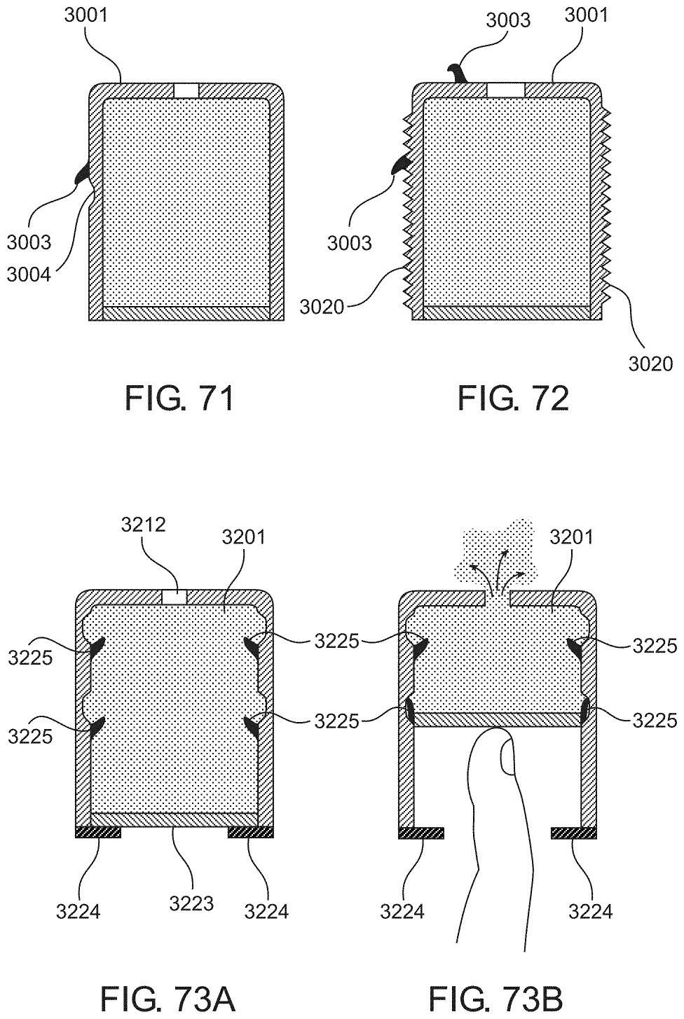

Various forms of locking mechanisms for allowing secure connection between the cartridge to a vessel are shown in FIGS. 71 and 72. The locking mechanism may include a locking member in the form of one or more knobs 3003 which may be locked into a complementary dent (not shown). Alternatively, or additionally, the locking mechanism may include external threads 3020. The knob 3003, dents 3004 and thread 3020 may all be disposed on various peripheral positions in/on cartridge 3001.

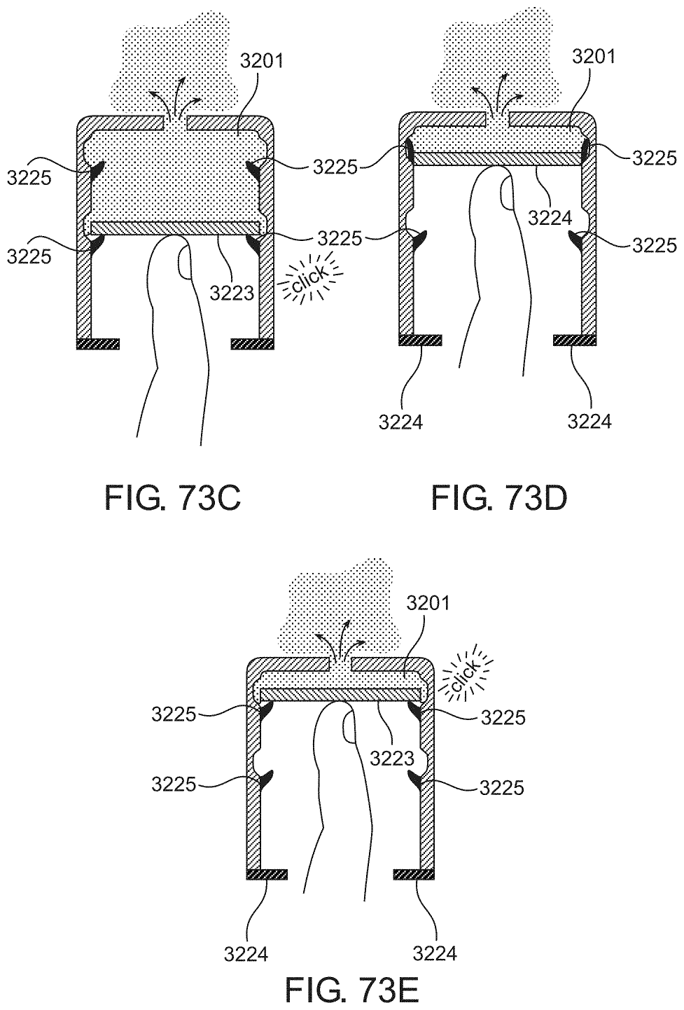

The herein disclosed vessel may optionally further include a safety mechanism for preventing retraction of fluid transfer mechanism. The following FIGS. 73A-77C illustrate possible safety mechanisms for ensuring a one-way movement of the fluid transfer mechanism. FIGS. 73A-73E illustrate cartridge 3201 which includes stoppers 3225 at opposing internal side walls thereof. Stoppers 3225 are retractable, optionally via a hinge (not shown) and lie flat against the side walls when moveable plunger 3223 is pushed towards opening 3212 and slides on top of the stoppers 3225. Optionally, two blockers 3224 may be disposed underneath plunger 3223 to block any retraction of the plunger 3223 out of the cartridge 3201. The stoppers 3225 may be made of a rigid or a semi-rigid material such as a plastic, an elastomeric substance, a metal alloy, a glass material, and combinations thereof. The blockers 3224 may be made of a rigid material such as a plastic, a metal alloy, and combinations thereof.

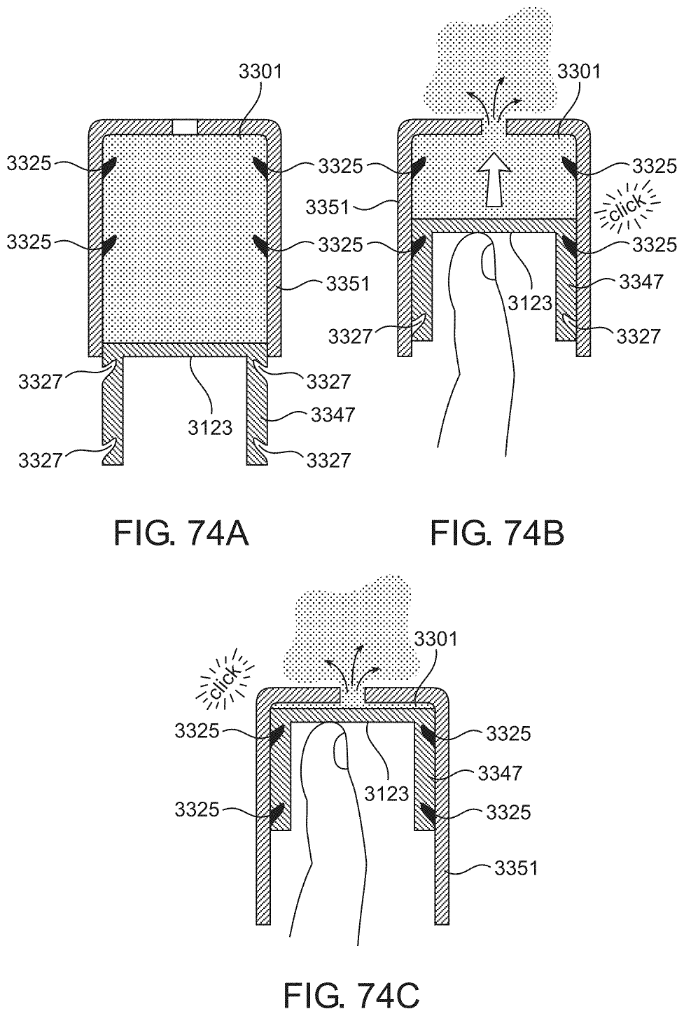

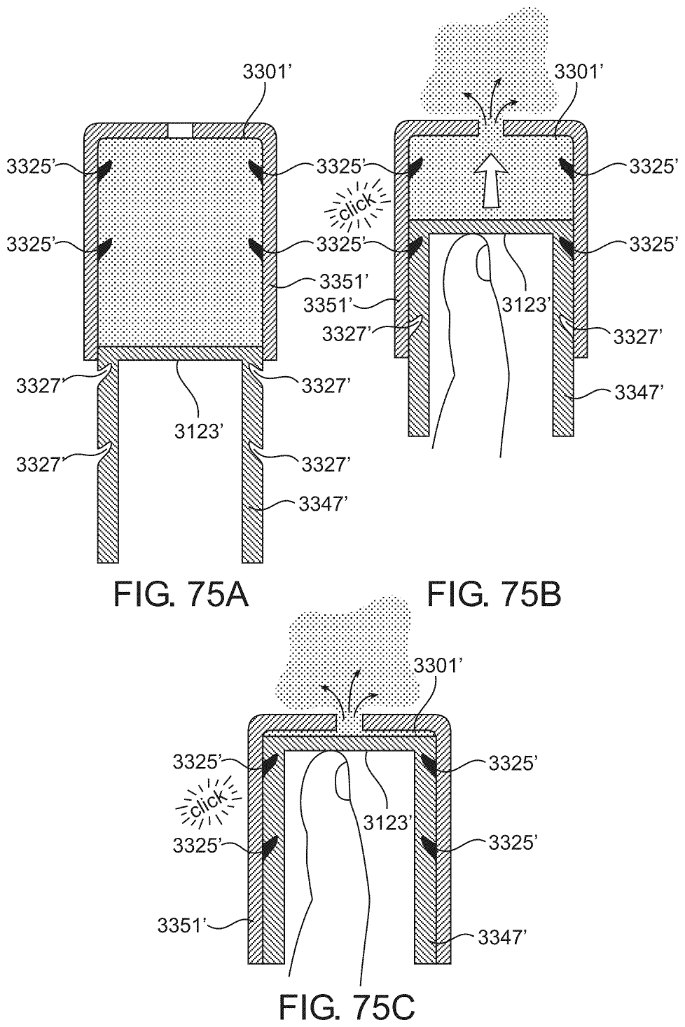

In FIGS. 74A-74C the plunger includes plunger dents 3327 configured to be coupled and lock stoppers 3325 thus ensuring a one-way movement of plunger 3123 towards the interior of cartridge 3301. Lock stoppers 3325 are configured to prevent plunger 3123 from retracting/detaching from cartridge 3301. Optionally plunger side walls 3347 may be shorter in length than the side walls 3351 of cartridge 3301, such that following the fluid transfer, walls 3347 are concealed by walls 3351. Alternatively, plunger side walls 3347′ may have a length that is similar (about the same) to the length of the side walls 3351′ of cartridge 3301′, such that following the fluid transfer the walls 3347′ and 3351′ are aligned (FIGS. 75A-75C). In one or more embodiments, plunger side walls 3347 may be a longer length (may be of a greater length) than side walls 3351 thus ensuring full entrapment of a toxic substance between exterior plunger side walls 3347 and interior of side walls 3351.

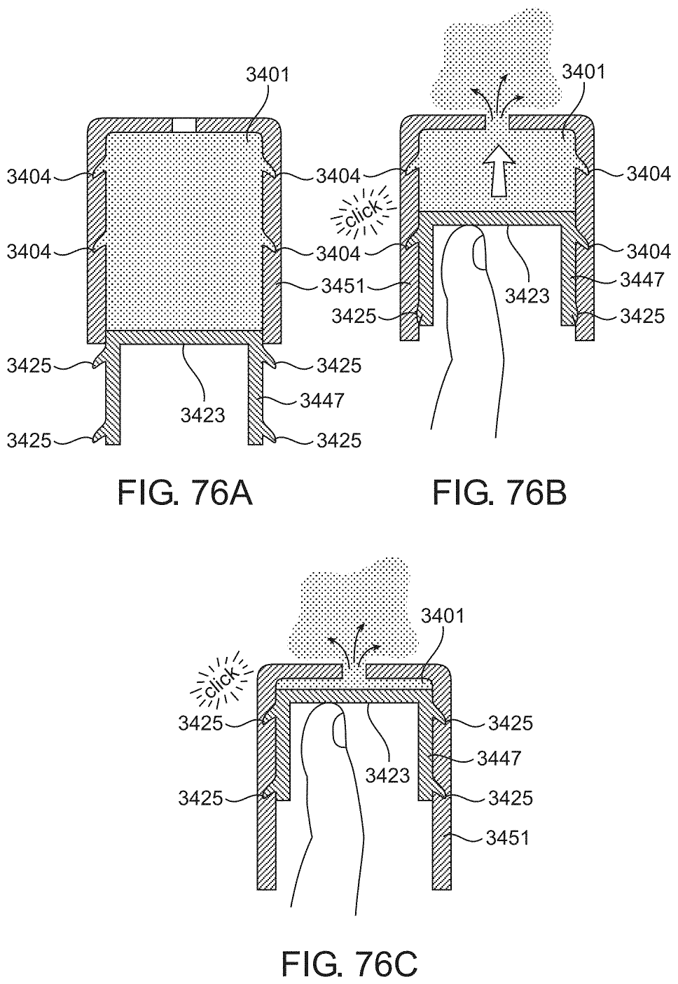

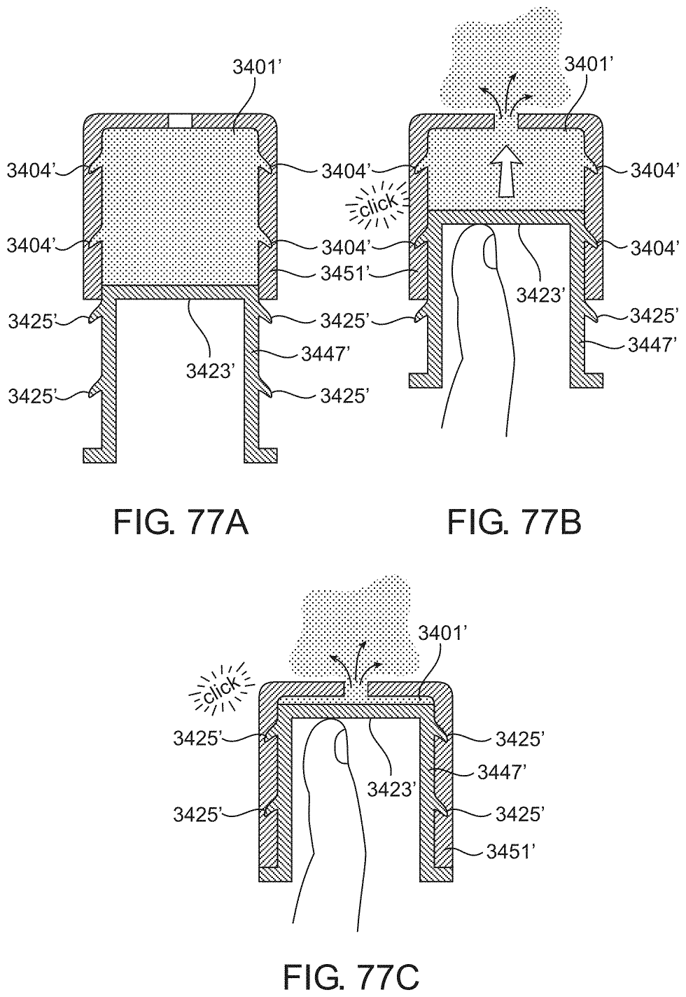

FIGS. 76A-77C illustrate yet another option wherein stoppers 3425 are disposed on the external surfaces of plunger walls 3447 and dents 3404 disposed in the internal walls of the side walls 3451 of the cartridge 3401. Optionally plunger side walls 3447 may be shorter in length than the side walls 3451 of cartridge 3401, such that following the fluid transfer walls 3447 are concealed by walls 3451 (FIGS. 76A-76C). Alternatively, plunger side walls 3447′ may have a length that is similar to the length of the side walls 3451′ of cartridge 3401′, such that following the fluid transfer the walls 3447 and 3451 are aligned (FIGS. 77A-77C). Walls 3447′ have edges that curve outwardly so as to cover exterior bottom of wall 3451′ when plunger 3423′ is pushed all the way into cartridge 3401′ (FIG. 77C). In one or more embodiments wall 3447′ may be longer than wall 3451′.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for the engagement of vessels, the system comprising:
a first vessel;
a second vessel configured to be coupled to the first vessel; and
a locking mechanism configured to permanently connect the vessels to each other, the locking mechanism comprising a locking member and a securing member, each disposed on a separate vessel, wherein the locking member configured to be coupled to the securing member and the securing member configured to hold the locking member.

2. The system of claim 1, wherein the locking member is selected from a hook, a tooth, a pin, a retractable pin, a ratchet tooth, a thread, a ball, a bangle, a locking key, a knob.

3. The system of claim 1, wherein the securing member is selected from a dent, a hole, a channel, and a slot.

4. The system of claim 1, wherein the locking member is moveable between a retracted position wherein the locking member abuts a wall of the vessel and an extended position, wherein the locking member protrudes from a wall of the vessel.

5. The system of claim 1, wherein at least one of the locking member and securing member disposed in an external position of the vessel.

6. The system of claim 1, wherein at least one of the locking member and securing member disposed in an internal position of the vessel.

7. The system of claim 1, wherein the locking member is pivotally connected to the vessel and moveable about said pivot.

8. The system of claim 1, wherein the locking member is spring loaded or attached to a spring, allowing planar movement of the locking member.

9. The system of claim 1, further comprising an engagement mechanism to connect the first vessel to the second vessel.

10. The system of claim 9, wherein the engagement mechanism is selected from a thread, a luer, a ratchet teeth mechanism, a retention member, an adhesive mechanism, a clamping member and combinations thereof.

11. The system of claim 9, wherein the engagement mechanism disposed in an external position of the vessel.

12. The system of claim 9, wherein the engagement mechanism disposed in an internal position of the vessel.

13. The system of claim 1, wherein at least one of the first vessel and second vessel is selected from the group consisting of a bottle, a syringe, an infusion line, a connector, a filter, a manifold, a bag port, a bottle port, a vial port, a cartridge and a combination thereof

14. The system of claim 1, wherein the first and/or second vessel includes walls enclosing a cavity, the cavity configured to receive or engage with a vessel, or a portion thereof.

15. The system of claim 14, wherein the locking member and/or the securing member disposed on an internal and/or external position of the cavity.

16. The system of claim 9, wherein the first and/or second vessel includes a port having peripheral walls enclosing a cavity, the cavity configured to receive or engage with a vessel, or a portion thereof, wherein the engagement mechanism disposed on an internal and/or external position of the port.

17. The system of claim 1, wherein the first or second vessel is configured to be coupled to a plurality of vessels.

18. The system of claim 1, wherein the first vessel is configured to accommodate a fluid substance and the second vessel configured to receive a fluid substance from the first vessel.

19. The system of claim 18, wherein the first vessel is a cartridge having plunger side walls.

20. The system of claim 19, wherein the first and/or second vessel includes a port having peripheral walls enclosing a cavity, the cavity is sized and shaped to accommodate the cartridge.

21. The system of claim 20, wherein when engaged, an external surface of a wall of the cartridge protrudes from an external surface of a wall of the second vessel.

22. The system of claim 20, wherein when engaged, an external surface of a wall of the cartridge is aligned with an external surface of a wall of the second vessel.

23. The system of claim 20, wherein when engaged, an external surface of a wall of the cartridge is entirely concealed within the walls of the second vessel.

24. The system of claim 19, wherein a plurality of cartridges are configured to be coupled to the second vessel.

25. The system of claim 18, wherein the first vessel includes a fluid transfer mechanism for transferring a fluid substance from the first vessel to the second vessel.

26. The system of claim 25, wherein the fluid transfer mechanism includes a moveable plunger which when moved towards the first vessel's opening, allows the fluid transfer.

27. The system of claim 25, further comprising a safety mechanism for preventing retraction of the fluid transfer mechanism.

28. The system of claim 1, wherein a hermetically sealed connection is formed between said first vessel and said second vessel following the vessels' engagement.
